(19) United States
(12) Patent Application Publication
(10) Pub. No.: US 2023/0407272 A1
(43) Pub. Date: Dec. 21, 2023

(54) GENETICALLY ENGINEERED YEAST CELLS AND METHODS OF USE THEREOF

(71) Applicant: Berkeley Fermentation Science Inc., Oakland, CA (US)

(72) Inventors: Rachel Li, Berkeley, CA (US); Jeremy Roop, Berkeley, CA (US); Nicholas Harris, Glenwood Springs, CO (US); Charles Denby, Berkeley, CA (US)

(73) Assignee: Berkeley Fermentation Science Inc., Oakland, CA (US)

(21) Appl. No.: 18/036,472

(22) PCT Filed: Nov. 12, 2021
(86) PCT No.: PCT/US2021/059201

§ 371 Date: May 11, 2023

(51) Int. Cl. C12N 9/10; C12C 12/00; C12G 1/022

(52) U.S. Cl. CPC C12N 9/1029; C12Y 203/01084; C12Y 203/01086; C12C 12/004; C12C 12/006; C12G 1/0203; C12G 2200/05; C12G 2200/11; C12C 2200/05

(57) ABSTRACT

Provided herein are genetically modified yeast cells that recombinantly express a gene encoding an alcohol-O-acyltransferase (AAT) enzyme and a gene encoding a fatty acid synthase a subunit (FAS2) enzyme. Also provided are methods of producing fermented beverages and compositions comprising ethanol using the genetically modified yeast cells described herein.

### RELATED APPLICATION

This application claims the benefit under 35 U.S.C. § 119(e) of U.S. Provisional Application No. 63/113,747, filed Nov. 13, 2020, the entire disclosure of which is hereby incorporated by reference in its entirety.

### GOVERNMENT SUPPORT

This invention was made with government support under Award Number 1831242 awarded by the National Science Foundation. The government has certain rights in the invention.

### BACKGROUND

Fruity, and tropical fruit flavors are highly desirable in the fermented beverage market. Wines that impart fruity flavors, like Chardonnays and Sauvignon Blancs, make up the majority of wine sales in the US (Statista (2019), Wine Consumption by Category, United States), while the popularity of beers made with fruity flavoring hops has skyrocketed in the last decade (Craft Beer Club (2018), Your Guide to the Most Popular Beer Hops in the USA; Watson (2018), Beer Style Trends). The fruity flavors present in both beer and wine result from the presence of volatile flavor-active molecules that, when present in concentrations above the human detection threshold, impart fruity aromas and tastes. One flavor-molecule that imparts fruity tasting notes is the ester ethyl-hexanoate. Ethyl-hexanoate is the principal contributor to the flavor of pineapples but also is an integral component of other fruity flavors like mango, guava, and apple (Reddy et al. Indian J. Microbiol. (2010). 50:183-191; Zheng et al. Int. J. Mol. Sci. (2012). 13:7383-7392; Kaewtathip et al. Int. J. Food Sci. & Tech. (2012). 47:985-990; Espino-Diaz et al. Food Technol. Biotechnol. (2016). 54:375).

### SUMMARY

The present disclosure provides, in some aspects, genetically modified yeast cells comprising a gene encoding an enzyme with alcohol-O-acyltransferase (AAT) activity and a gene encoding an enzyme with fatty acid synthase (FAS2) activity. Enzymes with AAT activity catalyze the reaction of ethanol with hexanoic acid or hexanoyl-CoA to form the fatty acid ester ethyl-hexanoate, which imparts a fruity, pineapple flavor to fermented beverages such as beer and wine. Modified cells with AAT activity may thus produce ethyl-hexanoate during fermentation, thereby imparting such flavors to the resulting fermented beverages, though may also produce hexanoic acid, a pungent fatty acid that can impart undesired, cheesy, rancid, and goaty flavors when present at concentrations above a flavor detection threshold. Enzymes with FAS2 activity function to extend fatty acid chains. Modified cells with altered FAS2 activity may thus produce short fatty acid chains (e.g., in the form of hexanoyl-CoA), which is a precursor for producing ethyl hexanoate. The modified cells described herein further aim to minimize hexanoic acid production during fermentation, and thereby avoid imparting unpleasant flavors to the resulting fermented beverages. Modified cells of the present disclosure may also comprise a third gene encoding an enzyme with hexanoyl-CoA synthetase (HCS) activity. Enzymes with HCS activity catalyze the formation of hexanoyl-CoA from the substrates hexanoic acid and free coenzyme A (CoA). By converting hexanoic acid to a precursor of ethyl-hexanoate synthesis, modified cells with HCS activity may thus produce both more ethyl-hexanoate and less hexanoic acid during fermentation, imparting more desired flavors and fewer undesired ones to the resulting fermented beverage. The enzymes may be further modified to increase their production of ethyl-hexanoate or reduce production of hexanoic acid, and the genes encoding the enzymes may be operably linked to promoters to further increase ethyl-hexanoate or decrease hexanoic acid production.

The present disclosure provides, in some aspects, genetically modified yeast cells (modified cells), comprising a first gene encoding an enzyme with alcohol-O-acyltransferase (AAT) activity operably linked to a first promoter, and a second gene encoding an enzyme with fatty acid synthase (FAS2) activity operably linked to a second promoter. In some embodiments, the enzyme having AAT activity is derived from Marinobacter hydrocarbonoclasticus, Fragraia x ananassa, Saccharomyces cerevisiae, Neurospora sitophila, Actinidia deliciosa, Actinidia chinensis, Marinobacter aquaeolei, Saccharornycopsis fibuligera, Malus x domestica, Solanum pennellii, or Solanum lycopersicum. In some embodiments, the enzyme having AAT activity comprises a sequence having at least 90% sequence identity to the amino acid sequence set forth in SEQ ID NO: 2-4 or 12-22. In some embodiments, the enzyme having AAT activity does not comprise the sequence of SEQ ID NO: 1. In some embodiments, the enzyme having AAT activity comprises the sequence of SEQ ID NO: 20.

In some embodiments, the first enzyme having AAT activity comprises at least one substitution mutation at a position corresponding to position A144 and/or A360 of SEQ ID NO: 1. In some embodiments, the substitution mutation at the position corresponding to position 144 of SEQ ID NO: 1 is a phenylalanine. In some embodiments, the substitution mutation at the position corresponding to position 360 of SEQ ID NO: 1 is an isoleucine.

In some embodiments, the enzyme having AAT activity comprises at least one substitution mutation at a position corresponding to position A169 and/or A170 of SEQ ID NO: 19. In some embodiments, the substitution mutation at the position corresponding to position 169 of SEQ ID NO: 19 is a glycine. In some embodiments, the substitution mutation at the position corresponding to position 170 of SEQ ID NO: 19 is a phenylalanine. In some embodiments, the first enzyme having AAT activity comprises a substitution mutation at a position corresponding to position G150 of a wild-type MhWES2 amino acid sequence. In some embodiments, the substitution mutation at the position corresponding to position G150 of a wild-type MhWES2 amino acid sequence is a phenylalanine.

In some embodiments, the enzyme having FAS2 activity is derived from Saccharomyces cerevisiae. In some embodiments, the enzyme having FAS2 activity comprises a sequence having at least 90% sequence identity to the sequence of SEQ ID NO: 6. In some embodiments, the enzyme having FAS2 activity does not comprise the sequence of SEQ ID NO: 5. In some embodiments, the enzyme having FAS2 activity comprises a substitution mutation at a position corresponding to position 1250 of SEQ ID NO: 5. In some embodiments, the substitution mutation at the position corresponding to position 1250 of SEQ ID NO: 5 is a serine.

In some embodiments, the modified cell further comprises a third heterologous gene operably linked to a third promoter, wherein the third heterologous gene encodes an enzyme having hexanoyl-CoA synthetase (HCS) activity. In some embodiments, the enzyme having HCS activity is derived from Cannabis sativa. In some embodiments, the enzyme having HCS activity comprises a sequence having at least 90% sequence identity to the sequence of SEQ ID NO: 7.

In some embodiments, the first promoter and/or the second promoter is selected from the group consisting of pHEM13, pSPG1, pPRB1, pQCR10, pPGK1, pOLE1, pERG25, and pHHF2. In some embodiments, the first promoter is pHEM13, and the second promoter is pSPG1. In other embodiments, the first promoter is pHEM13, and the second promoter is pPRB1. In yet other embodiments, the first promoter is pQCR10, and the second promoter is pPRB1. In yet other embodiments, the first promoter is pPGK, and the second promoter is pPRB1.

In some embodiments, the third promoter is selected from the group consisting of pHEM13, pSPG1, pPRB1, pQCR10, pPGK1, pOLEl, pERG25, and pHHF2. In some embodiments, the first promoter is pHEM13, the second promoter is pPRB1, and the third promoter is pHEM13. In other embodiments, the first promoter is pQCR10, the second promoter is pPRB1, and the third promoter is pHEM13. In other embodiments, the first promoter is pPGK1, the second promoter is pPRB1, and the third promoter is pERG25.

In some embodiments, the cell has been genetically modified to reduce expression of one or more endogenous AAT enzymes. In some embodiments, the modified cell does not express endogenous EEB1, EHT1, and/or MGL2.

In some embodiments, the yeast cell is of the genus Saccharomyces. In some embodiments, the yeast cell is of the species Saccharomyces cerevisiae (S. cerevisiae). In some embodiments, the yeast cell is S. cerevisiae California Ale Yeast strain WLP001, EC-1118, Elegance, Red Star Côte des Blancs, or Epernay II. In some embodiments, the yeast cell is of the species Saccharomyces pastorianus (S. pastorianus).

In some embodiments, the growth rate of the modified cell is not substantially impaired relative to a wild-type yeast cell that does not comprise the first heterologous gene and second exogenous gene. In some embodiments, within one month of the start of fermentation, the modified cell ferments a comparable amount of fermentable sugar to the amount fermented by wild-type yeast cell that does not comprise the first heterologous gene and second exogenous gene. In some embodiments, within one month of the start of fermentation, the modified cell reduces the amount of fermentable sugars in a medium by at least 95%. In some embodiments, the cell comprises an endogenous gene encoding an enzyme having FAS2 activity.

Some aspects of the present disclosure provide methods of making a fermented product, comprising contacting a modified cell with a medium comprising at least one fermentable sugar, wherein the contacting is performed during at least a first fermentation process, to produce a fermented product. In some embodiments, at least one fermentable sugar is provided in at least one sugar source. In some embodiments, the fermentable sugar is glucose, fructose, sucrose, maltose, and/or maltotriose.

In some embodiments, the fermented product comprises an increased level of at least one desired product as compared to a fermented product produced by a counterpart cell that does not express the first, second, and/or third heterologous genes, or a counterpart cell that expresses a wild-type enzyme having AAT activity. In some embodiments, the desired product is ethyl-hexanoate.

In some embodiments, the fermented product comprises a reduced level of at least one undesired product as compared to a fermented product produced by a counterpart cell that does not express the first heterologous gene, second exogenous gene, and/or third heterologous genes, or a counterpart cell that expresses a wild-type enzyme having AAT activity. In some embodiments, at least one undesired product is hexanoic acid.

In some embodiments, the fermented product is a fermented beverage. In some embodiments, the fermented beverage is beer, wine, sparkling wine (champagne), wine cooler, wine spritzer, hard seltzer, sake, mead, kombucha, or cider.

In some embodiments, the sugar source comprises wort, must, fruit juice, honey, rice starch, or a combination thereof. In some embodiments, the fruit juice is a juice obtained from at least one fruit selected from the group consisting of grapes, apples, blueberries, blackberries, raspberries, currants, strawberries, cherries, pears, peaches, nectarines, oranges, pineapples, mangoes, and passionfruit.

In some embodiments, the sugar source is wort and the method further comprises producing the medium, wherein producing the medium comprises: (a) contacting a plurality of grains with water; and (b) boiling or steeping the water and grains to produce wort. In some embodiments, the method further comprises adding at least one hop variety to the wort to produce a hopped wort. In some embodiments, the method further comprises adding at least one hop variety to the medium.

In some embodiments, the sugar source is must and the method further comprises producing the medium, wherein producing the medium comprises crushing a plurality of fruits to produce the must. In some embodiments, the method further comprises removing solid fruit material from the must to produce a fruit juice.

In some embodiments, the method comprises at least one additional fermentation process. In some embodiments, the method comprises carbonating the fermented product.

The present disclosure provides, in some aspects, a fermented product produced, obtained, or obtainable by one of the methods described herein. In some embodiments, the fermented product comprises at least 200 μg/L ethyl-hexanoate. In some embodiments, the fermented product comprises less than 10 mg/L hexanoic acid.

Some aspects of the present disclosure provide methods of producing a composition comprising ethanol, the method comprising contacting a modified cell with a medium comprising at least one fermentable sugar, wherein the contacting is performed during at least a first fermentation process, to produce a composition comprising ethanol.

In some embodiments, at least one fermentable sugar is provided in at least one sugar source. In some embodiments, the fermentable sugar is glucose, fructose, sucrose, maltose, and/or maltotriose.

In some embodiments, the composition comprising ethanol comprises an increased level of at least one desired product as compared to a composition comprising ethanol produced by a counterpart cell that does not express the first, second, and/or third heterologous genes, or a counterpart cell that expresses a wild-type enzyme having AAT activity. In some embodiments, the desired product is ethyl-hexanoate.

In some embodiments, the composition comprising ethanol comprises a reduced level of at least one undesired product as compared to a composition comprising ethanol produced by a counterpart cell that does not express the first heterologous gene, second exogenous gene, and/or third heterologous genes, or a counterpart cell that expresses a wild-type enzyme having AAT activity. In some embodiments, at least one undesired product is hexanoic acid.

In some embodiments, the composition comprising ethanol is a fermented beverage. In some embodiments, the fermented beverage is beer, wine, sparkling wine (champagne), wine cooler, wine spritzer, hard seltzer, sake, mead, kombucha, or cider.

In some embodiments, the sugar source comprises wort, must, fruit juice, honey, rice starch, or a combination thereof. In some embodiments, the fruit juice is a juice obtained from at least one fruit selected from the group consisting of grapes, apples, blueberries, blackberries, raspberries, currants, strawberries, cherries, pears, peaches, nectarines, oranges, pineapples, mangoes, and passionfruit.

In some embodiments, the sugar source is wort and the method further comprises producing the medium, wherein producing the medium comprises: (a) contacting a plurality of grains with water; and (b) boiling or steeping the water and grains to produce wort. In some embodiments, the method further comprises adding at least one hop variety to the wort to produce a hopped wort. In some embodiments, the method further comprises adding at least one hop variety to the medium.

In some embodiments, the sugar source is must and the method further comprises producing the medium, wherein producing the medium comprises crushing a plurality of fruits to produce the must. In some embodiments, the method further comprises removing solid fruit material from the must to produce a fruit juice.

In some embodiments, the method comprises at least one additional fermentation process. In some embodiments, the method comprises carbonating the composition comprising ethanol.

The present disclosure provides, in some aspects, a composition comprising ethanol produced, obtained, or obtainable by one of the methods described herein. In some embodiments, the composition comprising ethanol comprises at least 200 μg/L ethyl-hexanoate. In some embodiments, the composition comprising ethanol comprises less than 10 mg/L hexanoic acid.

### BRIEF DESCRIPTION OF THE DRAWINGS

Further aspects of the disclosure will be readily appreciated upon review of the Detailed Description of its various aspects and embodiments, described below, when taken in conjunction with the accompanying Drawings.

FIGS. 1A and 1B show ethyl hexanoate and hexanoic acid production by engineered beer brewing yeast strains in malt extract fermentations. FIG. 1A shows the fold change of ethyl hexanoate and hexanoic acid production by engineered brewing yeast strains as compared to the parental wild-type Saccharomyces cerevisiae CA01 strain. FIG. 1B shows concentrations of ethyl hexanoate (mg/L) and hexanoic acid (mg/L) produced by Saccharomyces cerevisiae strain y1210 or the wild-type Saccharomyces cerevisiae CA01 strain. Each bar in reports the average of two biological replicates. Error bars indicate standard deviation. Strains correspond to wild-type Saccharomyces cerevisiae CA01 (CA01); CA01 expressing FAS2_G1250S and MpAAT1_A169G/A170F (y1059); CA01 expressing FAS2_G1250S and MpAAT1_A169G/A170F and comprising a deletion EHT1 (y1227); CA01 expressing FAS2_G1250S and MpAAT1_A169G/A170F and comprising deletions of EHT1 and EEB1 (y1076); CA01 expressing FAS2_G1250S and MpAAT1_A169G/A170F and comprising deletions of EHT1, EEB1, and MGL2 (y1170); CA01 expressing FAS2_G1250S, MpAAT1_A169G/A170F, and HCS and comprising deletions of EHT1, EEB1, and MGL2 (y1210); and CA01 expressing FAS2 and MpAAT1_A169G/A170F and comprising deletions of EHT1 and EEB1 (y1232).

FIGS. 2A and 2B show ethyl hexanoate and hexanoic acid production by engineered wine yeast strains in grape juice fermentations. FIG. 2A shows concentrations of ethyl hexanoate (mg/L) and hexanoic acid (mg/L) produced by engineered wine yeast strains and the wild-type parental Saccharomyces cerevisiae EC1118 strain. FIG. 2B shows the ratio of ethyl hexanoate to hexanoic acid produced by each of the indicated strains. Ethyl hexanoate and hexanoic acid concentration values are derived from FIG. 2A. Each bar reports the average of two biological replicates. Error bars indicate standard deviation. Strains correspond to wild-type S. cerevisiae EC1118 (EC1118), S. cerevisiae Elegance expressing FAS2 G1250S and MaWES1-A144F/A360I (SEQ ID NO: 4; y786); S. cerevisiae Elegance expressing FAS2_G1250S and MaWES1 and comprising deletions of EHT1 and EEB1 (y1080); S. cerevisiae EC1118 expressing FAS2_G1250S and MaWES1 (y796); S. cerevisiae EC1118 expressing FAS2_G1250S and MaWES1 and comprising deletions of EHT1 and EEB1 (y1115); S. cerevisiae EC1118 expressing FAS2 G1250S and MpAAT1_A169G/A170F (y1134); and S. cerevisiae EC1118 expressing FAS2 G1250S and MpAAT1_A169G/A170F and comprising deletions of EHT1 and EEB1 (y1138).

### DETAILED DESCRIPTION

Fruity and tropical fruit flavors are highly desirable to consumers in the fermented beverage market. Pineapple, guava, and berry flavors are especially popular, as evidenced by the robust sales of Chardonnay and Sauvignon Blanc wines, and beers produced with tropical-aroma flavoring hops. The presence of these flavors in both fruits and fermented beverages is due to various flavor-active molecules that collectively impart distinctive tastes and aromas when consumed. One such molecule, ethyl-hexanoate, contributes to many fruity and tropical fruit flavors. In isolation, ethyl-hexanoate is perceived as pineapple, but it also contributes to the flavor of mango, apple, guava, and many other fruits. The genetically modified yeast cells and methods described herein aim to increase concentrations of ethyl-hexanoate produced during fermentation, such as for production of beer or wine.

Several groups have attempted to engineer yeast strains for increased production of ethyl-hexanoate during the fermentation process. However, these efforts have not led to the development of commercially viable yeast with enhanced ethyl-hexanoate production due to challenges in balancing strain phenotypes of increasing production of ethyl-hexanoate, unaltered growth rate, and minimal production of the off-flavor molecule, hexanoic acid. In contrast, the genetically modified cells described herein are capable of producing increased levels of ethyl-hexanoate, reduced levels of off-flavors (e.g., hexanoic acid), and have substantially unaltered growth characteristics.

Concentrations of ethyl-hexanoate vary greatly between different beer and wine styles, from less than 100 μg/L to over 1500 μg/L (see, e.g., Avram et al. Anal. Lett. (2015). 48:1099-1116; Niu et al. J. Chromatogr. B. (2011). 879:2287-2293; Holt et al. FEMS Microbiol Rev. (2019). 43:193-222). This variation in ethyl-hexanoate concentration is due in part to differences in the specific grape, barley, or hop varietals that are used as starting materials for these fermentations, but it is also influenced by the yeast strain used in the fermentation process. Some yeast strains may produce fermented beverages with fruity flavors, but the concentration of ethyl-hexanoate produced is often barely above the threshold of detection for humans. Consequently, the fruity flavors associated with ethyl-hexanoate are often subtle, or barely noticeable, especially after the addition of other components to the beverage, such as potent flavoring hops.

Provided herein are genetically modified yeast cells that have been engineered to express an enzyme having alcohol-O-acyltransferase (AAT) activity and an enzyme having fatty acid synthase (FAS2) activity. In some embodiments, the enzyme having AAT activity has been modified to increase production of ethyl-hexanoate and/or reduce production of undesired hexanoic acid. Also provided herein are methods of producing a fermented beverage involving contacting the genetically modified yeast cells with a medium comprising a sugar source comprising at least one fermentable sugar during a fermentation process. Also provided herein are methods of producing ethanol, including composition comprising ethanol, involving contacting the genetically modified yeast cells with a medium comprising a sugar source comprising at least one fermentable sugar during a fermentation process.

### Alcohol-O-acyltransferase (AAT) Enzymes

The genetically modified cells described herein contain a gene encoding an enzyme with alcohol-O-acyltransferase (AAT) activity. In some embodiments, the gene is a heterologous gene. The term “heterologous gene,” as used herein, refers to a sequence of nucleic acid (e.g., DNA) that contains the genetic instruction, which is introduced into and expressed by a host organism (e.g., a genetically modified cell) which does not naturally encode the introduced gene. The heterologous gene may encode an enzyme that is not typically expressed by the cell or a variant of an enzyme that the cell does not typically express (e.g., a mutated enzyme).

Alcohol-O-acyltransferases, which may also be referred to as acetyl-CoA:acetyltransferases or alcohol acetyltransferases, are bisubstrate enzymes that catalyze the transfer of acyl chains from an acyl-coenzyme A (CoA) donor to an acceptor alcohol, resulting in the production of an acyl ester. The acyl esters present in a fermented beverage influence its flavor. The ester ethyl-hexanoate, which is formed by the condensation of ethanol and either hexanoic acid or hexanoyl-CoA, imparts a pineapple flavor to fermented beverages such as beer and wine.

In some embodiments, the heterologous gene encoding an enzyme with alcohol-O-acyltransferase activity is a wild-type alcohol-O-acyltransferase gene (e.g., a gene isolated from an organism). In some embodiments, the heterologous gene encoding an enzyme with alcohol-O-acyltransferases activity is a mutant alcohol-O-acyltransferases gene and contains one or more mutations (e.g., substitutions, deletions, insertions) in the nucleic acid sequence of the alcohol-O-acyltransferase gene and/or in amino acid sequence of the enzyme having alcohol-O-acyltransferase activity. As will be understood by one of ordinary skill in the art, mutations in a nucleic acid sequence may change the amino acid sequence of the translated polypeptide (e.g., substitution mutation) or may not change the amino acid sequence of the translated polypeptide (e.g., silent mutations) relative to a wild-type enzyme or a reference enzyme.

In some embodiments, the heterologous gene encoding an enzyme with alcohol-O-acyltransferase activity is a truncation, which is deficient in one or more amino acids, preferably at the N-terminus or the C-terminus of the enzyme, relative to a wild-type enzyme or a reference enzyme.

In some embodiments, the alcohol-O-acyltransferase is obtained from a bacterium or a fungus, including a yeast. In some embodiments, the alcohol-O-acyltransferase is obtained from Marinobacter hydrocarbonoclasticus, Saccharomyces cerevisiae, Neurospora sitophila, Fragaria x ananassa, Actinidia deliciosa, Actinidia chinensis, Marinobacter aquaeolei, Saccharornycopsis fibuligera, Malus x domestica, or Solanum pennellii.

An exemplary alcohol-O-acyltransferase is MaWES from Marinobacter aquaeolei, which is provided by the Accession No. WP_011783747.1 and amino acid sequence set forth as SEQ ID NO: 1.

Amino acid sequence of wildtype MaWES from Marinobacter aquaeolei

(SEQ ID NO: 1)
MTPLNPTDQLFLWLEKRQQPMHVGGLQLFSFPEGAPDDYVAQLADQLRQKTEVTAPFNQRLSYRLGQPVWVEDEH
LDLEHHFRFEALPTPGRIRELLSFVSAEHSHLMDRERPMWEVHLIEGLKDRQFALYTKVHHSLVDGVSAMRMATR
MLSENPDEHGMPPIWDLPCLSRDRGESDGHSLWRSVTHLLGLSGRQLGTIPTVAKELLKTINQARKDPAYDSIFH
APRCMLNQKITGSRRFAAQSWCLKRIRAVCEAYGTTVNDVVTAMCAAALRTYLMNQDALPEKPLVAFVPVSLRRD
DSSGGNQVGVILASLHTDVQEAGERLLKIHHGMEEAKQRYRHMSPEEIVNYTALTLAPAAFHLLTGLAPKWQTEN
VVISNVPGPSRPLYWNGAKLEGMYPVSIDMDRLALNMTLTSYNDQVEFGLIGCRRTLPSLQRMLDYLEQGLAELE
LNAGL

In some embodiments, the alcohol-O-acyltransferase is a homolog of MaWES from Marinobacter aquaeolei (SEQ ID NO: 1). Homologs or related enzymes may be identified using methods known in the art, such as those described herein.

In some embodiments, the alcohol-O-acyltransferase is obtained from a plant, such as crop plant. In some embodiments, the alcohol-O-acyltransferase is from a strawberry plant. In some embodiments, the alcohol-O-acyltransferase gene is from a Fragraia species. In some embodiments, the alcohol-O-acyltransferase gene is from Fragraia x ananassa. The amino acid sequence of the wild-type MaWES homolog from F. x ananassa is given by Accession No. AAG13130.1 and has 17% sequence identity to MaWES from Marinobacter aquaeoleis (SEQ ID NO: 1). The catalytic histidine within the highly conserved HXXXD[A/G] motif is indicated in boldface in SEQ ID NO: 2 below. This motif is highly conserved across AAT enzymes in plants and bacterial species. The plant homologs also have a highly conserved [N/D]FGWG (SEQ ID NO: 23) motif indicated below with underlining.

Amino acid sequence of wildtype alcohol-O-acyltransferase from Fragraia x ananassa

(SEQ ID NO: 2)
MGEKIEVSINSKHTIKPSTSSTPLQPYKLILLDQLTPPAYVPIVFFYPITDHDFNLPQTAADLRQALSETLTLYY
PLSGRVKNNLYIDDFEEGVPYLEARVNCDMTDFLRLRKIECLNEFVPIKPFSMEAISDERYPLLGVQVNVEDSGI
AIGVSVSHKLIDGGTADCFLKSWGAVFRGCRENIIHPSLSEAALLFPPRDDLPEKYVDQMEALWFAGKKVATRRF
VFGVKAISSIQDEAKSESVPKPSRVHAVTGFLWKHLIAASRALISGTTSTRLSIAAQAVNLRTRMNMETVLDNAT
GNLFWWAQAILELSHTTPEISDLKLCDLVNLLNGSVKQCNGDYFETFKGKEGYGRMCEYLDFQRTMSSMEPAPDI
YLFSSWTNFFNPLDFGWGRISWIGVAGKIESASCKFIILVPTQCGSGIEAWVNLEEEKMAMLEQDPHFLALASPK
TLI

An exemplary alcohol-O-acyltransferase is SAAT from Fragaria x ananassa, as described, for example, in Beekwilder J, et al. Plant Physiol. (2004) 135(4):1865-78). In some embodiments, the amino acid sequence SAAT from Fragaria x ananassa is set forth as SEQ ID NO: 14.

(SEQ ID NO: 14)
MGEKIEVSINSKHTIKPSTSSTPLQPYKLTLLDQLTPPAYVPIVFFYPITDHDFNLPQTLADLRQALSETLTLYY
PLSGRVKNNLYIDDFEEGVPYLEARVNCDMTDFLRLRKIECLNEFVPIKPFSMEAISDERYPLLGVQVNVFDSGI
AIGVSVSHKLIDGGTADCFLKSWGAVERGCRENIIHPSLSEAALLFPPRDDLPEKYVDQMEALWFAGKKVATRRF
VFGVKAISSIQDEAKSESVPKPSRVHAVIGFLWKHLIAASRALTSGTTSTRLSIAAQAVNLRTRMNMETVLDNAT
GNLFWWAQAILELSHTTPEISDLKLCDLVNLLNGSVKQCNGDYFETFKGKEGYGRMCEYLDFQRTMSSMEPAPDI
YLFSSWINFFNPLDFGWGRISWIGVAGKIESASCKFIILVPTQCGSGIEAWVNLEEEKMAMLEQDPHFLALASPK
TLI

In some embodiments, the alcohol-O-acyltransferase is from a tomato plant. In some embodiments, the alcohol-O-acyltransferase gene is from a Solanum species. In some embodiments, the alcohol-O-acyltransferase gene is from Solanum lycopersicum. In some embodiments, the alcohol-O-acyltransferase is from Solanum pennellii. An exemplary alcohol-O-acyltransferase is SpAAT1 from Solanum pennellii, as described, for example, in Goulet C, et al. Molecular Plant (2015) 8: 1, 153-162. The amino acid sequence of the wild-type MaWES homolog from Solanum pennellii is given by Accession No. NP_001310384.1 and has 15% sequence identity to MaWES from Marinobacter aquaeolei (SEQ ID NO: 1). In some embodiments, the amino acid sequence of SpAAT1 from Solanum pennellii is set forth as SEQ ID NO: 3.

(SEQ ID NO: 3)
MANTLPISINYHKPKLVVPSSVTPHETKRLSEIDDQGFIRFQIPILMFYKYNSSMKGKDPARIIEDGLSKILVFY
HPLAGRLIEGPNKKLMVNCNGEGVLFIEGDANIELEKLGESIKPPCPYLDLLLHNVPGSDGIIGSPLLLIQVTRF
TCGGFAVGFRVSHTMMDGYGFKMFLNALSELIQGASTPSILPVWQRHLLSARSSPCITCSHHEFDEEIESKIAWE
SMEDKLIQESFFFGNEEMEVIKNQIPPNYGCTKFELLMAFLWKCRTIALDLHPEEIVRLTYVINIRGKKSLNIEL
PIGYYGNAFVTPVVVSKAGLLCSNPVTYAVELIKKVKDHINEEYIKSVIDLIVIKGRPELTKSWNFLVSDNRYIG
FDEFDFGWGNPIFGGISKATSFISFGVSVKNDKGEKGVLIAISLPPLAMKKLQDIYNMTFRVIIPRI

In some embodiments, the alcohol-O-acyltransferase is from Saccharomyces cerevisiae. An exemplary alcohol-O-acyltransferase is ScATF1 from Saccharomyces cerevisiae, as described, for example, in Verstrepen K J, et al. Appl Microbiol Biotechnol. (2003) 61(3):197-205. The amino acid sequence of ScATF1 from Saccharomyces cerevisiae is set forth as SEQ ID NO: 12.

(SEQ ID NO: 12)
MNEIDEKNQAPVQQECLKEMIQNGHARRMGSVEDLYVALNRQNLYRNFCTYGELSDYCTRDQLTLALREICLKNP
TLLHIVLPTRWPNHENYYRSSEYYSRPHPVHDYISVLQELKLSGVVLNEQPEYSAVMKQILEEFKNSKGSYTAKI
FKLTTILTIPYFGPTGPSWRLICLPEEHTEKWKKFIFVSNHCMSDGRSSIHFFHDLRDELNNIKTPPKKLDYIFK
YEEDYQLLRKLPEPIEKVIDFRPPYLFIPKSLLSGFIYNHLRFSSKGVCMRMDDVEKIDDVVTEIINISPTEFQA
IKANIKSNIQGKCTITPFLHVCWFVSLHKWGKFFKPLNFEWLTDIFIPADCRSQLPDDDEMRQMYRYGANVGFID
FTPWISEFDMNDNKENFWPLIEHYHEVISEALRNKKHLHGLGFNIQGFVQKYVNIDKVMCDRAIGKRRGGTLLSN
VGLFNQLEEPDAKYSICDLAFGQFQGSWHQAFSLGVCSTNVKGMNIVVASTKNVVGSQESLEELCSIYKALLLGP

In some embodiments, the alcohol-O-acyltransferase is from Neurospora sitophila. An exemplary alcohol-O-acyltransferase is NsATF1 from Neurospora sitophila, and the amino acid sequence of which is set forth as SEQ ID NO: 13.

(SEQ ID NO: 13)
MGTSIPQPIRPLGPCEAYSSSRHALGFYRCLANTCRYAVPWSVLQGKSVPDVLEAAIANLVLRLPRLSVAITGDE
ASRPYFASVSSLDLSYHLECVELRAELDFHARDSHLLHMLEAQHNQLWPDVGFRPPWKVLAVYDPRPSQLEDRLI
LDIVLAIHHSLADGRSTAIFQTSLLDELNKPPVRPSCLEDHVLRMPSKPHGHILPPQEELVKFTTSWRFLAGTLW
NEFVSGWLYKPATDLPWAGAPIRPDPYQTRLRLVTIPAKAVSQLLINCRANETTLTPLLHVLILTSLARRLTAEA
ATSFQSCTPVDLRPFIQSGSHVADPAEVFGVLVTSASHSFNSSRVSGLREQASGEKIWSLAQTLRQELKDRLEAI
PQDDMVSMLRWIANWRGFWLNKVNKPREHTLEVSNIGSLHGSPEKTANADLETGSKWQIVRSVMSQCAIVAGPAL
CASVSGVVGGPISIALSWQEGIIESELVEGVAHDLQLWMNQGGPVHGQRLP

In some embodiments, the alcohol-O-acyltransferase is from Actinidia deliciosa. An exemplary alcohol-O-acyltransferase is AdAAT1 from Actinidia deliciosa, as described, for example, in Gunther C S, et al. Phytochemistry (2011) 72(8): 700-10. In some embodiments, the amino acid sequence of AdAAT1 from Actinidia deliciosa is set forth as SEQ ID NO: 15.

(SEQ ID NO: 15)
MASSVRLVKKPVLVAPVDPTPSTVLSLSSLDSQLFLRFPIEYLLVYASPHGVDRAVTAARVKAALARSLVPYYPL
AGRVKTRPDSTGLDVVCQAQGAGLLEAVSDYTASDFQRAPRSVTEWRKLLLVEVFKVVPPLVVQLTWLSDGCVAL
GVGFSHCVIDGIGSSEFLNLFAELATGRARLSEFQPKPVWDRHLLNSAGRINLGTHPEFGRVPDLSGFVTRFTQE
RLSPTSITFDKTWLKELKNIAMSTSQPGEFPYTSFEVLSGHIWRSWARSLNLPAKQVLKLLFSINIRNRVKPSLP
AGYYGNAFVLGCAQTSVKDLTEKGLGYCADLVRGAKERVGDEYAREVVESVSWPRRASPDSVGVLIISQWSRLGL
DRVDFGLGRPVQVGPICCDRYCLFLPVRDRTESVKVMVAVPTSAVDRYEYFIRSPYS

In some embodiments, the alcohol-O-acyltransferase is from Actinidia chinensis. An exemplary alcohol-O-acyltransferase is AcAAT16 from Actinidia chinensis, as described, for example, in Gunther C S, et al. Phytochemistry (2011) 72(8): 700-10. In some embodiments, the amino acid sequence of AcAAT16 from Actinidia chinensis is set forth as SEQ ID NO: 16.

(SEQ ID NO: 16)
MASFPPSLVFTVRRNEPTLVLPSKSTPRELKQLSDIDDQEGLRFQVPVIM
FYKRKLSMEGEDPVKVIREALAEALVFYYPFAGRLIEGPNRKLMVDCTGE
GVLFIEADADIEVNQLIGDTIDPGFSYLDELLHDVPGSEGILGCPLLLIQ
VTRFRCGGWAFAIRLNHTMSDAPGLVQLLTTIAEFARGAEGAPSVPPVWQ
REFLAARQPPSITFQHHEYEQVINTTTDDNKSMTHKSFFFGPKEIRAIRS
HFPPHYRSVSSTEDVLTACLWRCRICALGLDPPKTVRISCAANGRGKHDL
HVPRGYYGNVFAFPAVVSRAGMISTSSLEYTVEEVKKAKARMIGEYLRSV
ADLMVTKGRPLYTVAGNYIVSDTTRVGFDAIDFGWGKPVYGGPARAFPLI
SFYARFKNNRGEDGTVVLICLPEAAMKRFQDELKKMTEEHVDGPFEYKLI
KVMSKL

In some embodiments, the alcohol-O-acyltransferase is from Saccharomycopsis fibuligera. An exemplary alcohol-O-acyltransferase is SfATFA2 from Saccharomycopsis fibuligera, as described, for example, in Moon HY, et al. Systems and Synthetic Microbiology and Bioinformatics (2021) 59, 598-608. In some embodiments, the amino acid sequence of SfATFA2 from Saccharomycopsis fibuligera is set forth as SEQ ID NO: 17.

(SEQ ID NO: 17)
MTSETLQTSSSSFPASEASQKDSTPAQTTQTAQKQGPVKSKDDLTYKAPF
LERNFYFSSKHELFNCFGVSIVVNKPISREQFYVALRKIILKYPKSITSV
YDEFDREHHLRFIPKTKIIFDDNAVEFNEKFDQYPYQNKELSALLTSYRF
DADPNNGKPSWKIVYFPKIKMLSWLFDHPISDGASGAAFCKELVESLNYI
TQKELDEAKDLFESSAANKKAVELFNLEKDISKFENPITPDSFKIAGYKP
SLAEKIGTPILRFFLDKFPKLFPLVIEGEMHKQQFVDTKPIKFDNKKFFV
REQDVISKDSPLCGQALTYIRIDPETTAKILQQCRNNNTKFQTTFMMVFL
STIHEIAPEAYINKYLKIVTAANFRHIFPNYKYGHSKFLSKPDSYTKETG
QFKDGFHDHAVVFYVEPFKKENWNLVQKYHNFLHKLIRSKQWFSGYYLAS
EAVSAKTFFDQKIGTHDDTYFALTNLGFVDLIDHGEEASNKYQIEDLIFT
ASPGPMIGTHSAVLTSTKNGINICVADQDPAINSEEFRARLTENLRKLAE
SGNV

An exemplary alcohol-O-acyltransferase is SfATFB4 from Saccharomycopsis fibuligera, as described, for example, in Moon H Y, et al. Systems and Synthetic Microbiology and Bioinformatics (2021) 59, 598-608. In some embodiments, the amino acid sequence of SfATFB4 from Saccharomycopsis fibuligera is set forth as SEQ ID NO: 18.

(SEQ ID NO: 18)
MGNFQFSRNDFYTDPTFTEKCFYYYDQYGLISNFSVTIKTTASITRELLY
AALKKVILKYPNLVSSIHDKFDYDTHNEKTLIKSPKKIIYFDDNIVQFIS
QDEETRNYADINQIQLLLNATKFDSNFTNGKPMWKIFVFPNKNLTSWVED
YSIFDGGSAIVYQKELVEALNQILESEQQKAREILDNASKRTTPILFDFE
KDWPLFQRAPSQGIFKEINYVPSIFKKVSSQVIKLLSNAVPDKTIDELND
EANKSAFLERIIFEKEKLYLSKNVIGLESGAAKPLSKIININHIILSKIL
DKCHTKGCNFQAIFIIIFLATVHQVIPLQYSKKYLKTVTSASFRNIFTKQ
FVSHNEYLAEQELGIQKLLQGQQQFIDGIFVHSAIIYIEPFDEFSWELCH
KYDSFLHILLHSKGWFANYYVANRGIQAKAFVDNKLGSQDDVFVSFDNLG
LVRVKESGKFQIEDIIFTKAPDPIAGDNLIAMVSTKKGGLNIQINIAEEH
IQARFDEFCLRLSENLIALGNF

In some embodiments, the alcohol-O-acyltransferase is from Malus x domestica. An exemplary alcohol-O-acyltransferase is MpAAT1 from Malus x domestica, as described, for example, in Dunemann F, et al. Molecular Breeding (2012) 29, 609-625.

In some embodiments, the amino acid sequence of MpAAT1 from Malus x domestica is set forth as SEQ ID NO: 19.

(SEQ ID NO: 19)
MMSFSVLQVKRLQPELITPAKSTPQETKFLSDIDDQESLRVQIPIIMCYK
DNPSLNKNRNPVKAIREALSRALVYYYPLAGRLREGPNRKLVVDCNGEGI
LFVEASADVTLEQLGDKILPPCPLLEEFLYNFPGSDGIIDCPLLLIQVTC
LTCGGFILALRLNHTMCDAAGLLLFLTAIAEMARGAHAPSILPVWERELL
FARDPPRITCAHHEYEDVIGHSDGSYASSNQSNMVQRSFYFGAKEMRVLR
KQIPPHLISTCSTFDLITACLWKCRTLALNINPKEAVRVSCIVNARGKHN
NVRLPLGYYGNAFAFPAAISKAEPLCKNPLGYALELVKKAKATMNEEYLR
SVADLLVLRGRPQYSSTGSYLIVSDNTRVGFGDVNFGWGQPVFAGPVKAL
DLISFYVQHKNNTEDGILVPMCLPSSAMERFQQELERITQEPKEDICNNL
RSTSQ

In some embodiments, the alcohol-O-acyltransferase is from Marinobacter hydrocarbonoclasticus. An exemplary alcohol-O-acyltransferase is MhWES2 from Marinobacter hydrocarbonoclasticus, as described by Holtzeapple E, et al. Journal of Bacteriology (2007) 189: 10. In some embodiments, the alcohol-O-acyltransferase is MhWES2 from Marinobacter hydrocarbonoclasticus and comprises one or more mutations (e.g., substitutions, insertions, deletions). In some embodiments, the alcohol-O-acyltransferase is MhWES2 from Marinobacter hydrocarbonoclasticus and does not comprise a glycine (G) residue at position 150. In some embodiments, the alcohol-O-acyltransferase is MhWES2 from Marinobacter hydrocarbonoclasticus and comprises a phenylalanine (F) residue at position 150. The amino acid sequence of MhWES2 from Marinobacter hydrocarbonoclasticus comprising a phenylalanine at the position corresponding to 150 is set forth as SEQ ID NO: 21.

(SEQ ID NO: 21)
MGKRLGILDASWLAVESEDTPMHVGTLQIFSLPEGAPETFLRDMVTRMKE
AGDVAPPWGYKLAWSGFLGRVIAPAWKVDKDIDLDYHVRHSALPRPGGER
ELGILVSRLHSNPLDFSRPLWECHVIEGLENNRFALYTKMHHSMIDGISF
VRLMQRVLTTDPERCNMPPPWTVRPHQRRGAKTDKEASVPAAVSQAMDAL
KLQADMAPRLWQAGNRLVHSVRHPEDGLTAPFTGPVSVLNHRVTAQRRFA
TQHYQLDRLKNLAHASGGSLNDIVLYLCGTALRRFLAEQNNLPDTPLTAG
IPVNIRPADDEGTGTQISFMIASLATDEADPLNRLQQIKTSTRRAKEHLQ
KLPKSALTQYTMLLMSPYILQLMSGLGGRMRPVFNVTISNVPGPEGTLYY
EGARLEAMYPVSLIAHGGALNITCLSYAGSLNFGFTGCRDTLPSMQKLAV
YTGEALDELESLILPPKKRARTRK

Amino acids of the alcohol-O-acyltransferase may be modified (e.g., substituted) to produce an alcohol-O-acyltransferase variant. For example, as described herein, the amino acid at position 144 and/or 360, referred to as alanine 144 and alanine 360, respectively, of SEQ ID NO: 1 may be mutated to produce an alcohol-O-acyltransferase enzyme having a desired activity, such as increased production of ethyl-hexanoate during fermentation, increased production of hexanoic acid during fermentation, and/or increased ratio of ethyl-hexanoate to hexanoic acid production. In some embodiments the amino acid corresponding to alanine 144 and/or alanine 360 of SEQ ID NO: 1 is substituted with an amino acid that is not an alanine residue (e.g., any other amino acid).

In some embodiments, the amino acid corresponding to alanine at position 144 (A144) of SEQ ID NO: 1 is substituted with an amino acid selected from histidine (H), arginine (R), lysine (K), aspartic acid (D), glutamic acid (E), serine (S), threonine (T), asparagine (N), glutamine (G), cysteine (C), glycine (G), proline (P), valine (V), isoleucine (I), leucine (L), methionine (M), phenylalanine (F), tyrosine (Y), or tryptophan (W). In some embodiments, the amino acid corresponding to alanine at position 144 (A144) of SEQ ID NO: 1 is substituted with a hydrophobic amino acid (e.g., histidine (H), valine (V), isoleucine (I), leucine (L), methionine (M), phenylalanine (F), tyrosine (Y), tryptophan (W)). In some embodiments, the amino acid corresponding to alanine at position 144 (A144) of SEQ ID NO: 1 is substituted with a phenylalanine (F) residue (A144F).

In some embodiments, the amino acid corresponding to alanine at position 360 (A360) of SEQ ID NO: 1 is substituted with an amino acid selected from histidine (H), arginine (R), lysine (K), aspartic acid (D), glutamic acid (E), serine (S), threonine (T), asparagine (N), glutamine (G), cysteine (C), glycine (G), proline (P), valine (V), isoleucine (I), leucine (L), methionine (M), phenylalanine (F), tyrosine (Y), or tryptophan (W). In some embodiments, the amino acid corresponding to alanine at position 360 (A360) of SEQ ID NO: 1 is substituted with a hydrophobic amino acid (e.g., histidine (H), valine (V), isoleucine (I), leucine (L), methionine (M), phenylalanine (F), tyrosine (Y), tryptophan (W)). In some embodiments, the amino acid corresponding to alanine at position 360 (A360) of SEQ ID NO: 1 is substituted with an isoleucine (I) residue (A360I).

In some embodiments, the amino acid corresponding to alanine at position 144 (A144) of SEQ ID NO: 1 is substituted with a phenylalanine (F) residue (A144F) and the amino acid corresponding to alanine at position 360 (A360) of SEQ ID NO: 1 is substituted with an isoleucine (I) residue (A360I), provided by SEQ ID NO: 4.

Amino acid sequence of variant MaWES from Marinobacter hydrocarbonoclasticus-A144F and A360I mutations (A144F and A360I)

(SEQ ID NO: 4)
MTPLNPTDQLFLWLEKRQQPMHVGGLQLFSFPEGAPDDYVAQLADQLRQK
TEVTAPFNQRLSYRLGQPVWVEDEHLDLEHHFRFEALPTPGRIRELLSFV
SAEHSHLMDRERPMWEVHLIEGLKDRQFALYTKVHHSLVDGVSFMRMATR
MLSENPDEHGMPPIWDLPCLSRDRGESDGHSLWRSVTHLLGLSGRQLGTI
PTVAKELLKTINQARKDPAYDSIFHAPRCMLNQKITGSRRFAAQSWCLKR
IRAVCEAYGITVNDVVTAMCAAALRTYLMNQDALPEKPLVAFVPVSLRRD
DSSGGNQVGVILASLHTDVQEAGERLLKIHHGMEEAKQRYRHMSPEEIVN
YTALTLAPAIFHLLTGLAPKWQTENVVISNVPGPSRPLYWNGAKLEGMYP
VSIDMDRLALNMTLTSYNDQVEFGLIGCRRTLPSLQRMLDYLEQGLAELE
LNAGL

In some embodiments, the alcohol-O-acyltransferase is from Malus x domestica, or a variant thereof. An exemplary alcohol-O-acyltransferase is MpAAT1 from Malus x domestica, as described, for example, in Dunemann F. et al. Molecular Breeding (2012) 29, 609-625. In some embodiments, the alcohol-O-acyltransferase is MpAAT1 from Malus x domestica and comprises one or more mutations (e.g., substitutions, insertions, deletions).

In some embodiments, the amino acid corresponding to alanine at position 169 (A169) of SEQ ID NO: 19 is substituted with an amino acid selected from histidine (H), arginine (R), lysine (K), aspartic acid (D), glutamic acid (E), serine (S), threonine (T), asparagine (N), glutamine (Q), cysteine (C), glycine (G), proline (P), valine (V), isoleucine (I), leucine (L), methionine (M), phenylalanine (F), tyrosine (Y), or tryptophan (W). In some embodiments, the amino acid corresponding to alanine at position 169 (A169) of SEQ ID NO: 19 is substituted with a glycine (G) residue (A169G).

In some embodiments, the amino acid corresponding to alanine at position 170 (A170) of SEQ ID NO: 19 is substituted with an amino acid selected from histidine (H), arginine (R), lysine (K), aspartic acid (D), glutamic acid (E), serine (S), threonine (T), asparagine (N), glutamine (Q), cysteine (C), glycine (G), proline (P), valine (V), isoleucine (I), leucine (L), methionine (M), phenylalanine (F), tyrosine (Y), or tryptophan (W). In some embodiments, the amino acid corresponding to alanine at position 170 (A170) of SEQ ID NO: 19 is substituted with a phenylalanine (F) residue (A170F).

In some embodiments, the alcohol-O-acyltransferase is MpAAT1 from Malus x domestica and comprises a glycine (G) at residue 169 and a phenylalanine at residue 170 relative to SEQ ID NO: 19. The amino acid sequence of MpAAT1 from Malus x domestica comprising a glycine at residue 169 and a phenylalanine at residue 170 is set forth as SEQ ID NO: 20.

(SEQ ID NO: 20)
MMSFSVLQVKRLQPELITPAKSTPQETKFLSDIDDQESLRVQIPIIMCYK
DNPSLNKNRNPVKAIREALSRALVYYYPLAGRLREGPNRKLVVDCNGEGI
LFVEASADVTLEQLGDKILPPCPLLEEFLYNFPGSDGIIDCPLLLIQVTC
LTCGGFILALRLNHTMCDGFGLLLFLTAIAEMARGAHAPSILPVWERELL
FARDPPRITCAHHEYEDVIGHSDGSYASSNQSNMVQRSFYFGAKEMRVLR
KQIPPHLISTCSTFDLITACLWKCRTLALNINPKEAVRVSCIVNARGKHN
NVRLPLGYYGNAFAFPAAISKAEPLCKNPLGYALELVKKAKATMNEEYLR
SVADLLVLRGRPQYSSTGSYLIVSDNTRVGFGDVNFGWGQPVFAGPVKAL
DLISFYVQHKNNTEDGILVPMCLPSSAMERFQQELERITQEPKEDICNNL
RSTSQ

In some embodiments, the enzyme comprises the amino acid sequence of any one of SEQ ID NOs: 1-4 and 12-22. In some embodiments, the enzyme comprises the amino acid sequence of any one of SEQ ID NOs: 1-3, wherein the amino acid corresponding to alanine at position 144 (A144) and/or the amino acid corresponding to alanine at position 360 (A360), based on the reference sequence provided by SEQ ID NO: 1, is substituted with a hydrophobic amino acid (e.g., histidine (H), valine (V), isoleucine (I), leucine (L), methionine (M), phenylalanine (F), tyrosine (Y), tryptophan (W)). In some embodiments, the amino acid corresponding to position 144 (A144) is substituted with a phenylalanine (F) and/or the amino acid corresponding to position 360 (A360) is substituted with an isoleucine (I).

In some embodiments, the heterologous gene encodes an enzyme with alcohol-O-acyltransferase activity such that a cell that expresses the enzyme is capable of increased production of ethyl-hexanoate as compared to a cell that does not express the heterologous gene. In some embodiments, the heterologous gene encodes an enzyme with alcohol-O-acyltransferase activity such that a cell that expresses the enzyme is capable of producing increased levels of ethyl-hexanoate as compared to a cell that expresses an enzyme with wild-type alcohol-O-acyltransferase activity. In some embodiments, the heterologous gene encodes an enzyme with alcohol-O-acyltransferase activity such that a cell that expresses the enzyme is capable of producing reduced levels of hexanoic acid as compared to a cell that does not express the heterologous gene. In some embodiments, the heterologous gene encodes an enzyme with alcohol-O-acyltransferase activity such that a cell that expresses the enzyme is capable of producing reduced levels of hexanoic acid as compared to a cell that expresses an enzyme with wild-type alcohol-O-acyltransferase activity. In some embodiments, the enzyme with alcohol-O-acyltransferase activity that is capable of producing increased levels of ethyl-hexanoate contains a substitution of the amino acid at the position corresponding to alanine at position 144 (A144) and/or alanine at position 360 (A360) of SEQ ID NO: 1. In some embodiments, the enzyme with alcohol-O-acyltransferase activity that is capable of producing increased levels of ethyl-hexanoate has the sequence provided by any one of SEQ ID NOs: 2-4 and 12-22.

In some embodiments, the enzyme with alcohol-O-acyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in any one of SEQ ID NOs: 1-4 and 12-22. In some embodiments, the enzyme with alcohol-O-acyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in any one of SEQ ID NOs: 1-4 and 12-22, and the amino acid corresponding to alanine at position 144 (A144) of SEQ ID NO: 1 and/or the amino acid corresponding to alanine at position 360 (A360) of SEQ ID NO: 1 is substituted with an amino acid that is not an alanine residue (e.g., any other amino acid). In some embodiments, the enzyme with alcohol-O-acyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in any one of SEQ ID NOs: 1-4 and 12-22, and the amino acid corresponding to alanine at position 144 (A144) of SEQ ID NO: 1 and/or the amino acid corresponding to alanine at position 360 (A360) of SEQ ID NO: 1 is substituted with an amino acid selected from histidine (H), arginine (R), lysine (K), aspartic acid (D), glutamic acid (E), serine (S), threonine (T), asparagine (N), glutamine (G), cysteine (C), glycine (G), proline (P), valine (V), isoleucine (I), leucine (L), methionine (M), phenylalanine (F), tyrosine (Y), or tryptophan (W).

The terms “percent identity,” “sequence identity,” “% identity,” “% sequence identity,” and % identical,” as they may be interchangeably used herein, refer to a quantitative measurement of the similarity between two sequences (e.g., nucleic acid or amino acid). Percent identity can be determined using the algorithms of Karlin and Altschul, Proc. Natl. Acad. Sci. USA 87:2264-68, 1990, modified as in Karlin and Altschul, Proc. Natl. Acad. Sci. USA 90:5873-77, 1993. Such algorithms are incorporated into the NBLAST and XBLAST programs (version 2.0) of Altschul et al., J. Mol. Biol. 215:403-10, 1990. BLAST protein searches can be performed with the XBLAST program, score=50, word length=3, to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5% ,at least 98%, at least 98.5%, at least 99%, at least 99.5%, at least 99.6%, at least 99.7%, at least 99.8%, or at least 99.9% identity) and all increments thereof (e.g., tenths of a percent (i.e., 0.1%), hundredths of a percent (i.e., 0.01%), etc.).

In some embodiments, the enzyme with alcohol-O-acyltransferase activity comprises an amino acid sequence as set forth in SEQ ID NO: 1. In some embodiments, the enzyme with alcohol-O-acyltransferase activity consists of the amino acid sequence as set forth in SEQ ID NO: 1. In some embodiments, the enzyme with alcohol-O-acyltransferase activity comprises an amino acid sequence as set forth in SEQ ID NO: 2. In some embodiments, the enzyme with alcohol-O-acyltransferase activity consists of the amino acid sequence as set forth in SEQ ID NO: 2. In some embodiments, the enzyme with alcohol-O-acyltransferase activity comprises an amino acid sequence as set forth in SEQ ID NO: 3. In some embodiments, the enzyme with alcohol-O-acyltransferase activity consists of the amino acid sequence as set forth in SEQ ID NO: 3. In some embodiments, the enzyme with alcohol-O-acyltransferase activity comprises an amino acid sequence as set forth in SEQ ID NO: 4. In some embodiments, the enzyme with alcohol-O-acyltransferase activity consists of the amino acid sequence as set forth in SEQ ID NO: 4.

In some embodiments, the enzyme with alcohol-O-acyltransferase activity consists of the amino acid sequence as set forth in SEQ ID NO: 12. In some embodiments, the enzyme with alcohol-O-acyltransferase activity consists of the amino acid sequence as set forth in SEQ ID NO: 13. In some embodiments, the enzyme with alcohol-O-acyltransferase activity consists of the amino acid sequence as set forth in SEQ ID NO: 14. In some embodiments, the enzyme with alcohol-O-acyltransferase activity consists of the amino acid sequence as set forth in SEQ ID NO: 15. In some embodiments, the enzyme with alcohol-O-acyltransferase activity consists of the amino acid sequence as set forth in SEQ ID NO: 16. In some embodiments, the enzyme with alcohol-O-acyltransferase activity consists of the amino acid sequence as set forth in SEQ ID NO: 17. In some embodiments, the enzyme with alcohol-O-acyltransferase activity consists of the amino acid sequence as set forth in SEQ ID NO: 18. In some embodiments, the enzyme with alcohol-O-acyltransferase activity consists of the amino acid sequence as set forth in SEQ ID NO: 19. In some embodiments, the enzyme with alcohol-O-acyltransferase activity consists of the amino acid sequence as set forth in SEQ ID NO: 20. In some embodiments, the enzyme with alcohol-O-acyltransferase activity consists of the amino acid sequence as set forth in SEQ ID NO: 21.

In some embodiments, the gene encoding the enzyme with alcohol-O-acyltransferase activity comprises a nucleic acid sequence which encodes an enzyme comprising an amino acid sequence with at least 80% (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9%) sequence identity to the sequence as set forth in any one of SEQ ID NOs: 1-4 and 12-22. In some embodiments, the gene encoding the enzyme with alcohol-O-acyltransferase activity comprises a nucleic acid sequence which encodes an enzyme consisting of an amino acid sequence as set forth in any one of SEQ ID NOs: 1-4 and 12-22.

Identification of additional enzymes having alcohol-O-acyltransferase activity or predicted to have alcohol-O-acyltransferase activity may be performed, for example based on similarity or homology with one or more domains of an alcohol-O-acyltransferase, such as the alcohol-O-acyltransferase provided by any one of SEQ ID NOs: 1-4 and 12-22. In some embodiments, an enzyme for use in the modified cells and methods described herein may be identified based on similarity or homology with an active domain, such as a catalytic domain, such as a catalytic domain associated with alcohol-O-acyltransferase activity. In some embodiments, an enzyme for use in the modified cells and methods described herein may have a relatively high level of sequence identity with a reference alcohol-O-acyltransferase, e.g., a wild-type alcohol-O-acyltransferase, such as any of SEQ ID NOs: 1, 2, 3, 12, 13, 14, 16, 17, 18, 19, or 22, in the region of the catalytic domain but a relatively low level of sequence identity to the reference alcohol-O-acyltransferase based on analysis of a larger portion of the enzyme or across the full length of the enzyme. In some embodiments, the enzyme for use in the modified cells and methods described herein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9% sequence identity in the region of the catalytic domain of the enzyme relative to a reference alcohol-O-acyltransferase (e.g., SEQ ID NO: 1).

In some embodiments, the enzymes for use in the modified cells and methods described herein have a relatively high level of sequence identity in the region of the catalytic domain of the enzyme relative to a reference alcohol-O-acyltransferase (e.g., any of SEQ ID NOs: 1-3) and a relatively low level of sequence identity to the reference alcohol-O-acyltransferase based on analysis of a larger portion of the enzyme or across the full length of the enzyme. In some embodiments, the enzymes for use in the modified cells and methods described herein have at least 10%, at least 15%, at least 20%, at least 25%, least 30% at least 35%, at least 40%, at least 45%, at least 50%, at least 55%, at least 60%, at least 65%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9% sequence identity based on a portion of the enzyme or across the full length of the enzyme relative to a reference alcohol-O-acyltransferase (e.g., SEQ ID NOs: 1-4, 12-19, and 21-22).

In some embodiments, the amino acid substitution(s) may be in the active site. As used herein, the term “active site” refers to a region of the enzyme with which a substrate interacts. The amino acids that comprise the active site and amino acids surrounding the active site, including the functional groups of each of the amino acids, may contribute to the size, shape, and/or substrate accessibility of the active site. In some embodiments, the alcohol-O-acyltransferase variant contains one or more modifications that are substitutions of a selected amino acid with an amino acid having a different functional group.

This information can also be used to identify positions, e.g., corresponding positions, in other enzymes having or predicted to have alcohol-O-acyltransferase activity. As will be evident to one of ordinary skill in the art, an amino acid substitution at a position identified in one alcohol-O-acyltransferase enzyme can also be made in the corresponding amino acid position of another alcohol-O-acyltransferase enzyme. In such instances, one of the alcohol-enzymes may be used as a reference enzyme. For example, as described herein, amino acid substitutions at position A144 and/or A360 of MaWES from Marinobacter aquaeolei (SEQ ID NO: 1) have been shown to increase production of ethyl-hexanoate and/or reduce production of hexanoic acid. Similar amino acid substitutions can be made at the corresponding position of other enzymes having alcohol-O-acyltransferase activity using MaWES as a reference (e.g., SEQ ID NO: 1). For example, amino acid substitutions can be made at the corresponding position(s) of an alcohol-O-acyltransferase from F. ananassa or S. lycopersicum, as described herein, using MaWES as a reference (e.g., SEQ ID NO: 1). In some embodiments, the amino acid at the position corresponding to position A144 and/or A360 of MaWES from M. hydrocarbonoclasticus (SEQ ID NO: 1) in another enzyme (e.g., an alcohol-O-acyltransferase from F. ananassa (see, e.g,. SEQ ID NO: 2) is not an alanine. In some embodiments, the amino acid at the position corresponding to position A144 and/or A360 of MaWES from M. hydrocarbonoclasticus (SEQ ID NO: 1) in another enzyme (e.g., an alcohol-O-acyltransferase from S. lycopersicum (see, e.g,. SEQ ID NO: 3) is not an alanine.

The alcohol-O-acyltransferase variants described herein contain an amino acid substitution of one or more positions corresponding to a reference alcohol-O-acyltransferase. In some embodiments, the alcohol-O-acyltransferase variant contains an amino acid substitution at 1, 2, 3, 4, 5, or more positions corresponding to a reference alcohol-O-acyltransferase. In some embodiments, the alcohol-O-acyltransferase is not a naturally occurring alcohol-O-acyltransferase, e.g., is genetically modified. In some embodiments, the alcohol-O-acyltransferase does not have the amino acid sequence provided by SEQ ID NO: 1.

The genetically modified cells described herein contain, in some embodiments, genetic modifications that reduce the expression and/or activity of endogenous genes encoding enzymes with alcohol-O-acyltransferase (AAT) activity. The term “endogenous gene,” as used herein, refers to a hereditary unit corresponding to a sequence of nucleic acid (e.g., DNA) that contains the genetic instruction, which originates within a host organism (e.g., a genetically modified cell) and is expressed by the host organism.

The Saccharomyces cerevisiae yeast genome encodes at least seven alcohol-O-transferases that are thought to have redundant ester and acyl-CoA hydrolysis activities. Non-limiting examples of endogenous S. cerevisiae genes encoding enzymes having alcohol-O-acyltransferase activity include Atf1p, Atf2p, Eat1p, Eht1p, Eeb1p, Iah1p, and Mgl2p, and corresponding protein products ATF1, ATF2, EAT1, EHT1, EEB1, IAH1, and MGL2. In some embodiments, the modified cells do not express endogenous Eeb1p or EEB1. Methods of reducing expression and/or activity of a desired gene are well known in the art. For example, the promoter controlling expression of the endogenous gene may be modified to be less permissive to transcription initiation, resulting in reduced transcription and thus less protein production and lower enzyme activity in the modified cell. Alternatively, the epigenome may be methylated or otherwise modified to inhibit transcription, resulting in reduced protein production and consequently lower enzyme activity in the modified cell.

In some embodiments, an endogenous gene encoding one or more alcohol-O-acyltransferases are deleted from the genome of modified cells. Methods of deleting a gene from the genome of an organism are well known in the art. For example, a DNA construct encoding a non-functional gene or alternatively a reporter or drug resistance gene, flanked by DNA sequences that correspond to the 5′ and 3′ regions that flank the endogenous gene in the genome, may be introduced to a target cell, where it may be integrated into the targeted region of the by homologous recombination. In some embodiments, one or more endogenous genes encoding one or more alcohol-O-acyltransferase are deleted from the genome of the modified cells. In some embodiments, the Eeb1p gene, or a portion thereof, is replaced by homologous recombination. In some embodiments, following recombination, the genome of the cell does not contain an intact Eeb1p gene, and the cell is thus deficient in EEB1 activity. In some embodiments, the Eht1 gene, or a portion thereof, is replaced by homologous recombination. In some embodiments, following recombination, the genome of the cell does not contain an intact Eht1 gene, and the cell is thus deficient in EHT1 activity. In some embodiments, the Mgl2 gene, or a portion thereof, is replaced by homologous recombination.

In some embodiments, following recombination, the genome of the cell does not contain an intact Mgl2 gene, and the cell is thus deficient in MGL2 activity. In some embodiments, the Eht1 gene and the Eeb1p gene, or a portion thereof, is replaced by homologous recombination. In some embodiments, following recombination, the genome of the cell does not contain an intact Eht1 gene or Eeb1p gene, and the cell is thus deficient in EHT1 and EEB1 activity. In some embodiments, the Eht1 gene and the Mgl2 gene, or a portion thereof, is replaced by homologous recombination. In some embodiments, following recombination, the genome of the cell does not contain an intact Eht1 gene or Mgl2 gene, and the cell is thus deficient in EHT1 and MGL2 activity. In some embodiments, the Eeb1p gene and the Mgl2 gene, or a portion thereof, is replaced by homologous recombination. In some embodiments, following recombination, the genome of the cell does not contain an intact Eeb1p gene or Mgl2 gene, and the cell is thus deficient in EEB1 and MGL2 activity. In some embodiments, the Eeb1p gene, the Eht1 gene, and the Mgl2 gene, or a portion thereof, is replaced by homologous recombination. In some embodiments, following recombination, the genome of the cell does not contain an intact Eeb1p gene, Eht1 gene, or the Mgl2 gene, and the cell is thus deficient in EEB1, EHT1 and MGL2 activity.

In some embodiments, an endogenous gene encoding one or more alcohol-O-acyltransferases are modified to reduce alcohol-O-acyltransferase activity. For example, one or more mutation may be made in endogenous gene encoding an alcohol-O-acyltransferase (e.g., one or more mutations in any of Eeb1p, Eht1, and/or Mgl2), such that the enzyme has reduced or eliminated alcohol-O-acyltransferase activity.

### Fatty Acid Synthase 2 (FAS2) Enzymes

The genetically modified cells described herein contain a gene encoding an enzyme with fatty acid synthase (FAS2) activity. In some embodiments, the gene is an exogenous gene. The term “exogenous gene,” as used herein, refers to a hereditary unit corresponding to a sequence of nucleic acid (e.g., DNA) that contains the genetic instruction, which is introduced into a host organism (e.g., a genetically modified cell) from an external source, and expressed by the host organism. In some embodiments, the exogenous gene is a further copy of a gene that is present in the cell.

The metabolites produced during fermentation can impart distinctive flavors to a fermented beverage. As discussed herein, ethyl-hexanoate, for example, is a fatty acid ester that imparts a pineapple flavor. However, hydrolysis of the ester bond of ethyl-hexanoate results in the formation of ethanol and hexanoic acid, a pungent fatty acid that imparts cheesy, rancid, and goaty flavors when present at a concentration above a flavor detection threshold. Accordingly, producing hexanoic acid during production of a fermented product intended for consumption is undesirable, as beverages containing hexanoic acid concentrations above a flavor detection threshold are widely considered undrinkable and are not commercially viable. Thus, to produce fermented beverages that are considered palatable and commercially viable, compositions and methods for increasing ethyl-hexanoate production during fermentation must do so while minimizing the production of hexanoic acid to a level below the flavor detection threshold.

The fatty acid synthetase complex contains 6 polypeptide α subunits (encoded by FAS2) and 6 polypeptide ⊕ subunits (encoded by FAS1). The α subunit, referred to herein as “FAS2,” is thought to be involved in the extension of fatty acid chains and affect production of hexanoyl-CoA, which may be used to form both ethyl-hexanoate and hexanoic acid during fermentation.

The genetically modified cells described herein may express a gene, such as an exogenous gene, encoding an enzyme having fatty acid synthase (FAS2) activity. In some embodiments, the enzyme having fatty acid synthase (FAS2) activity is obtained from a bacterium or a fungus. In some embodiments, the enzyme having fatty acid synthase (FAS2) activity is obtained from a yeast. In some embodiments, the enzyme having fatty acid synthase (FAS2) activity is from a Saccharomyces species. In some embodiments, the enzyme having fatty acid synthase (FAS2) activity is from Saccharomyces cerevisiae.

An exemplary enzyme having fatty acid synthase activity is FAS2 from Saccharomyces cerevisiae WLP001, which is provided by the amino acid sequence set forth as SEQ ID NO: 5.

(SEQ ID NO: 5)
MKPEVEQELAHILLTELLAYQFASPVRWIETQDVFLKDENTERVVEIGPS
PTLAGMAQRTLKNKYESYDAALSLHREILCYSKDAKEIYYTPDPSELAAK
EEPAKEEAPAPTPAASAPAPAAAAPAPVAAAAPAAAAAEIADEPVKASLL
LHVLVAHKLKKSLDSIPMSKTIKDLVGGKSTVQNEILGDLGKEFGTTPEK
SEETPLEELAETFQDTFSGALGKQSSSLLSRLISSKMPGGFTITVARKYL
QTRWGLPSGRQDGVLLVALSNEPAARLGSEADAKAFLGSMAQKYASIVGV
DLSSAASASGAAGAGAAAGAAMIDAGALEEITKDHKVLARQQLQVLARYL
KMDLDNGERKFLKEKDTVAELQAQLDYLNAELGEFFVNGVATSFSRKKAR
TFDSSWNWAKQSLLSLYFEIIHGVLKNVDREVVSEAINIMNRSNDALIKF
MEYHISNTDETKGENYQLVKTLGEQLIENCKQVLDVDPVYKDVAKPTGPK
TAIDKNGNITYSEEPREKVRKLSQYVQEMALGGPITKESQPTIEEDLTRV
YKAISAQADKQDISNSTRVEFEKLYSDLMKFLESSKEIDPSQTTQLAGMD
VEDALDKDSTKEVASLPNKSTISKTVSSTIPRETIPFLHLRKKTPAGDWK
YDRQLSSLFLDGLEKAAFNGVTFKDKYVLITGAGKGSIGAEVLQGLLQGG
AKVVVTTSRFSKQVTDYYQSIYAKYGAKGSTLIVVPFNQGSKQDVEALIE
FIYDTEKNGGLGWDLDAIIPFAAIPEQGIELEHIDSKSEFAHRIMLTNIL
RMMGCVKKQKSARGIETRPAQVILPMSPNHGTFGGDGMYSESKLSLETLF
NRWHSESWANQLTVCGAIIGWTRGTGLMSANNIIAEGIEKMGVRTFSQKE
MAFNLLGLLTPEVVELCQKSPVMADLNGGLQFVPELKEFTAKLRKELVET
SEVRKAVSIETALEHKVVNGNSADAAYAQVEIQPRANIQLDFPELKPYKQ
VKQIAPAELEGLLDLERVIVVTGFAEVGPWGSARTRWEMEAFGEFSLEGC
VEMAWIMGFISYHNGNLKGRPYTGWVDSKTKEPVDDKDVKAKYETSILEH
SGIRLIEPELENGYNPEKKEMIQEVIVEEDLEPFEASKETAEQFKHQHGD
KVDIFEIPETGEYSVKLLKGATLYIPKALRFDRLVAGQIPTGWNAKTYGI
SDDIISQVDPITLFVLVSVVEAFIASGITDPYEMYKYVHVSEVGNCSGSG
MGGVSALRGMFKDRFKDEPVQNDILQESFINTMSAWVNMLLISSSGPIKT
PVGACATSVESVDIGVETILSGKARICIVGGYDDFQEEGSFEFGNMKATS
NTLEEFEHGRIPAEMSRPATTTRNGFMEAQGAGIQIIMQADLALKMGVPI
YGIVAMAATATDKIGRSVPAPGKGILTTAREHHSSVKYASPNLNMKYRKR
QLVTREAQIKDWVENELEALKLEAEEIPSEDQNEFLLERTREIHNEAESQ
LRAAQQQWGNDFYKRDPRIAPLRGALATYGLTIDDLGVASFHGTSTKAND
KNESATINEMMKHLGRSEGNPVIGVFQKFLTGHPKGAAGAWMMNGALQIL
NSGIIPGNRNADNVDKILEQFEYVLYPSKILKTDGVRAVSITSFGFGQKG
GQAIVVHPDYLYGAITEDRYNEYVAKVSAREKSAYKFFHNGMIYNKLFVS
KEHAPYTDELEEDVYLDPLARVSKDKKSGSLTFNSKNIQSKDSYINANTI
ETAKMIENMTKEKVSNGGVGVDVELITSINVENDTFIERNFTPQEIEYCS
AQPSVQSSFAGTWSAKEAVFKSLGVKSLGGGAALKDIEIVRVNKNAPAVE
LHGNAKKAAEEAGVTDVKVSISHDDLQAVAVAVSTKKGS

An additional exemplary enzyme having fatty acid synthase activity is FAS2 from Saccharomyces cerevisiae 288c, which is provided by the Accession No. P19097-1 and set forth as SEQ ID NO: 11.

(SEQ ID NO: 11)
MKPEVEQELAHILLTELLAYQFASPVRWIETQDVFLKDFNTERVVEIGPS
PTLAGMAQRTLKNKYESYDAALSLHREILCYSKDAKEIYYTPDPSELAAK
EEPAKEEAPAPTPAASAPAPAAAAPAPVAAAAPAAAAAEIADEPVKASLL
LHVLVAHKLKKSLDSIPMSKTIKDLVGGKSTVQNEILGDLGKEFGTTPEK
PEETPLEELAETFQDTFSGALGKQSSSLLSRLISSKMPGGFTITVARKYL
QTRWGLPSGRQDGVLLVALSNEPAARLGSEADAKAFLDSMAQKYASIVGV
DLSSAASASGAAGAGAAAGAAMIDAGALEEITKDHKVLARQQLQVLARYL
KMDLDNGERKFLKEKDTVAELQAQLDYLNAELGEFFVNGVATSFSRKKAR
TFDSSWNWAKQSLLSLYFEIIHGVLKNVDREVVSEAINIMNRSNDALIKF
MEYHISNTDETKGENYQLVKTLGEQLIENCKQVLDVDPVYKDVAKPTGPK
TAIDKNGNITYSEEPREKVRKLSQYVQEMALGGPITKESQPTIEEDLTRV
YKAISAQADKQDISSSTRVEFEKLYSDLMKFLESSKEIDPSQTTQLAGMD
VEDALDKDSTKEVASLPNKSTISKTVSSTIPRETIPFLHLRKKTPAGDWK
YDRQLSSLFLDGLEKAAFNGVTFKDKYVLITGAGKGSIGAEVLQGLLQGG
AKVVVTTSRFSKQVTDYYQSIYAKYGAKGSTLIVVPFNQGSKQDVEALIE
FIYDTEKNGGLGWDLDAIIPFAAIPEQGIELEHIDSKSEFAHRIMLINIL
RMMGCVKKQKSARGIETRPAQVILPMSPNHGTFGGDGMYSESKLSLETLF
NRWHSESWANQLTVCGAIIGWTRGIGLMSANNIIAEGIEKMGVRIFSQKE
MAFNLLGLLTPEVVELCQKSPVMADLNGGLQFVPELKEFTAKLRKELVET
SEVRKAVSIETALEHKVVNGNSADAAYAQVEIQPRANIQLDFPELKPYKQ
VKQIAPAELEGLLDLERVIVVTGFAEVGPWGSARTRWEMEAFGEFSLEGC
VEMAWIMGFISYHNGNLKGRPYTGWVDSKTKEPVDDKDVKAKYETSILEH
SGIRLIEPELENGYNPEKKEMIQEVIVEEDLEPFEASKETAEQFKHQHGD
KVDIFEIPETGEYSVKLLKGATLYIPKALRFDRLVAGQIPTGWNAKTYGI
SDDIISQVDPITLFVLVSVVEAFIASGITDPYEMYKYVHVSEVGNCSGSG
MGGVSALRGMFKDRFKDEPVQNDILQESFINTMSAWVNMLLISSSGPIKT
PVGACATSVESVDIGVETILSGKARICIVGGYDDFQEEGSFEFGNMKATS
NTLEEFEHGRIPAEMSRPATTTRNGFMEAQGAGIQIIMQADLALKMGVPI
YGIVAMAATATDKIGRSVPAPGKGILTTAREHHSSVKYASPNLNMKYRKR
QLVTREAQIKDWVENELEALKLEAEEIPSEDQNEFLLERTREIHNEAESQ
LRAAQQQWGNDFYKRDPRIAPLRGALATYGLTIDDLGVASFHGISTKAND
KNESATINEMMKHLGRSEGNPVIGVFQKFLTGHPKGAAGAWMMNGALQIL
NSGIIPGNRNADNVDKILEQFEYVLYPSKILKIDGVRAVSITSFGFGQKG
GQAIVVHPDYLYGAITEDRYNEYVAKVSAREKSAYKFFHNGMIYNKLFVS
KEHAPYTDELEEDVYLDPLARVSKDKKSGSLTFNSKNIQSKDSYINANTI
ETAKMIENMTKEKVSNGGVGVDVELITSINVENDTFIERNFTPQEIEYCS
AQPSVQSSFAGTWSAKEAVFKSLGVKSLGGGAALKDIEIVRVNKNAPAVE
LHGNAKKAAEEAGVTDVKVSISHDDLQAVAVAVSTKK

In some embodiments, the fatty acid synthase is a homolog of FAS2 from S. cerevisiae (SEQ ID NO: 5). In some embodiments, the enzyme having fatty acid synthase activity may be modified (e.g., mutated) to modulate activity of the enzymes.

Amino acids of the fatty acid synthase may be modified (e.g., substituted) to produce a FAS2 variant. For example, as described herein, the amino acid glycine at position 1250, referred to as glycine 1250 (G1250), of SEQ ID NO: 5, may be mutated to produce a FAS2 enzyme having a desired activity, such as increased production of ethyl-hexanoate and/or decreased production of hexanoic acid, during fermentation. In some embodiments, the amino acid corresponding to glycine 1250 of SEQ ID NO: 5 is substituted with an amino acid that is not a glycine residue (e.g., any other amino acid).

In some embodiments, the amino acid corresponding to glycine at position 1250 (G1250) of SEQ ID NO: 5 is substituted with an amino acid selected from alanine (A), arginine (R), lysine (K), aspartic acid (D), glutamic acid (E), serine (S), threonine (T), asparagine (N), glutamine (G), cysteine (C), histidine (H), proline (P), valine (V), isoleucine (I), leucine (L), methionine (M), phenylalanine (F), tyrosine (Y), or tryptophan (W).

In some embodiments, the amino acid corresponding to glycine at position 1250 (G1250) of SEQ ID NO: 5 is substituted with a nonpolar amino acid (e.g., alanine (A), valine (V), leucine (L), isoleucine (I), methionine (M), tryptophan (W), phenylalanine (F), proline (P)). In some embodiments, the amino acid corresponding to glycine at position 1250 (G1250) of SEQ ID NO: 5 is substituted with a polar amino acid (e.g., serine (S), threonine (T), cysteine (C), tyrosine (Y), asparagine (N), glutamine (G)). In some embodiments, the amino acid corresponding to glycine at position 1250 (G1250) of SEQ ID NO: 5 is substituted with a serine (S) residue (G1250S), provided by SEQ ID NO: 6. The substituted amino acid is denoted in boldface and underline below.

Amino Acid Sequence of Variant FAS2 from Saccharomyces cerevisiae—G1250S Mutation

(SEQ ID NO: 6)
MKPEVEQELAHILLTELLAYQFASPVRWIETQDVFLKDFNTERVVEIGPS
PTLAGMAQRTLKNKYESYDAALSLHREILCYSKDAKEIYYTPDPSELAAK
EEPAKEEAPAPTPAASAPAPAAAAPAPVAAAAPAAAAAEIADEPVKASLL
LHVLVAHKLKKSLDSIPMSKTIKDLVGGKSTVQNEILGDLGKEFGTTPEK
SEETPLEELAETFQDTFSGALGKQSSSLLSRLISSKMPGGFTITVARKYL
QTRWGLPSGRQDGVLLVALSNEPAARLGSEADAKAFLGSMAQKYASIVGV
DLSSAASASGAAGAGAAAGAAMIDAGALEEITKDHKVLARQQLQVLARYL
KMDLDNGERKFLKEKDTVAELQAQLDYLNAELGEFFVNGVATSFSRKKAR
TFDSSWNWAKQSLLSLYFEIIHGVLKNVDREVVSEAINIMNRSNDALIKF
MEYHISNTDETKGENYQLVKTLGEQLIENCKQVLDVDPVYKDVAKPTGPK
TAIDKNGNITYSEEPREKVRKLSQYVQEMALGGPITKESQPTIEEDLTRV
YKAISAQADKQDISNSTRVEFEKLYSDLMKFLESSKEIDPSQTTQLAGMD
VEDALDKDSTKEVASLPNKSTISKTVSSTIPRETIPFLHLRKKTPAGDWK
YDRQLSSLFLDGLEKAAFNGVTFKDKYVLITGAGKGSIGAEVLQGLLQGG
AKVVVTTSRFSKQVTDYYQSIYAKYGAKGSTLIVVPFNQGSKQDVEALIE
FIYDTEKNGGLGWDLDAIIPFAAIPEQGIELEHIDSKSEFAHRIMLINIL
RMMGCVKKQKSARGIETRPAQVILPMSPNHGTFGGDGMYSESKLSLETLF
NRWHSESWANQLTVCGAIIGWIRGIGLMSANNIIAEGIEKMGVRIFSQKE
MAFNLLGLLTPEVVELCQKSPVMADLNGGLQFVPELKEFTAKLRKELVET
SEVRKAVSIETALEHKVVNGNSADAAYAQVEIQPRANIQLDFPELKPYKQ
VKQIAPAELEGLLDLERVIVVTGFAEVGPWGSARTRWEMEAFGEFSLEGC
VEMAWIMGFISYHNGNLKGRPYTGWVDSKTKEPVDDKDVKAKYETSILEH
SGIRLIEPELENGYNPEKKEMIQEVIVEEDLEPFEASKETAEQFKHQHGD
KVDIFEIPETGEYSVKLLKGATLYIPKALRFDRLVAGQIPTGWNAKTYGI
SDDIISQVDPITLFVLVSVVEAFIASGITDPYEMYKYVHVSEVGNCSGSS
MGGVSALRGMFKDRFKDEPVQNDILQESFINTMSAWVNMLLISSSGPIKT
PVGACATSVESVDIGVETILSGKARICIVGGYDDFQEEGSFEFGNMKATS
NTLEEFEHGRIPAEMSRPATTTRNGFMEAQGAGIQIIMQADLALKMGVPI
YGIVAMAATATDKIGRSVPAPGKGILTTAREHHSSVKYASPNLNMKYRKR
QLVTREAQIKDWVENELEALKLEAEEIPSEDQNEFLLERTREIHNEAESQ
LRAAQQQWGNDFYKRDPRIAPLRGALATYGLTIDDLGVASFHGTSTKAND
KNESATINEMMKHLGRSEGNPVIGVFQKFLTGHPKGAAGAWMMNGALQIL
NSGIIPGNRNADNVDKILEQFEYVLYPSKTLKTDGVRAVSITSFGFGQKG
GQAIVVHPDYLYGAITEDRYNEYVAKVSAREKSAYKFFHNGMIYNKLFVS
KEHAPYTDELEEDVYLDPLARVSKDKKSGSLTFNSKNIQSKDSYINANTI
ETAKMIENMTKEKVSNGGVGVDVELITSINVENDTFIERNFTPQEIEYCS
AQPSVQSSFAGTWSAKEAVFKSLGVKSLGGGAALKDIEIVRVNKNAPAVE
LHGNAKKAAEEAGVTDVKVSISHDDLQAVAVAVSTKKGS

In some embodiments, the enzyme with fatty acid synthase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 5 or 6. In some embodiments, the enzyme with fatty acid synthase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 5 or 6 and contains a substitution mutation at the amino acid corresponding to glycine at position 1250 (G1250) of SEQ ID NO: 5. In some embodiments, the enzyme with fatty synthase activity comprises a substitution mutation of the amino acid corresponding to glycine at position 1250 (G1250) of SEQ ID NO: 5 with an amino acid that is not a glycine residue (e.g., any other amino acid). In some embodiments, the enzyme with fatty acid synthase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 5 and the amino acid corresponding to glycine at position 1250 (G1250) of SEQ ID NO: 5 is substituted with an amino acid selected from histidine (H), arginine (R), lysine (K), aspartic acid (D), glutamic acid (E), serine (S), threonine (T), asparagine (N), glutamine (G), cysteine (C), alanine (A), proline (P), valine (V), isoleucine (I), leucine (L), methionine (M), phenylalanine (F), tyrosine (Y), or tryptophan (W). In some embodiments, the enzyme with fatty acid synthase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 6.

In some embodiments, the enzyme with fatty acid synthase activity comprises an amino acid sequence as set forth in SEQ ID NO: 5. In some embodiments, the enzyme with fatty acid synthase activity consists of the amino acid sequence as set forth in SEQ ID NO: 5. In some embodiments, the enzyme with fatty acid synthase activity comprises an amino acid sequence as set forth in SEQ ID NO: 6. In some embodiments, the enzyme with fatty acid synthase activity consists of the amino acid sequence as set forth in SEQ ID NO: 6.

In some embodiments, the gene encoding the enzyme with fatty acid synthase activity comprises a nucleic acid sequence which encodes an enzyme comprising an amino acid sequence with at least 80% (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9%) sequence identity to the sequence as set forth in SEQ ID NO: 5 or 6. In some embodiments, the gene encoding the enzyme with fatty acid synthase activity comprises a nucleic acid sequence which encodes an enzyme comprising an amino acid sequence as set forth in SEQ ID NO: 5 or 6. In some embodiments, the gene encoding the enzyme with fatty acid synthase activity comprises a nucleic acid sequence which encodes an enzyme consisting of an amino acid sequence as set forth in SEQ ID NO: 5 or 6.

Identification of additional enzymes having fatty acid synthase activity or predicted to have fatty acid synthase activity may be performed, for example based on similarity or homology with one or more domains of an fatty acid synthase, such as the fatty acid synthase provided by SEQ ID NO: 5 or 6. In some embodiments, an enzyme for use in the modified cells and methods described herein may be identified based on similarity or homology with an active domain, such as a catalytic domain, such as a catalytic domain associated with fatty acid synthase activity. In some embodiments, an enzyme for use in the modified cells and methods described herein may have a relatively high level of sequence identity with a reference fatty acid synthase, e.g., a wild-type fatty acid synthase, such as SEQ ID NO: 5, in the region of the catalytic domain but a relatively low level of sequence identity to the reference fatty acid synthase based on analysis of a larger portion of the enzyme or across the full length of the enzyme. In some embodiments, the enzyme for use in the modified cells and methods described herein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9% sequence identity in the region of the catalytic domain of the enzyme relative to a reference fatty acid synthase (e.g., SEQ ID NO: 5).

In some embodiments, the enzyme for use in the modified cells and methods described herein has a relatively high level of sequence identity in the region of the catalytic domain of the enzyme relative to a reference fatty acid synthase (e.g., SEQ ID NO: 5 or 6) and a relatively low level of sequence identity to the reference fatty acid synthase based on analysis of a larger portion of the enzyme or across the full length of the enzyme. In some embodiments, the enzyme for use in the modified cells and methods described herein has at least 10%, at least 15%, at least 20%, at least 25%, at least 30% at least 35%, at least 40%, at least 45%, at least 50%, at least 55%, at least 60%, at least 65%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9% sequence identity based on a portion of the enzyme or across the full length of the enzyme relative to a reference fatty acid synthase (e.g., SEQ ID NO: 5 or 6).

This information can also be used to identify positions, e.g., corresponding positions, in other enzymes having or predicted to have fatty acid synthase activity. As will be evident to one of ordinary skill in the art, an amino acid substitution at a position identified in one fatty acid synthase enzyme can also be made in the corresponding amino acid position of another fatty acid synthase enzyme. In such instances, one of the fatty acid synthase enzymes may be used as a reference enzyme. For example, as described herein, amino acid substitutions at position G1250 of FAS2 from Saccharomyces cerevisiae (SEQ ID NO: 5) have been shown to result in engineered cells that increase production of ethyl-hexanoate. Similar amino acid substitutions can be made at the corresponding position of other enzymes having fatty acid synthase activity using FAS2 as a reference (e.g., SEQ ID NO: 5). For example, amino acid substitutions can be made at the corresponding position of a fatty acid synthase from another yeast species, another fungal species, another microorganism, or another eukaryote, as described herein, using FAS2 as a reference (e.g., SEQ ID NO: 5).

The fatty acid synthase variants described herein contain an amino acid substitution of one or more positions corresponding to a reference fatty acid synthase. In some embodiments, the fatty acid synthase variant contains an amino acid substitution at 1, 2, 3, 4, or more positions corresponding to a reference fatty acid synthase. In some embodiments, the fatty acid synthase is not a naturally occurring fatty acid synthase e.g., is genetically modified. In some embodiments, the fatty acid synthase does not have the amino acid sequence provided by SEQ ID NO: 5.

### Hexanoyl-CoA Synthetase (HCS) Enzymes

The genetically modified cells described herein contain, in some embodiments, a gene encoding an enzyme with hexanoyl-CoA synthetase (HCS) activity. In some embodiments, the gene is a heterologous gene. Hexanoyl-CoA synthetase (HCS) enzymes are acyl-activating enzymes (AAEs) that catalyze the formation of hexanoyl-CoA from the substrates hexanoic acid and free coenzyme A (CoA). Without wishing to be bound to any particular theory, expression of a hexanoyl-CoA synthetase during fermentation may reduce the final yield of hexanoic acid in a fermented product or beverage. Hexanoyl-CoA is a substrate of the enzymatic the reaction that forms ethyl-hexanoate, expression of a hexanoyl-CoA synthetase during fermentation may further increase the final yield of ethyl-hexanoate in a fermented product or beverage. Genetically modified cells expressing a hexanoyl-CoA synthetase enzyme may produce fermented products or beverages with higher levels of desired ethyl-hexanoate and lower concentrations of undesired hexanoic acid, compared to cells that do not express a hexanoyl-CoA synthetase.

In some embodiments, the hexanoyl-CoA synthetase gene is from a plant. In some embodiments, the hexanoyl-CoA synthetase gene is from a Cannabis species. In some embodiments, the hexanoyl-CoA synthetase gene is from Cannabis sativa.

An exemplary HCS enzyme is CsAAE1 from Cannabis sativa, which is provided by the Accession No. H9A1V3-1 and amino acid sequence set forth as SEQ ID NO: 7.

(SEQ ID NO: 7)
MGKNYKSLDSVVASDFIALGITSEVAETLHGRLAEIVCNYGAATPQTWIN
IANHILSPDLPFSLHQMLFYGCYKDFGPAPPAWIPDPEKVKSINLGALLE
KRGKEFLGVKYKDPISSFSHFQEFSVRNPEVYWRTVLMDEMKISFSKDPE
CILRRDDINNPGGSEWLPGGYLNSAKNCLNVNSNKKLNDTMIVWRDEGND
DLPLNKLTLDQLRKRVWLVGYALEEMGLEKGCAIAIDMPMHVDAVVIYLA
IVLAGYVVVSIADSFSAPEISTRLRLSKAKAIFTQDHIIRGKKRIPLYSR
VVEAKSPMAIVIPCSGSNIGAELRDGDISWDYFLERAKEFKNCEFTAREQ
PVDAYTNILFSSGTTGEPKAIPWTQATPLKAAADGWSHLDIRKGDVIVWP
TNLGWMMGPWLVYASLLNGASIALYNGSPLVSGFAKFVQDAKVTMLGVVP
SIVRSWKSTNCVSGYDWSTIRCFSSSGEASNVDEYLWLMGRANYKPVIEM
CGGTEIGGAFSAGSFLQAQSLSSFSSQCMGCTLYILDKNGYPMPKNKPGI
GELALGPVMFGASKILLNGNHHDVYFKGMPTLNGEVLRRHGDIFELTSNG
YYHAHGRADDTMNIGGIKISSIEIERVCNEVDDRVFETTAIGVPPLGGGP
EQLVIFFVLKDSNDTTIDLNQLRLSFNLGLQKKLNPLFKVTRVVPLSSLP
RTATNKIMRRVLRQQFSHFE.

In some embodiments, the heterologous gene encodes an enzyme with hexanoyl-CoA synthetase activity. In some embodiments, the heterologous gene encodes an enzyme with hexanoyl-CoA synthetase activity such that the enzyme reduces the levels of hexanoic acid in a fermented product or beverage. In some embodiments, the heterologous gene encodes an enzyme with hexanoyl-CoA synthetase activity such that the enzyme increases the levels of ethyl-hexanoate in a fermented product or beverage.

In some embodiments, the enzyme with hexanoyl-CoA synthetase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 7.

As described herein, when a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5% ,at least 98%, at least 98.5%, at least 99%, at least 99.5%, at least 99.6%, at least 99.7%, at least 99.8%, or at least 99.9% identity) and all increments thereof (e.g., tenths of a percent (i.e., 0.1%), hundredths of a percent (i.e., 0.01%), etc.).

In some embodiments, the enzyme with hexanoyl-CoA synthetase activity comprises an amino acid sequence as set forth in SEQ ID NO: 7. In some embodiments, the enzyme with hexanoyl-CoA synthetase activity consists of the amino acid sequence as set forth in SEQ ID NO: 7.

In some embodiments, the gene encoding the enzyme with hexanoyl-CoA synthetase activity comprises a nucleic acid sequence which encodes an enzyme comprising an amino acid sequence with at least 80% (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9%) sequence identity to the sequence as set forth in SEQ ID NO: 7. In some embodiments, the gene encoding the enzyme with hexanoyl-CoA synthetase activity comprises a nucleic acid sequence which encodes an enzyme comprising an amino acid sequence as set forth in SEQ ID NO: 7. In some embodiments, the gene encoding the enzyme with hexanoyl-CoA synthetase activity comprises a nucleic acid sequence which encodes an enzyme consisting of an amino acid sequence as set forth in SEQ ID NO: 7.

Identification of additional enzymes having hexanoyl-CoA synthetase activity or predicted to have hexanoyl-CoA synthetase activity may be performed, for example based on similarity or homology with one or more domains of an hexanoyl-CoA synthetase, such as the hexanoyl-CoA synthetase provided by SEQ ID NO: 7. In some embodiments, an enzyme for use in the modified cells and methods described herein may be identified based on similarity or homology with an active domain, such as a catalytic domain, such as a catalytic domain associated with hexanoyl-CoA synthetase activity. In some embodiments, an enzyme for use in the modified cells and methods described herein may have a relatively high level of sequence identity with a reference hexanoyl-CoA synthetase, e.g., a wild-type hexanoyl-CoA synthetase, such as SEQ ID NO: 7, in the region of the catalytic domain but a relatively low level of sequence identity to the reference hexanoyl-CoA synthetase based on analysis of a larger portion of the enzyme or across the full length of the enzyme. In some embodiments, the enzyme for use in the modified cells and methods described herein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9% sequence identity in the region of the catalytic domain of the enzyme relative to a reference hexanoyl-CoA synthetase (e.g., SEQ ID NO: 7).

In some embodiments, the enzyme for use in the modified cells and methods described herein has a relatively high level of sequence identity in the region of the catalytic domain of the enzyme relative to a reference hexanoyl-CoA synthetase (e.g., SEQ ID NO: 7) and a relatively low level of sequence identity to the reference hexanoyl-CoA synthetase based on analysis of a larger portion of the enzyme or across the full length of the enzyme. In some embodiments, the enzyme for use in the modified cells and methods described herein has at least 10%, at least 15%, at least 20%, at least 25%, at least 30% at least 35%, at least 40%, at least 45%, at least 50%, at least 55%, at least 60%, at least 65%, at least 88%, at least 89%, at least 90%, at least 91%, at least 92%, at least 93%, at least 94%, at least 95%, at least at least 96%, at least 96.5%, at least 97%, at least 97.5%, at least 98%, at least 98.5%, at least 99%, at least 99.5%, at least 99.6%, at least 99.7%, at least 99.8%, or at least 99.9% sequence identity based on a portion of the enzyme or across the full length of the enzyme relative to a reference hexanoyl-CoA synthetase (e.g., SEQ ID NO: 7).

### General Methods of Enzyme Modification

As will also be evident to one or ordinary skill in the art, the amino acid position number of a selected residue in an alcohol-O-acyltransferase, fatty acid synthase, and/or hexanoyl-CoA synthetase may have a different amino acid position number in another alcohol-O-acyltransferase, fatty acid synthase, and/or hexanoyl-CoA synthetase enzyme (e.g., a reference enzyme). Generally, one may identify corresponding positions in other alcohol-O-acyltransferase, fatty acid synthase, and/or hexanoyl-CoA synthetase enzymes using methods known in the art, for example by aligning the amino acid sequences of two or more enzymes. Software programs and algorithms for aligning amino acid (or nucleotide) sequences are known in the art and readily available, e.g., Clustal Omega (Sievers et al. 2011).

The alcohol-O-acyltransferase, fatty acid synthase, and/or hexanoyl-CoA synthetase variants described herein may further contain one or more additional modifications, for example to specifically alter a feature of the polypeptide unrelated to its desired physiological activity. Alternatively or in addition, the alcohol-O-acyltransferase, fatty acid synthase, and/or hexanoyl-CoA synthetase enzymes described herein may contain or more mutations to modulate expression and/or activity of the enzyme in the cell.

Mutations of a nucleic acid which encodes an alcohol-O-acyltransferase, fatty acid synthase, and/or hexanoyl-CoA synthetase preferably preserve the amino acid reading frame of the coding sequence, and preferably do not create regions in the nucleic acid which are likely to hybridize to form secondary structures, such a hairpins or loops, which can be deleterious to expression of the enzyme.

Mutations can be made by selecting an amino acid substitution, or by random mutagenesis of a selected site in a nucleic acid which encodes the polypeptide. As described herein, variant polypeptides can be expressed and tested for one or more activities to determine which mutation provides a variant polypeptide with the desired properties. Further mutations can be made to variants (or to non-variant polypeptides) which are silent as to the amino acid sequence of the polypeptide, but which provide preferred codons for translation in a particular host (referred to as codon optimization). The preferred codons for translation of a nucleic acid in, e.g., S. cerevisiae, are well known to those of ordinary skill in the art. Still other mutations can be made to the noncoding sequences of a gene or cDNA clone to enhance expression of the polypeptide. The activity of an alcohol-O-acyltransferase, fatty acid synthase, and/or hexanoyl-CoA synthetase (enzyme) variant can be tested by cloning the gene encoding the enzyme variant into an expression vector, introducing the vector into an appropriate host cell, expressing the enzyme variant, and testing for a functional capability of the enzyme, as disclosed herein.

The alcohol-O-acyltransferase, fatty acid synthase, and/or hexanoyl-CoA synthetase variants described herein may contain an amino acid substitution of one or more positions corresponding to a reference alcohol-O-acyltransferase, fatty acid synthase, and/or hexanoyl-CoA synthetase. In some embodiments, the alcohol-O-acyltransferase, fatty acid synthase, and/or hexanoyl-CoA synthetase variant contains an amino acid substitution at 1, 2, 3, 4, 5, or more positions corresponding to a reference alcohol-O-acyltransferase, fatty acid synthase, and/or hexanoyl-CoA synthetase. In some embodiments, the alcohol-O-acyltransferase, fatty acid synthase, and/or hexanoyl-CoA synthetase is not a naturally occurring alcohol-O-acyltransferase, fatty acid synthase, and/or hexanoyl-CoA synthetase, e.g., is genetically modified.

In some embodiments, the alcohol-O-acyltransferase, fatty acid synthase, and/or hexanoyl-CoA synthetase variant may also contain one or more amino acid substitutions that do not substantially affect the activity and/or structure of the alcohol-O-acyltransferase, fatty acid synthase, and/or hexanoyl-CoA synthetase enzyme. The skilled artisan will also realize that conservative amino acid substitutions may be made in the enzyme to provide functionally equivalent variants of the foregoing polypeptides, i.e., the variants retain the functional capabilities of the polypeptides. As used herein, a “conservative amino acid substitution” refers to an amino acid substitution which does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Exemplary functionally equivalent variants of polypeptides include conservative amino acid substitutions in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

As one of ordinary skill in the art would be aware, homologous genes encoding an enzyme having alcohol-O-acyltransferase could be obtained from other species and could be identified by homology searches, for example through a protein BLAST search, available at the National Center for Biotechnology Information (NCBI) internet site (ncbi.nlm.nih.gov). By aligning the amino acid sequence of an enzyme with one or more reference enzymes and/or by comparing the secondary or tertiary structure of a similar or homologous enzyme with one or more reference eta lyase, one can determine corresponding amino acid residues in similar or homologous enzymes and can determine amino acid residues for mutation in the similar or homologous enzyme.

Genes associated with the disclosure can be obtained (e.g., by PCR amplification) from DNA from any source of DNA which contains the given gene. In some embodiments, genes associated with the invention are synthetic, e.g., produced by chemical synthesis in vitro. Any means of obtaining a gene encoding the enzymes described herein are compatible with the modified cells and methods described herein.

The disclosure provided herein involves recombinant expression of genes encoding an enzyme having alcohol-O-acyltransferase, fatty acid synthase, and/or hexanoyl-CoA synthetase activity, functional modifications and variants of the foregoing, as well as uses relating thereto. Homologs and alleles of the nucleic acids associated with the invention can be identified by conventional techniques. Also encompassed by the invention are nucleic acids that hybridize under stringent conditions to the nucleic acids described herein. The term “stringent conditions” as used herein refers to parameters with which the art is familiar. Nucleic acid hybridization parameters may be found in references which compile such methods, e.g., Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York.

There are other conditions, reagents, and so forth which can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of nucleic acids of the invention (e.g., by using lower stringency conditions). The skilled artisan also is familiar with the methodology for screening cells and libraries for expression of such molecules which then are routinely isolated, followed by isolation of the pertinent nucleic acid molecule and sequencing.

The invention also includes degenerate nucleic acids which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code. The invention also embraces codon optimization to suit optimal codon usage of a host cell.

The invention also provides modified nucleic acid molecules which include additions, substitutions and deletions of one or more nucleotides. In preferred embodiments, these modified nucleic acid molecules and/or the polypeptides they encode retain at least one activity or function of the unmodified nucleic acid molecule and/or the polypeptides, such as enzymatic activity. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules which encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules which encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

In some embodiments, one or more of the genes associated with the invention is expressed in a recombinant expression vector. As used herein, a “vector” may be any of a number of nucleic acids into which a desired sequence or sequences may be inserted by restriction and ligation for transport between different genetic environments or for expression in a host cell. Vectors are typically composed of DNA although RNA vectors are also available. Vectors include, but are not limited to: plasmids, fosmids, phagemids, virus genomes and artificial chromosomes.

A cloning vector is one which is able to replicate autonomously or integrated in the genome in a host cell. In the case of plasmids, replication of the desired sequence may occur many times as the plasmid increases in copy number within the host cell such as a host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined or operably linked when they are covalently linked in such a way as to place the expression or transcription of the coding sequence under the influence or control of the regulatory sequences. If it is desired that the coding sequences be translated into a functional protein, two DNA sequences are said to be operably joined or operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the enzymes of the present disclosure is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene (e.g., an enzyme having alcohol-O-acyltransferase, fatty acid synthase, or hexanoyl-CoA synthetase activity). A variety of conditional promoters also can be used, such as promoters controlled by the presence or absence of a molecule.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Fourth Edition, Cold Spring Harbor Laboratory Press, 2012. Cells are genetically engineered by the introduction into the cells of heterologous DNA (RNA). That heterologous DNA (RNA) is placed under operable control of transcriptional elements to permit the expression of the heterologous DNA in the host cell. As one of ordinary skill in the art would appreciate, any of the enzymes described herein can also be expressed in other yeast cells, including yeast strains used for producing wine, mead, sake, cider, etc.

A nucleic acid molecule that encodes the enzyme of the present disclosure can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome.

The incorporation of genes can be accomplished either by incorporation of the new nucleic acid into the genome of the yeast cell, or by transient or stable maintenance of the new nucleic acid as an episomal element. In eukaryotic cells, a permanent, inheritable genetic change is generally achieved by introduction of the DNA into the genome of the cell.

The heterologous gene may also include various transcriptional elements required for expression of the encoded gene product (e.g., enzyme having alcohol-O-acyltransferase, fatty acid synthase, and/or hexanoyl-CoA synthetase). For example, in some embodiments, the gene may include a promoter. In some embodiments, the promoter may be operably joined to the gene. In some embodiments, the cell is an inducible promoter. In some embodiments, the promoter is active during a particular stage of a fermentation process. For example, in some embodiments, peak expression from the promoter is during an early stage of the fermentation process, e.g., before >50% of the fermentable sugars have been consumed. In some embodiments, peak expression from the promoter is during a late stage of the fermentation process e.g., after 50% of the fermentable sugars have been consumed.

Conditions in the medium change during the course of the fermentation process, for example the availability of nutrients and oxygen tend to decrease over time during fermentation as sugar source and oxygen become depleted. Additionally, the presence of other factors, such as products produced by metabolism of the cells, increase. In some embodiments, the promoter is regulated by one or more conditions in the fermentation process, such as presence or absence of one or more factors. In some embodiments, the promoter is regulated by hypoxic conditions. Examples of promoters of hypoxia activated genes are known in the art. See, e.g., Zitomer et al. Kidney Int. (1997) 51(2): 507-13; Gonzalez Siso et al. Biotechnol. Letters (2012) 34: 2161-2173.

In some embodiments, the promoter is a constitutive promoter. Examples of constitutive promoters for use in yeast cells are known in the art and evident to one of ordinary skill in the art. In some embodiments, the promoter is a yeast promoter, e.g., a native promoter from the yeast cell in which the heterologous gene or the exogenous gene is expressed.

In some embodiments, the promoter is the HEM13 promoter (pHEM13), SPG1 promoter (pSPG1), PRB1 promoter (pPRB1), QCR10 (pQCR10), PGK1 promoter (pPGK1), OLE1 promoter (pOLE1), ERG25 promoter (pERG25), or the HHF2 promoter (pHHF2).

An exemplary HEM13 promoter is pHEM13 from S. cerevisiae, which is provided by the nucleotide sequence set forth as SEQ ID NO: 8.

(SEQ ID NO: 8)
TAATGTAGAAGGTTGAGAACAACCGGATCTTGCGGTCATTTTTCTTTTCG
AGGAAAGTGCAAGTCTGCCACTTTCCAGAAGGCATAGCCTTGCCCTTTTG
TTGATATTTCTCCCCACCGTAATTGTTGCATTCGCGATCTTTTCAACAAT
ACATTTTATCATCAAGCCCGCAAATCCTCTGGAGTTTGTCCTCTCGTTCA
CTGTTGGGAAAAACAATACGCCTAATTCGTGATTAAGATTCTTCAAACCA
TTTCCTGCGGAGTTTTTACTGTGTGTTGAACGGTTCACAGCGTAAAAAAA
AGTTACTATAGGCACGGTATTTTAATTTCAATTGTTTAGAAAGTGCCTTC
ACACCATTAGCCCCTGGGATTACCGTCATAGGCACTTTCTGCTGAGCTCC
TGCGAGATTTCTGCGCTGAAAGAGTAAAAGAAATCTTTCACAGCGGCTCC
GCGGGCCCTTCTACTTTTAAACGAGTCGCAGGAACAGAAGCCAAATTTCA
AAGAACGCTACGCTTTCGCCTTTTCTGGTTCTCCCACCAATAACGCTCCA
GCTTGAACAAAGCATAAGACTGCAACCAAAGCGCTGACGGACGATCCGAA
GATAAAGCTTGCTTTGCCCATTGTTCTCGTTTCGAAAGGCTATATAAGGA
CACGGATTTTCCtTTTTTTTTTCCACCTATTGTCTTTCTTTGTTAAGCTT
TTATTCTCCGGGTTTTtTTTTTTTGAGCATATCAAAAGCTTTCTTTTCGC
AAATCAAACATAGCAAACCGAACTCTTCGAACACAATTAAATACACATAA
A.

An exemplary SPG1 promoter is pSPG1 from S. cerevisiae, which is provided by the nucleotide sequence set forth as SEQ ID NO: 9.

(SEQ ID NO: 9)
ATGAAGTTCACTTCACATCCAATGAGAAAAACAAAATCCGCAGGGCTATC
ACCCAGAACATCCTCCACTTCATCTTCTTCAGGACAGAGAAAAGCGCATC
ACCACCACCATCACCACAACCACGTTTCAAGGACGAAAACTACCGAAAGC
ACCAAATCAGGCAACAGCAAAAAGGACAGTTCCTCATCCTCAACAAACGA
CCATCAATTTAAAAGGTCTGAAAAGAAGAAAAAAAGTAAATTTGGCTCGA
TCTTCAAAAAAGTTTTCGGATGAACCGGATTAATACAAGTAAAATCAGCA
AAGATATAGAAGACAAAATAAGCGTGAAAACAATCATAAACCACTCACAA
CGGGGGTTTTCAGCTGTTACTCCTCCATACATACATTTTGATAAAGATAT
AATGTTATATTTCTTTTCGTAATTTTGTTTTACTTCGGTTTGCTCTATAG
ATTTCATCAGCCGCACCGAAAAGGGAGATCAATAAGGTACCCTTTAAAAG
GGATAAGAAGCCTAACATCACCCCAATAAATGGAGTAATGGCCAGCATTG
GATGAAGAGAAGAATTACGGGATACTGGGATAACACTGTTAAAAATGCTT
CGCGACGTGAGGGTCTTATATAAATTGAACTGCCAAATCTCTTTCACATT
ATCCAGGATAGTTTGGAATGTGTGTTACTGAAAGATCAGAATCAATAAAT
ACAATCAATACAAATATTTAGCGCATAAAATTCAAACAAAGTTTACTGAA.

An exemplary PRB1 promoter is pPRB1 from S. cerevisiae, which is provided by the nucleotide sequence set forth as SEQ ID NO: 10.

(SEQ ID NO: 10)
CGAGAAACAGGGGGGGAGAAAAGGGGAAAAGAGAAGGAAAGAAAGACTCA
TCTATCGCAGATAAGACAATCAACCCTCATGGCGCCTCCAACCACCATCC
GCACTAGGGACCAAGCGCTCGCACCGTTAGCAACGCTTGACTCACAAACC
AACTGCCGGCTGAAAGAGCTTGTGCAATGGGAGTGCCAATTCAAAGGAGC
CGAATACGTCTGTTCGCCTTTTAAGAGGCTTTTTGAACACTGCATTGCAC
CCGACAAATCAGCCACTAACTACGAGGTCACGGATACATATACCAATAGT
TAAAAAATTACATATACTCTATATAGCACAGTAGTGTGATAAATAAAAAA
TTTTGCCAAGACTTTTTTAAACTGCACCCGACAGATCAGGTCTGTGCCTA
CTATGCACTTATGCCCGGGGTCCCGGGAGGAGAAAAAACGAGGGCTGGGA
AATGTCCGTGGACTTAAAACGCTCCGGGTTAGCAGAGTAGCAGGGCTTTC
GGCTTTGGAAATTTAGGTGACTTGTTGAAAAAGCAAAATTTGGGCTCAGT
AATGCCACaGCAGTGGCTTATCACGCCAGGACTGCGGGAGTGGCGGGGGC
AAACACACCCGCGATAAAGAGCGCGATGAATATAAAAGGGGGCCAATGTT
ACGTCCCGTTATATTGGAGTTCTTCCCATACAAACTTAAGAGTCCAATTA
GCTTCATCGCCAATAAAAAAACAAACTAAACCTAATTCTAACAAGCAAAG.

An exemplary QCR10 promoter is pQCR10 from S. cerevisiae, which is provided by the nucleotide sequence set forth as SEQ ID NO: 22.

(SEQ ID NO: 22)
GAGAGCTGGCCAAAAAGAGGGCCGAAGACGGCGTTGAATTTCATTCAAAA
CTATTTAGAAGGGCAGAGCCAGGTGAGGATTTAGATTATTATATTTACAA
GCACATCCCTGAAGGGACCGACAAGCATGAAGAACAGATCAGGAGCATTT
TGGAAACTGCCCCGATTTTACCAGGACAGGCATTCACTGAAAAATTTTCT
ATTCCGGCTTATAAAAAGCATGGAATCCAAAAGAATTAGGCTTCTCATTC
TATTTTAATTATACTAGTACGATTTCTCACTCTGTAATTTAATATCAGTG
TAATATGCACCTAGTTATGGGTAGTTTTTGCTAACGTTACGAGCCGCGAA
ACTGTCCTCAATCTTCACCACTACCTCTAATGACTGAAGAATGCTATGCG
ATATAACGCTGCCGCACTTTGAATATATACTTATATTTACATAGTTTTCA
AGTGCGTATTACTATTGCAAAGTAGTATTTTGTCACGTGATTTTGATCCA
ATTAAAACTAAATATGGTTCAACCCGTTGTTTCCGCATCAAAAAACCATA
CCATTTATCAAGGGGACGGGATATATCACATAACAGTTTGAATGCATAAT
TTGTTATAGATATCTTCTGGAATAATCTTCACAGCAAAAGCGCAAGTCGA
ATAATATATCGATAAATACAATCCATAAGACTTAAAACTAACCTCA.

### Genetically Modified Yeast Cells

Aspects of the present disclosure relates to genetically modified yeast cells (modified cells) and use of such modified cells in methods of producing a fermented product (e.g., a fermented beverage) and methods of producing ethanol. The genetically modified yeast cells described herein are genetically modified with a heterologous gene encoding an enzyme with alcohol-O-acyltransferase activity, an exogenous gene encoding an enzyme with fatty acid synthase activity, and/or a heterologous gene encoding an enzyme with hexanoyl-CoA synthetase activity.

The terms “genetically modified cell,” “genetically modified yeast cell,” and “modified cell,” as may be used interchangeably herein, to refer to a eukaryotic cell (e.g., a yeast cell, which has been, or may be presently, modified by the introduction of a heterologous gene. The terms (e.g., modified cell) include the progeny of the original cell which has been genetically modified by the introduction of a heterologous gene. It shall be understood by the skilled artisan that the progeny of a single cell may not necessarily be completely identical in morphology or in genomic or total nucleic acid complement as the original parent, due to mutation (i.e., natural, accidental, or deliberate alteration of the nucleic acids of the modified cell).

Yeast cells for use in the methods described herein are preferably capable of fermenting a sugar source (e.g., a fermentable sugar) and producing ethanol (ethyl alcohol) and carbon dioxide. In some embodiments, the yeast cell is of the genus Saccharomyces. The Saccharomyces genus includes nearly 500 distinct of species, many of which are used in food production. One example species is Saccharomyces cerevisiae (S. cerevisiae), which is commonly referred to as “brewer's yeast” or “baker's yeast,” and is used in the production of wine, bread, beer, among other products. Other members of the Saccharomyces genus include, without limitation, the wild yeast Saccharomyces paradoxus, which is a close relative to S. cerevisiae; Saccharomyces bayanus, Saccharomyces pastorianus, Saccharomyces carlsbergensis, Saccharomyces uvarum, Saccharomyces cerevisiae var boulardii, Saccharomyces eubayanus. In some embodiments, the yeast is Saccharomyces cerevisiae (S. cerevisiae).

Saccharomyces species may be haploid (i.e., having a single set of chromosomes), diploid (i.e., having a paired set of chromosomes), or polyploid (i.e., carrying or containing more than two homologous sets of chromosomes). Saccharomyces species used, for example for beer brewing, are typically classified into two groups: ale strains (e.g., S. cerevisiae), which are top fermenting, and lager strains (e.g., S. pastorianus, S. carlsbergensis, S. uvarum), which are bottom fermenting. These characterizations reflect their separation characteristics in open square fermentors, as well as often other characteristics such as preferred fermentation temperatures and alcohol concentrations achieved.

Although beer brewing and wine producing has traditionally focused on use of S. cerevisiae strains, other yeast genera have been appreciated in production of fermented beverages. In some embodiments, the yeast cell belongs to a non-Saccharomyces genus. See, e.g., Crauwels et al. Brewing Science (2015) 68: 110-121; Esteves et al. Microorganisms (2019) 7(11): 478. In some embodiments, the yeast cell is of the genus Kloeckera, Candida, Starmerella, Hanseniaspora, Kluyveromyces/Lachance, Metschnikowia, Saccharomycodes, Zygosaccharomyce, Dekkera (also referred to as Brettanomyces), Wickerhamomyces, or Torulaspora. Examples of non-Saccharomyces yeast include, without limitation, Hanseniaspora uvarum, Hanseniaspora guillermondii, Hanseniaspora vinae, Metschnikowia pulcherrima, Kluyveromyces/Lachancea thermotolerans, Starmerella bacillaris (previously referred to as Candida stellata/Candida zemplinina), Saccharomycodes ludwigii, Zygosaccharomyces rouxii, Dekkera bruxellensis, Dekkera anomala, Brettanomyces custersianus, Brettanomyces naardenensis, Brettanomyces nanus, Wickerhamomyces anomalus, and Torulaspora delbrueckii.

In some embodiments, the methods described herein involve use of more than one genetically modified yeast. For example, in some embodiments, the methods may involve use of more than one genetically modified yeast belonging to the genus Saccharomyces. In some embodiments, the methods may involve use of more than one genetically modified yeast belonging to a non-Saccharomyces genus. In some embodiments, the methods may involve use of more than one genetically modified yeast belonging to the genus Saccharomyces and one genetically modified yeast belonging to a non-Saccharomyces genus. Alternatively or in addition, the any of the methods described herein may involve use of one or more genetically modified yeast and one or more non-genetically modified (wildtype) yeast.

In some embodiments, the yeast is a hybrid strain. As will be evident to one of ordinary skill in the art, the term “hybrid strain” of yeast refers to a yeast strain that has resulted from the crossing of two different yeast strains, for example, to achieve one or more desired characteristics. For example, a hybrid strain may result from the crossing of two different yeast strains belonging to the same genus or the same species. In some embodiments, a hybrid strain results from the crossing of a Saccharomyces cerevisiae strain and a Saccharomyces eubayanus strain. See, e.g., Krogerus et al. Microbial Cell Factories (2017) 16:66.

In some embodiments, the yeast strain is a wild yeast strain, such as a yeast strain that is isolated from a natural source and subsequently propagated. Alternatively, in some embodiments, the yeast strain is a domesticated yeast strain. Domesticated yeast strains have been subjected to human selection and breeding to have desired characteristics.

In some embodiments, the genetically modified yeast cells may be used in symbiotic matrices with bacterial strains and used for the production of fermented beverages, such as kombucha, kefir, and ginger beers. Saccharomyces fragilis, for example, is part of kefir culture and is grown on the lactose contained in whey.

Methods of genetically modifying yeast cells are known in the art. In some embodiments, the yeast cell is diploid and one copy of a heterologous gene encoding an enzyme with alcohol-O-acyltransferase activity as described herein is introduced into the yeast genome.

In some embodiments, the yeast cell is diploid and one copy of a heterologous gene encoding an enzyme with alcohol-O-acyltransferase activity as described herein is introduced into both copies of the yeast genome. In some embodiments, the copies of the heterologous gene are identical. In some embodiments, the copies of the heterologous gene are not identical, but the genes encode an identical enzyme having alcohol-O-acyltransferase activity. In some embodiments, the copies of the heterologous gene are not identical, and the genes encode enzymes having alcohol-O-acyltransferase activity that are different (e.g., mutants, variants, fragments thereof).

In some embodiments, the yeast cell is diploid and one copy of a gene encoding an enzyme with fatty acid synthase activity as described herein is introduced into both copies of the yeast genome. In some embodiments, the copies of the gene encoding an enzyme with fatty acid synthase activity are identical. In some embodiments, the copies of the gene encoding an enzyme with fatty acid synthase activity are not identical, but the genes encode an identical enzyme having fatty acid synthase activity. In some embodiments, the copies of the gene encoding an enzyme with fatty acid synthase activity are not identical, and the genes encode enzymes having fatty acid synthase activity that are different (e.g., mutants, variants, fragments thereof). In some embodiments, the cell contains a gene encoding an enzyme with fatty acid synthase activity, referred to as an endogenous gene, and also contains a second gene encoding an enzyme with fatty acid synthase activity, which may be the same or different enzyme with fatty acid synthase activity as that encoded by the endogenous gene.

In some embodiments, the yeast cell is diploid and one copy of a heterologous gene encoding an enzyme with hexanoyl-CoA synthetase activity as described herein is introduced into both copies of the yeast genome. In some embodiments, the copies of the heterologous gene are identical. In some embodiments, the copies of the heterologous gene are not identical, but the genes encode an identical enzyme having hexanoyl-CoA synthetase activity. In some embodiments, the copies of the heterologous gene are not identical, and the genes encode enzymes having hexanoyl-CoA synthetase activity that are different (e.g., mutants, variants, fragments thereof).

In some embodiments, the yeast cell is tetraploid. Tetraploid yeast cells are cells which maintain four complete sets of chromosomes (i.e., a complete set of chromosomes in four copies). In some embodiments, the yeast cell is tetraploid and a copy of a heterologous gene encoding an enzyme with alcohol-O-acyltransferase activity as described herein is introduced into at least one copy of the genome. In some embodiments, the yeast cell is tetraploid and a copy of a heterologous gene encoding an enzyme with alcohol-O-acyltransferase activity as described herein is introduced into more than one copy of the genome. In some embodiments, the yeast cell is tetraploid and a copy of a heterologous gene encoding an enzyme with alcohol-O-acyltransferase activity as described herein is introduced all four copies of the genome. In some embodiments, the copies of the heterologous gene are identical. In some embodiments, the copies of the heterologous gene are not identical, but the genes encode an identical enzyme having alcohol-O-acyltransferase activity. In some embodiments, the copies of the heterologous gene are not identical, and the genes encode enzymes having alcohol-O-acyltransferase activity that are different (e.g., mutants, variants, fragments thereof).

In some embodiments, the yeast cell is tetraploid and a copy of a gene encoding an enzyme with fatty acid synthase activity as described herein is introduced into at least one copy of the genome. In some embodiments, the yeast cell is tetraploid and a copy of a gene encoding an enzyme with fatty acid synthase activity as described herein is introduced into more than one copy of the genome. In some embodiments, the yeast cell is tetraploid and a copy of a gene encoding an enzyme with fatty acid synthase activity as described herein is introduced all four copies of the genome. In some embodiments, the copies of the gene encoding an enzyme with fatty acid synthase activity are identical. In some embodiments, the copies of the gene encoding an enzyme with fatty acid synthase activity are not identical, but the genes encode an identical enzyme having fatty acid synthase activity. In some embodiments, the copies of the gene encoding an enzyme with fatty acid synthase activity are not identical, and the genes encode enzymes having fatty acid synthase activity that are different (e.g., mutants, variants, fragments thereof). In some embodiments, the cell contains a gene encoding an enzyme with fatty acid synthase activity, referred to as an endogenous gene, and also contains one or more additional copies of a gene encoding an enzyme with fatty acid synthase activity, which may be the same or different enzyme with fatty acid synthase activity as that encoded by the endogenous gene.

In some embodiments, the yeast cell is tetraploid and a copy of a heterologous gene encoding an enzyme with hexanoyl-CoA synthetase activity as described herein is introduced into at least one copy of the genome. In some embodiments, the yeast cell is tetraploid and a copy of a heterologous gene encoding an enzyme with hexanoyl-CoA synthetase activity as described herein is introduced into more than one copy of the genome. In some embodiments, the yeast cell is tetraploid and a copy of a heterologous gene encoding an enzyme with hexanoyl-CoA synthetase activity as described herein is introduced all four copies of the genome. In some embodiments, the copies of the heterologous gene are identical. In some embodiments, the copies of the heterologous gene are not identical, but the genes encode an identical enzyme having hexanoyl-CoA synthetase activity. In some embodiments, the copies of the heterologous gene are not identical, and the genes encode enzymes having hexanoyl-CoA synthetase activity that are different (e.g., mutants, variants, fragments thereof).

In some embodiments, the growth rate of the modified cell is not substantially impaired relative to a wild-type yeast cell that does not comprise the first heterologous gene and second exogenous gene. Methods of measuring and comparing the growth rates of two cells will be known to one of ordinary skill in the art. Non-limiting examples of growth rates that can be measured and compared between two types of cells are replication rate, budding rate, colony-forming units (CFUs) produced per unit of time, and amount of fermentable sugar reduced in a medium per unit of time. The growth rate of a modified cell is “not substantially impaired” relative to a wild-type cell if the growth rate, as measured, is at least 70%, at least 75%, at least 80%, at least 85%, at least 90%, at least 95%, at least 99%, or at least 100% of the growth rate of the wild-type cell.

Strains of yeast cells that may be used with the methods described herein will be known to one of ordinary skill in the art and include yeast strains used for brewing desired fermented beverages as well as commercially available yeast strains. Examples of common beer strains include, without limitation, American ale strains, Belgian ale strains, British ale strains, Belgian lambic/sour ale strains, Barleywine/Imperial Stout strains, India Pale Ale strains, Brown Ale strains, Kolsch and Altbier strains, Stout and Porter strains, and Wheat beer strains.

Non-limiting examples of yeast strains for use with the genetically modified cells and methods described herein include Wyeast American Ale 1056, Wyeast American Ale II 1272, Wyeast Denny's Favorite 50 1450, Wyeast Northwest Ale 1332, Wyeast Ringwood Ale 1187, Siebel Inst. American Ale BRY 96, White Labs American Ale Yeast Blend WLP060, White Labs California Ale V WLP051, White Labs California Ale WLP001, White Labs Old Sonoma Ale WLP076, White Labs Pacific Ale WLP041, White Labs East Coast Ale WLP008, White Labs East Midlands Ale WLP039, White Labs San Diego Super Yeast WLP090, White Labs San Francisco Lager WLP810, White Labs Neutral Grain WLP078, Lallemand American West Coast Ale BRY-97, Lallemand CBC-1 (Cask and Bottle Conditioning), Brewferm Top, Coopers Pure Brewers' Yeast, Fermentis US-05, Real Brewers Yeast Lucky #7, Muntons Premium Gold, Muntons Standard Yeast, East Coast Yeast Northeast Ale ECY29, East Coast Yeast Old Newark Ale ECY10, East Coast Yeast Old Newark Beer ECY12, Fermentis Safale US-05, Fermentis Safbrew T-58, Real Brewers Yeast The One, Mangrove Jack US West Coast Yeast, Mangrove Jack Workhorse Beer Yeast, Lallemand Abbaye Belgian Ale, White Labs Abbey IV WLP540, White Labs American Farmhouse Blend WLP670, White Labs Antwerp Ale WLP515, East Coast Yeast Belgian Abbaye ECY09, White Labs Belgian Ale WLP550, Mangrove Jack Belgian Ale Yeast, Wyeast Belgian Dark Ale 3822-PC, Wyeast Belgian Saison 3724, White Labs Belgian Saison I WLP565, White Labs Belgian Saison II WLP566, White Labs Belgian Saison III WLP585, Wyeast Belgian Schelde Ale 3655-PC, Wyeast Belgian Stout 1581-PC, White Labs Belgian Style Ale Yeast Blend WLP575, White Labs Belgian Style Saison Ale Blend WLP568, East Coast Yeast Belgian White ECY11, Lallemand Belle Saison, Wyeast Biere de Garde 3725-PC, White Labs Brettanomyces Bruxellensis Trois Vrai WLP648, Brewferm Top, Wyeast Canadian/Belgian Ale 3864-PC, Lallemand CBC-1 (Cask and Bottle Conditioning), Wyeast Farmhouse Ale 3726-PC, East Coast Yeast Farmhouse Brett ECY03, Wyeast Flanders Golden Ale 3739-PC, White Labs Flemish Ale Blend WLP665, White Labs French Ale WLP072, Wyeast French Saison 3711, Wyeast Leuven Pale Ale 3538-PC, Fermentis Safbrew T-58, East Coast Yeast Saison Brasserie Blend ECY08, East Coast Yeast Saison Single-Strain ECY14, Real Brewers Yeast The Monk, Siebel Inst. Trappist Ale BRY 204, East Coast Yeast Trappist Ale ECY13, White Labs Trappist Ale WLP500, Wyeast Trappist Blend 3789-PC, Wyeast British Ale 1098, Wyeast British Ale II 1335, Wyeast British Cask Ale 1026-PC, Wyeast English Special Bitter 1768-PC, Wyeast Irish Ale 1084, Wyeast London Ale 1028, Wyeast London Ale III 1318, Wyeast London ESB Ale 1968, Wyeast Ringwood Ale 1187, Wyeast Thames Valley Ale 1275, Wyeast Thames Valley Ale II 1882-PC, Wyeast West Yorkshire Ale 1469, Wyeast Whitbread Ale 1099, Mangrove Jack British Ale Yeast, Mangrove Jack Burton Union Yeast, Mangrove Jack Workhorse Beer Yeast, East Coast Yeast British Mild Ale ECY18, East Coast Yeast Northeast Ale ECY29, East Coast Yeast Burton Union ECY17, East Coast Yeast Old Newark Ale ECY10, White Labs Bedford British Ale WLP006, White Labs British Ale WLP005, White Labs Burton Ale WLP023, White Labs East Midlands Ale WLP039, White Labs English Ale Blend WLP085, White Labs English Ale WLP002, White Labs Essex Ale Yeast WLP022, White Labs Irish Ale WLP004, White Labs London Ale WLP013, White Labs Manchester Ale WLP038, White Labs Old Sonoma Ale WLP076, White Labs San Diego Super Yeast WLP090, White Labs Whitbread Ale WLP017, White Labs North Yorkshire Ale WLP037, Coopers Pure Brewers' Yeast, Siebel Inst. English Ale BRY 264, Muntons Premium Gold, Muntons Standard Yeast, Lallemand Nottingham, Fermentis Safale S-04, Fermentis Safbrew T-58, Lallemand Windsor (British Ale), Real Brewers Yeast Ye Olde English, Brewferm Top, White Labs American Whiskey WLP065, White Labs Dry English Ale WLP007, White Labs Edinburgh Ale WLP028, Fermentis Safbrew S-33, Wyeast Scottish Ale 1728, East Coast Yeast Scottish Heavy ECY07, White Labs Super High Gravity WLP099, White Labs Whitbread Ale WLP017, Wyeast Belgian Lambic Blend 3278, Wyeast Belgian Schelde Ale 3655-PC, Wyeast Berliner-Weisse Blend 3191-PC, Wyeast Brettanomyces Bruxellensis 5112, Wyeast Brettanomyces Lambicus 5526, Wyeast Lactobacillus 5335, Wyeast Pediococcus Cerevisiae 5733, Wyeast Roeselare Ale Blend 3763, Wyeast Trappist Blend 3789-Pc, White Labs Belgian Sour Mix W1p655, White Labs Berliner Weisse Blend W1p630, White Labs Saccharomyces “Bruxellensis” Trois W1p644, White Labs Brettanomyces Bruxellensis W1p650, White Labs Brettanomyces Claussenii W1p645, White Labs

Brettanomyces Lambicus W1p653, White Labs Flemish Ale Blend W1p665, East Coast Yeast Berliner Blend Ecy06, East Coast Yeast Brett Anomala Ecy04, East Coast Yeast Brett Bruxelensis Ecy05, East Coast Yeast Brett Custersianus Ecy19, East Coast Yeast Brett Nanus Ecy16, Strain #2, East Coast Yeast BugCounty ECY20, East Coast Yeast BugFarm ECY01, East Coast Yeast Farmhouse Brett ECY03, East Coast Yeast Flemish Ale ECY02, East Coast Yeast Oud Brune ECY23, Wyeast American Ale 1056, Siebel Inst. American Ale BRY 96, White Labs American Ale Yeast Blend WLP060, White Labs Bourbon Yeast WLP070, White Labs California Ale V WLP051, White Labs California Ale WLP001, White Labs Dry English ale WLP007, White Labs East Coast Ale WLP008, White Labs Neutral Grain WLP078, White Labs Super High Gravity WLP099, White Labs Tennessee WLP050, Fermentis US-05, Real Brewers Yeast Lucky #7, Fermentis Safbrew S-33, East Coast Yeast Scottish Heavy ECY07, Lallemand Windsor (British Ale), Wyeast American Ale 1056, Wyeast American Ale II 1272, Wyeast British Ale 1098, Wyeast British Ale II 1335, Wyeast Denny's Favorite 50 1450, Wyeast London Ale 1028, Wyeast London Ale III 1318, Wyeast London ESB Ale 1968, Wyeast Northwest Ale 1332, Wyeast Ringwood Ale 1187, Siebel Inst. American Ale BRY 96, White Labs American Ale Yeast Blend WLP060, White Labs Bedford British Ale WLP006, White Labs British Ale WLP005, White Labs Burton Ale WLP023, White Labs California Ale V WLP051, White Labs California Ale WLP001, White Labs East Coast Ale WLP008, White Labs English Ale WLP002, White Labs London Ale WLP013, White Labs Essex Ale Yeast WLP022, White Labs Pacific Ale WLP041, White Labs San Diego Super Yeast WLP090, White Labs Whitbread Ale WLP017, Brewferm Top, Mangrove Jack Burton Union Yeast, Mangrove Jack US West Coast Yeast, Mangrove Jack Workhorse Beer Yeast, Coopers Pure Brewers' Yeast, Fermentis US-05, Fermentis Safale S-04, Fermentis Safbrew T-58, Real Brewers Yeast Lucky #7, Real Brewers Yeast The One, Muntons Premium Gold, Muntons Standard Yeast, East Coast Yeast Northeast Ale ECY29, Lallemand Nottingham, Lallemand Windsor (British Ale), Wyeast American Ale 1056, Wyeast American Ale II 1272, Wyeast British Ale 1098, Wyeast British Ale II 1335, Wyeast Thames Valley Ale 1275, Wyeast Thames Valley Ale II 1882-PC, Wyeast West Yorkshire Ale 1469, Wyeast Whitbread Ale 1099, Wyeast British Cask Ale 1026-PC, Wyeast English Special Bitter 1768-PC, Wyeast London Ale 1028, Wyeast London Ale III 1318, Wyeast London ESB Ale 1968, Wyeast Northwest Ale 1332, Wyeast Ringwood Ale 1187, White Labs American Ale Yeast Blend WLP060, White Labs British Ale WLP005, White Labs Bedford British Ale WLP006, White Labs British Ale WLP005, White Labs Burton Ale WLP023, White Labs California Ale V WLP051, White Labs California Ale WLP001, White Labs East Coast Ale WLP008, White Labs English Ale WLP002, White Labs Essex Ale Yeast WLP022, White Labs French Ale WLP072, White Labs London Ale WLP013, White Labs Pacific Ale WLP041, White Labs Whitbread Ale WLP017, Brewferm Top, East Coast Yeast British Mild Ale ECY18, Coopers Pure Brewers' Yeast, Muntons Premium Gold, Muntons Standard Yeast, Mangrove Jack Newcastle Dark Ale Yeast, Lallemand CBC-1 (Cask and Bottle Conditioning), Lallemand Nottingham, Lallemand Windsor (British Ale), Fermentis Safale S-04, Fermentis US-05, Siebel Inst. American Ale BRY 96, Wyeast American Wheat 1010, Wyeast German Ale 1007, Wyeast Kolsch 2565, Wyeast Kolsch II 2575-PC, White Labs Belgian Lager WLP815, White Labs Dusseldorf Alt WLP036, White Labs European Ale WLP011, White Labs German Ale/Kolsch WLP029, East Coast Yeast Kolschbier ECY21, Mangrove Jack Workhorse Beer Yeast, Siebel Inst. Alt Ale BRY 144, Wyeast American Ale 1056, Wyeast American Ale II 1272, Wyeast British Ale 1098, Wyeast British Ale II 1335, Wyeast Denny's Favorite 50 1450, Wyeast English Special Bitter 1768-PC, Wyeast Irish Ale 1084, Wyeast London Ale 1028, Wyeast London Ale III 1318, Wyeast London ESB Ale 1968, Wyeast Northwest Ale 1332, Wyeast Ringwood Ale 1187, Wyeast Thames Valley Ale 1275, Wyeast Thames Valley Ale II 1882-PC, Wyeast West Yorkshire Ale 1469, Wyeast Whitbread Ale 1099, White Labs American Ale Yeast Blend WLP060, White Labs Bedford British Ale WLP006, White Labs British Ale WLP005, White Labs Burton Ale WLP023, White Labs California Ale V WLP051, White Labs California Ale WLP001, White Labs East Coast Ale WLP008, White Labs East Midlands Ale WLP039, White Labs English Ale WLP002, White Labs Essex Ale Yeast WLP022, White Labs Irish Ale WLP004, White Labs London Ale WLP013, White Labs Old Sonoma Ale WLP076, White Labs Pacific Ale WLP041, White Labs Whitbread Ale WLP017, Coopers Pure Brewers' Yeast, Fermentis US-05, Muntons Premium Gold, Muntons Standard Yeast, Fermentis Safale S-04, Lallemand Nottingham, Lallemand Windsor (British Ale), Siebel Inst. American Ale BRY 96, White Labs American Hefeweizen Ale 320, White Labs Bavarian Weizen Ale 351, White Labs Belgian Wit Ale 400, White Labs Belgian Wit Ale II 410, White Labs Hefeweizen Ale 300, White Labs Hefeweizen IV Ale 380, Wyeast American Wheat 1010, Wyeast Bavarian Wheat 3638, Wyeast Bavarian Wheat Blend 3056, Wyeast Belgian Ardennes 3522, Wyeast Belgian Wheat 3942, Wyeast Belgian Witbier 3944, Wyeast Canadian/Belgian Ale 3864-PC, Wyeast Forbidden Fruit Yeast 3463, Wyeast German Wheat 3333, Wyeast Weihenstephan Weizen 3068, Siebel Institute Bavarian Weizen BRY 235, Fermentis Safbrew WB-06, Mangrove Jack Bavarian Wheat, Lallemand Munich (German Wheat Beer), Brewferm Blanche, Brewferm Lager, East Coast Yeast Belgian White ECY11. In some embodiments, the yeast is S. cerevisiae strain WLP001 California Ale (which may be referred to as “CA01”).

In some embodiments, the yeast strain for use with the genetically modified cells and methods described herein is a wine yeast strain. Examples of yeast strains for use with the genetically modified cells and methods described herein include, without limitation, Red Star Montrachet, EC-1118, Elegance, Red Star Côte des Blancs, Epernay II, Red Star Premier Cuvee, Red Star Pasteur Red, Red Star Pasteur Champagne, Fermentis BCS-103, and Fermentis VR44. In some embodiments, the yeast is S. cerevisiae strain Elegance. In some embodiments, the yeast is S. cerevisiae strain EC-1118 (also referred to as EC1118 or Lalvin EC 1118® (Lallemand Brewing).

In some embodiments, the modified cell is an S. cerevisiae cell that expresses FAS2 under control of a PRB1 promoter and MpAAT1-A169G,A170F under control of a PGK1 promoter. In some embodiments, the modified cell also comprises a deletion of EHT1 and EEB1.

In some embodiments, the modified cell is an S. cerevisiae cell that expresses FAS2 under the control of a PRB1 promoter and MpAAT1-A169G,A170F under the control of a PGK1 promoter.

In some embodiments, the modified cell is an S. cerevisiae that expresses FAS2-G1250S under control of a PRB1 promoter and MpAAT1-A169G,A170F under control of a PGK1 promoter. In some embodiments, the modified cell also comprises a deletion of EHT1.

In some embodiments, the modified cell is an S. cerevisiae cell that expresses FAS2-G1250S under control of a PRB1 promoter and MpAAT1-A169G,A170F under control of a PGK1 promoter. In some embodiments, the modified cell also comprises a deletion of EHT1 and EEB1.

In some embodiments, the modified cell is an S. cerevisiae cell that expresses FAS2-G1250S under control of a PRB1 promoter and MpAAT1-A169G,A170F under control of a PGK1 promoter. In some embodiments, the modified cell also comprises a deletion of EHT1 EEB1, and MGL2.

In some embodiments, the modified cell is an S. cerevisiae cell that expresses FAS2-G1250S under control of a PRB1 promoter, MpAAT1-A169G,A170F under control of a PGK1 promoter, and HCS under control of a PDC6 promoter. In some embodiments, the modified cell also comprises a deletion of EHT1 EEB1, and MGL2.

In some embodiments, the modified cell is an S. cerevisiae cell that expresses FAS2-G1250S under control of a PRB1 promoter and MaWES1 under control of a QCR10 promoter. In some embodiments, the modified cell is an S. cerevisiae cell that expresses FAS2-G1250S under control of a PRB1 promoter and MaWES1 under control of a HEM13 promoter. In some embodiments, the modified cell also comprises a deletion of EHT1 and EEB1.

### Methods

Aspects of the present disclosure relate to methods of producing a fermented product using any of the genetically modified yeast cells described herein. Also provided are methods of producing ethanol using any of the genetically modified yeast cells described herein.

The process of fermentation exploits a natural process of using microorganisms to convert carbohydrates into alcohol and carbon dioxide. It is a metabolic process that produces chemical changes in organic substrates through enzymatic action. In the context of food production, fermentation broadly refers to any process in which the activity of microorganisms brings about a desirable change to a food product or beverage. The conditions for fermentation and the carrying out of a fermentation is referred to herein as a “fermentation process.”

In some aspects, the disclosure relates to a method of producing a fermented product, such as a fermented beverage, involving contacting any of the modified cell described herein with a medium comprising at least one fermentable sugar during a first fermentation process, to produce a fermented product. A “medium” as used herein, refers to liquid conducive to fermentation, meaning a liquid which does not inhibit or prevent the fermentation process. In some embodiments, the medium is water. In some embodiments, the methods of producing a fermented product involve contacting purified enzymes (e.g., any of the alcohol-O-acyltransferase, fatty acid synthase, and/or hexanoyl-CoA synthetase enzymes described herein) with a medium comprising at least one fermentable sugar during a first fermentation process, to produce a fermented product.

As also used herein, the term “fermentable sugar” refers to a carbohydrate that may be converted into an alcohol and carbon dioxide by a microorganism, such as any of the cells described herein. In some embodiments, the fermentable sugar is converted into an alcohol and carbon dioxide by an enzyme, such as a recombinant enzyme or a cell that expresses the enzyme. Examples of fermentable sugars include, without limitation, glucose, fructose, lactose, sucrose, maltose, and maltotriose.

In some embodiments, the fermentable sugar is provided in a sugar source. The sugar source for use in the claimed methods may depend, for example, on the type of fermented product and the fermentable sugar. Examples of sugar sources include, without limitation, wort, grains/cereals, fruit juice (e.g., grape juice and apple juice/cider), honey, cane sugar, rice, and koji. Examples of fruits from which fruit juice can be obtained include, without limitation, grapes, apples, blueberries, blackberries, raspberries, currants, strawberries, cherries, pears, peaches, nectarines, oranges, pineapples, mangoes, and passionfruit.

As will be evident to one of ordinary skill in the art, in some instances, it may be necessary to process the sugar source in order to make available the fermentable sugar for fermentation. Using beer production as an example fermented beverage, grains (cereal, barley) are boiled or steeped in water, which hydrates the grain and activates the malt enzymes converting the starches to fermentable sugars, referred to as “mashing.” As used herein, the term “wort” refers to the liquid produced in the mashing process, which contains the fermentable sugars. The wort then is exposed to a fermenting organism (e.g., any of the cells described herein), which allows enzymes of the fermenting organism to convert the sugars in the wort to alcohol and carbon dioxide. In some embodiments, the wort is contacted with a recombinant enzyme (e.g., any of the enzymes described herein), which may optionally be purified or isolated from an organism that produces the enzyme, allowing the enzyme to convert the sugars in the wort to alcohol and carbon dioxide.

In some embodiments, the grains are malted, unmalted, or comprise a combination of malted and unmalted grains. Examples of grains for use in the methods described herein include, without limitation, barley, oats, maize, rice, rye, sorghum, wheat, karasumugi, and hatomugi.

In the example of producing sake, the sugar source is rice, which is incubated with koji mold (Aspergillus oryzae) converting the rice starch to fermentable sugar, producing koji. The koji then is exposed to a fermenting organism (e.g., any of the cells described herein), which allows enzymes of the fermenting organism to convert the sugars in the koji to alcohol and carbon dioxide. In some embodiments, the koji is contacted with a recombinant enzyme (e.g., any of the enzymes described herein), which may optionally be purified or isolated from an organism that produces the enzyme, allowing the enzyme to convert the sugars in the koji to alcohol and carbon dioxide.

In the example of producing wine, grapes are harvested, mashed (e.g., crushed) into a composition containing the skins, solids, juice, and seeds. The resulting composition is referred to as the “must.” The grape juice may be separated from the must and fermented, or the entirety of the must (i.e., with skins, seeds, solids) may be fermented. The grape juice or must then is exposed to a fermenting organism (e.g., any of the cells described herein), which allows enzymes of the fermenting organism to convert the sugars in the grape juice or must to alcohol and carbon dioxide. In some embodiments, the grape juice or must is contacted with a recombinant enzyme (e.g., any of the enzymes described herein), which may optionally be purified or isolated from an organism that produces the enzyme, allowing the enzyme to convert the sugars in the grape juice or must to alcohol and carbon dioxide.

In some embodiments, the methods described herein involve producing the medium, which may involve heating or steeping a sugar source, for example in water. In some embodiments, the water has a temperature of at least 50 degrees Celsius (50° C.) and incubated with a sugar source of a period of time. In some embodiments, the water has a temperature of at least 75° C. and incubated with a sugar source of a period of time. In some embodiments, the water has a temperature of at least 100° C. and incubated with a sugar source of a period of time. Preferably, the medium is cooled prior to addition of any of the cells described herein.

In some embodiments, the methods described herein further comprise adding at least one (e.g., 1, 2, 3, 4, 5, or more) hop variety for example to the medium, to a wort during a fermentation process. Hops are the flowers of the hops plant (Humulus lupulus) and are often used in fermentation to impart various flavors and aromas to the fermented product. Hops are considered to impart bitter flavoring in addition to floral, fruity, and/or citrus flavors and aromas and may be characterized based on the intended purpose. For example, bittering hops impart a level of bitterness to the fermented product due to the presence of alpha acids in the hop flowers, whereas aroma hops have lower lowers of alpha acids and contribute desirable aromas and flavor to the fermented product.

Whether one or more variety of hops is added to the medium and/or the wort and at stage during which the hops are added may be based on various factors, such as the intended purpose of the hops. For example, hops that are intended to impart a bitterness to the fermented product are typically added to during preparation of the wort, for example during boiling of the wort. In some embodiments, hops that are intended to impart a bitterness to the fermented product are added to the wort and boiled with the wort for a period of time, for example, for about 15-60 minutes. In contrast, hops that are intended to impart desired aromas to the fermented product are typically added later than hops used for bitterness. In some embodiments, hops that are intended to impart desired aromas to the fermented product are added to at the end of the boil or after the wort is boiled (i.e., “dry hopping”). In some embodiments, one or more varieties of hops may be added at multiple times (e.g., at least twice, at least three times, or more) during the methods.

In some embodiments, the hops are added in the form of either wet or dried hops and may optionally be boiled with the wort. In some embodiments, the hops are in the form of dried hop pellets. In some embodiments, at least one variety of hops is added to the medium. In some embodiments, the hops are wet (i.e., undried). In some embodiment, the hops are dried, and optionally may be further processed prior to use. In some embodiments, the hops are added to the wort prior to the fermentation process. In some embodiments, the hops are boiled in the wort. In some embodiments, the hops are boiled with the wort and then cooled with the wort.

Many varieties of hops are known in the art and may be used in the methods described herein. Examples of hop varieties include, without limitation, Ahtanum, Amarillo, Apollo, Cascade, Centennial, Chinook, Citra, Cluster, Columbus, Crystal/Chrystal, Eroica, Galena, Glacier, Greenburg, Horizon, Liberty, Millennium, Mosaic, Mount Hood, Mount Rainier, Newport, Nugget, Palisade, Santiam, Simcoe, Sterling, Summit, Tomahawk, Ultra, Vanguard, Warrior, Willamette, Zeus, Admiral, Brewer's Gold, Bullion, Challenger, First Gold, Fuggles, Goldings, Herald, Northdown, Northern Brewer, Phoenix, Pilot, Pioneer, Progress, Target, Whitbread Golding Variety (WGV), Hallertau, Hersbrucker, Saaz, Tettnang, Spalt, Feux-Coeur Francais, Galaxy, Green Bullet, Motueka, Nelson Sauvin, Pacific Gem, Pacific Jade, Pacifica, Pride of Ringwood, Riwaka, Southern Cross, Lublin, Magnum, Perle, Polnischer Lublin, Saphir, Satus, Select, Strisselspalt, Styrian Goldings, Tardif de Bourgogne, Tradition, Bravo, Calypso, Chelan, Comet, El Dorado, San Juan Ruby Red, Satus, Sonnet Golding, Super Galena, Tillicum, Bramling Cross, Pilgrim, Hallertauer Herkules, Hallertauer Magnum, Hallertauer Taurus, Merkur, Opal, Smaragd, Halleratau Aroma, Kohatu, Rakau, Stella, Sticklebract, Summer Saaz, Super Alpha, Super Pride, Topaz, Wai-iti, Bor, Junga, Marynka, Premiant, Sladek, Styrian Atlas, Styrian Aurora, Styrian Bobek, Styrian Celeia, Sybilla Sorachi Ace, Hallertauer Mittelfrueh, Hallertauer Tradition, Tettnanger, Tahoma, Triple Pearl, Yahima Gold, and Michigan Copper.

In some embodiments, the fermentation process of at least one sugar source comprising at least one fermentable sugar may be carried out for about 1 day to about 31 days. In some embodiments, the fermentation process is performed for about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days or longer. In some embodiments, the fermentation process of the one or more fermentable sugars may be performed at a temperature of about 4° C. to about 30° C. In some embodiments, the fermentation process of one or more fermentable sugars may be carried out at temperature of about 8° C. to about 14° C. or about 18° C. to about 24° C. In some embodiments, the fermentation process of one or more fermentable sugars may be performed at a temperature of about 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., or 30° C.

In some embodiments, fermentation results in the reduction of the amount of fermentable sugar present in a medium. In some embodiments, the reduction in the amount of fermentable sugar occurs within 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, or longer, from the start of fermentation. In some embodiments, the amount of fermentable sugar is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, at least 99.5%, at least 99.6%, at least 99.7%, at least 99.8%, at least 99.9%, or 100%. In some embodiments, the modified cell or cells ferment a comparable or greater amount of fermentable sugar, relative to the amount of fermentable sugar fermented by wild-type yeast cells in the same amount of time.

The methods described herein may involve at least one additional fermentation process. Such additional fermentation methods may be referred to as secondary fermentation processes (also referred to as “aging” or “maturing”). As will be understood by one of ordinary skill in the art, secondary fermentation typically involves transferring a fermented beverage to a second receptacle (e.g., glass carboy, barrel) where the fermented beverage is incubated for a period of time. In some embodiments, the secondary fermentation is performed for a period of time between 10 minutes and 12 months. In some embodiments, the secondary fermentation is performed for 10 minutes, 20 minutes, 40 minutes, 40 minutes, 50 minutes, 60 minutes (1 hour), 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days, 5 days, 6 days, 7 days, 8 days, 9 days, 10 days, 11 days, 12 days, 13 days, 14 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 3 months, 4 months, 5 months, 6 months, 7 months, 8 months, 9 months, 10 months, 11 months, 12 months, or longer. In some embodiments, the additional or secondary fermentation process of the one or more fermentable sugars may be performed at a temperature of about 4° C. to about 30° C. In some embodiments, the additional or secondary fermentation process of one or more fermentable sugars may be carried out at temperature of about 8° C. to about 14° C. or about 18° C. to about 24° C. In some embodiments, the additional or secondary fermentation process of one or more fermentable sugars may be performed at a temperature of about 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., or 30° C.

As will be evident to one of ordinary skill in the art, selection of a time period and temperature for an additional or secondary fermentation process will depend on factors such as the type of beer, the characteristics of the beer desired, and the yeast strain used in the methods.

In some embodiments, one or more additional flavor component may be added to the medium prior to or after the fermentation process. Examples include, hop oil, hop aromatics, hop extracts, hop bitters, and isomerized hops extract.

Products from the fermentation process may volatilize and dissipate during the fermentation process or from the fermented product. For example, ethyl-hexanoate produced during fermentation using the cells described herein may volatilize resulting in reduced levels of ethyl-hexanoate in the fermented product. In some embodiments, volatilized ethyl-hexanoate is captured and re-introduced after the fermentation process.

Various refinement, filtration, and aging processes may occur subsequent fermentation, after which the liquid is bottled (e.g., captured and sealed in a container for distribution, storage, or consumption). Any of the methods described herein may further involve distilling, pasteurizing and/or carbonating the fermented product. In some embodiments, the methods involve carbonating the fermented product. Methods of carbonating fermented beverages are known in the art and include, for example, force carbonating with a gas (e.g., carbon dioxide, nitrogen), naturally carbonating by adding a further sugar source to the fermented beverage to promote further fermentation and production of carbon dioxide (e.g., bottle conditioning).

### Fermented Products

Aspects of the present disclosure relate to fermented products produced by any of the methods disclosed herein. In some embodiments, the fermented product is a fermented beverage. Examples of fermented beverages include, without limitation, beer, wine, sake, mead, cider, cava, sparkling wine (champagne), kombucha, ginger beer, water kefir. In some embodiments, the beverage is beer. In some embodiments, the beverage is wine. In some embodiments, the beverage is sparkling wine. In some embodiments, the beverage is Champagne. In some embodiments, the beverage is sake. In some embodiments, the beverage is mead. In some embodiments, the beverage is cider. In some embodiments, the beverage is hard seltzer. In some embodiments, the beverage is a wine cooler.

In some embodiments, the fermented product is a fermented food product. Examples of fermented food products include, without limitation, cultured yogurt, tempeh, miso, kimchi, sauerkraut, fermented sausage, bread, soy sauce.

According to aspects of the invention, increased titers of ethyl-hexanoate are produced through the recombinant expression of genes associated with the invention, in yeast cells and use of the cells in the methods described herein. As used herein, an “increased titer” or “high titer” refers to a titer in the nanograms per liter (ng L−1) scale. The titer produced for a given product will be influenced by multiple factors including the choice of medium and conditions for fermentation.

In some embodiments, the titer of ethyl-hexanoate is at least 1 μg L⁻¹, for example at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1050, 1100, 1200, 1300, 1400, 1500, 1600, 1700, 1800, 1900, 2000, 2100, 2200, 2300, 2400, 2500, 2600, 2700, 2800, 2900, 3000 μg L⁻¹.

Aspects of the present disclosure relate to reducing the production of undesired products (e.g., byproducts, off-flavors), such as hexanoic acid, during fermentation of a product. In some embodiments, expression of the alcohol-O-acyltransferases, fatty acid synthases, and/or hexanoyl-CoA synthetases in the genetically modified cells described herein result in a reduction in the production of an undesired product by about 5%, 10%, 15%, 20%, 25%, 30%, 35%, 40%, 45%, 50%, 55%, 60%, 65%, 70%, 75%, 80%, 85%, 90%, 95% or more relative to production of the undesired product (e.g., hexanoic acid) by use of a wild-type yeast cell or a yeast cell that does not express the enzymes.

In some embodiments, the titer of hexanoic acid is less than 1000 mg L¹, for example less than 1000, 950, 900, 850, 800, 750, 700, 650, 600, 550, 500, 450, 400, 350, 300, 250, 200, 150, 100, 95, 90, 85, 80, 75, 70, 65, 60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1 mg L⁻¹ or less.

Methods of measuring titers/levels of ethyl-hexanoate and/or hexanoic acid will be evident to one of ordinary skill in the art. In some embodiments, the titers/levels of ethyl-hexanoate and/or hexanoic acid are measured using gas-chromatograph mass-spectrometry (GC/MS). In some embodiments, the titers/levels of ethyl-hexanoate and/or hexanoic acid are assessed using sensory panels, including for example human taste-testers.

In some embodiments, the fermented beverage contains an alcohol by volume (also referred to as “ABV,” “abv,” or “alc/vol”) between 0.1% and 30%. In some embodiments, the fermented beverage contains an alcohol by volume of about 0.1%, 0.2%, 0.3%, 0.4%, 0.6%, 0.07%, 0.8%, 0.9%, 1.0%, 1.1%, 1.2%, 1.3%, 1.4%, 1.5%, 1.6%, 1.7%, 1.8%, 1.9%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or higher. In some embodiments, the fermented beverage is non-alcoholic (e.g., has an alcohol by volume less than 0.5%).

### Kits

Aspects of the present disclosure also provides kits for use of the genetically modified yeast cells, for example to produce a fermented beverage, fermented product, or ethanol. In some embodiments, the kit contains a modified cell containing a heterologous gene encoding an enzyme with alcohol-O-acyltransferase (AAT) activity, an exogenous gene encoding an enzyme with fatty acid synthase (FAS2) activity, and/or a heterologous gene encoding an enzyme with hexanoyl-CoA (HCS) activity.

In some embodiments, the kit is for the production of a fermented beverage. In some embodiments, the kit is for the production of beer. In some embodiments, the kit is for the production of wine. In some embodiments, the kit is for the production of sake. In some embodiments, the kit is for the production of mead. In some embodiments, the kit is for the production of cider.

The kits may also comprise other components for use in any of the methods described herein, or for use of any of the cells as described herein. For example, in some embodiments, the kits may contain grains, water, wort, must, yeast, hops, juice, or other sugar source(s). In some embodiments, the kit may contain one or more fermentable sugars. In some embodiments, the kit may contain one or more additional agents, ingredients, or components.

Instructions for performing the methods described herein may also be included in the kits described herein.

The kits may be organized to indicate a single-use compositions containing any of the modified cells described herein. For example, the single use compositions (e.g., amount to be used) can be packaged compositions (e.g., modified cells) such as packeted (i.e., contained in a packet) powders, vials, ampoules, culture tube, tablets, caplets, capsules, or sachets containing liquids.

The compositions (e.g., modified cells) may be provided in dried, lyophilized, frozen, or liquid forms. In some embodiments, the modified cells are provided as colonies on an agar medium. In some embodiments, the modified cells are provided in the form of a starter culture that may be pitched directly into a medium. When reagents or components are provided as a dried form, reconstitution generally is by the addition of a solvent, such as a medium. The solvent may be provided in another packaging means and may be selected by one skilled in the art.

A number of packages or kits are known to those skilled in the art for dispensing a composition (e.g., modified cells). In certain embodiments, the package is a labeled blister package, dial dispenser package, tube, packet, drum, or bottle.

Any of the kits described herein may further comprise one or more vessel for performing the methods described herein, such as a carboy or barrel.

### GENERAL TECHNIQUES

The practice of the subject matter of the disclosure will employ, unless otherwise indicated, conventional techniques of molecular biology (including recombinant techniques), microbiology, cell biology, biochemistry and immunology, which are within the skill of the art. Such techniques are explained fully in the literature, such as, but without limiting, Molecular Cloning: A Laboratory Manual, J. Sambrook, et al., eds., Fourth Edition, Cold Spring Harbor Laboratory Press, Cold Spring Harbor, New York, 2012; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999).

### EQUIVALENTS AND SCOPE

It is to be understood that this disclosure is not limited to any or all of the particular embodiments described expressly herein, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this disclosure are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents (i.e., any lexicographical definition in the publications and patents cited that is not also expressly repeated in the disclosure should not be treated as such and should not be read as defining any terms appearing in the accompanying claims). If there is a conflict between any of the incorporated references and this disclosure, this disclosure shall control. In addition, any particular embodiment of this disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

In the claims articles such as “a,” “an,” and “the” may mean one or more than one unless indicated to the contrary or otherwise evident from the context. Wherever used herein, a pronoun in a gender (e.g., masculine, feminine, neuter, other, etc) the pronoun shall be construed as gender neutral (i.e., construed to refer to all genders equally) regardless of the implied gender unless the context clearly indicates or requires otherwise. Wherever used herein, words used in the singular include the plural, and words used in the plural includes the singular, unless the context clearly indicates or requires otherwise. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists (e.g., in Markush group format), each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included in such ranges unless otherwise specified. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the disclosure, as defined in the following claims.

### EXAMPLES

### Example 1

### Identification of an AAT for Improved Ethyl-Hexanoate Biosynthesis

To develop genetically modified cells that produce increased levels of ethyl-hexanoate during beer and wine fermentation, production of ethyl-hexanoate is balanced with maintaining hexanoic acid concentrations below the flavor detection threshold and growth/replication of the resulting genetically modified cells. First, candidate alcohol-O-acyltransferases (AAT) that may produce ethyl-hexanoate but had minimal ester hydrolase and acyl-CoA thioesterase activity were identified. Given that the AAT enzyme family is large and functionally diverse, it was hypothesized that a non-endogenous yeast AAT may display superior activity in this regard compared to endogenous yeast enzymes. A literature search identified a set of 11 candidate enzymes from fungal, bacterial, and plant origins that had previously been shown to, or were likely to have, ethyl-hexanoate biosynthesis activity. Genes encoding the candidate AAT enzymes were synthesized and transformed into a California Ale brewing yeast strain under transcriptional control of the strong glycolytic promoter, pPGK1. Transformed strains were grown semi-anaerobically in brewing wort media to simulate beer fermentation. After five days of fermentation, samples of each culture were run on a GC-MS to measure ethyl-hexanoate and hexanoic acid concentrations in the media. Data from this experiment revealed that expression of a variant AAT from Marinobacter aquaeolei (hereinafter referred to as “MaWES”) resulted in the highest concentration of ethyl-hexanoate and also the highest ratio of ethyl-hexanoate to hexanoic acid. Ethyl-hexanoate and hexanoic acid levels in fermentations with strains expressing MaWES were 5-fold higher and 2-folder higher, respectively, than in fermentations using strains overexpres sing an endogenous yeast AAT, EEB1.

The MaWES AAT enzyme was previously evaluated to exploit its activity for the production of biofuels. Two single amino acid mutations were found to alter the substrate specificity of the enzyme. For example, Barney et al. found that an A360I mutation increased the relative binding affinity of MaWES for C8-C10 alcohol substrates, while reducing affinity for C12-14 alcohol substrates. See, Barney et al. Appl. Environ. Microbiol. (2013) 79: 5734-5745. In addition, Petronikolou and Nair found that an A144F mutation increased binding affinity for hexanoyl-CoA, while reducing affinity for longer acyl-CoA substrates. See, Petronikolou et al. ACS Catal. (2018) 8: 6334-6344. However, production of ethyl-hexanoate or ester flavor molecules by either the wild-type or mutant MaWES enzymes were not evaluated.

Substitution mutations were introduced at positions A360 and A144 of MaWE (A360I,A144F) and the resulting strain was evaluated for ethyl-hexanoate biosynthesis compared to the wild-type enzyme. A Cal Ale yeast strain expressing the MaWES mutant enzyme (MaWES_{A360I,A144F}) under the control of the constitutive 3-phosphoglycerate kinase promoter pPGK1 was generated. This strain, referred to as BY719, was used to brew beer in 5-gallon fermentations.

Beer brewed with BY719 was analyzed by a sensory tasting panel, and the concentrations of ethyl-hexanoate and hexanoic acid were quantified by gas chromatography/mass spectroscopy (GC/MS) analysis. Tasting panel notes indicated that the beer did contain very mild pineapple flavors but that goaty and sweet off-flavors were also present. Consistent with these tasting notes, GC/MS analysis revealed that ethyl-hexanoate concentrations in the beer were 2-fold higher than in beer brewed with a control, non-engineered (wild-type) strain, but that hexanoic acid levels were 4-fold higher than in the control beer. Additionally, in contrast to the control strain, strains expressing the MaWES mutant enzyme (MaWES_{A360I,A144F}) did not fully metabolize all of the fermentable sugars present in the brewing wort. Such “incomplete fermentations” generally result from strain engineering efforts that produce off-target effects that negatively affect cellular energetics or increase production of growth-inhibitory metabolic byproducts. Incomplete fermentations often result in sweet, high calorie beers that are generally not commercially viable.

Based on these experimental fermentations, it was concluded that strong expression of the MaWES mutant enzyme (MaWES_{A360I,A144F}) resulted in more ethyl-hexanoate and a higher ratio of ethyl-hexanoate to hexanoic acid than S. cerevisiae WLP001 strains analogously engineered for EEB1 over-expression. Second, the concentration of ethyl-hexanoate in beers brewed by BY719 was likely too low to have a meaningful effect on beer flavor, and the hexanoic acid concentration was high enough to be above the human detection threshold, imparting undesirable goaty off-flavors. Third, expression of the MaWES mutant enzyme (MaWES_{A360I,A144F}) resulted in strain growth defects that inhibited BY719 from fully consuming the fermentable sugars present in the beer fermentation. These findings demonstrated that yeast expressing the MaWES mutant enzyme (MaWES_{A360I,A144F}) showed potential for improving pineapple flavors in fermented beverages but that further development was needed to 1) further increase production of ethyl-hexanoate, 2) reduce hexanoic acid production, and 3) eliminate strain growth defects.

### Improving Ethyl-Hexanoate Production by Combining MaWES Expression with Increased Biosynthesis of Hexanoyl-CoA

The BY719 strain was further engineered to increase the concentration of ethyl-hexanoate produced during fermentation. Because hexanoyl-CoA is a substrate in the reaction generating ethyl-hexanoate and may thus be a limiting compound, yeast strains were engineered to express a fatty acid synthase subunit alpha (FAS2) containing with a G1250S mutation, to increase production of hexanoyl-CoA. To this end, a G1250S mutation was introduced at the endogenous FAS2 locus in the yeast genome. The FAS2 G1250S strain was engineered to express the MaWES mutant enzyme (MaWES_{A360I,A144F}) driven by the delta-9 fatty acid desaturase promoter pOLE1, a medium strength promoter, resulting in the strain referred to as BY580.

BY580 was grown in small scale brewing fermentations, after which ethyl-hexanoate and hexanoic acid production, as well as sugar consumption, were measured. This strain produced more ethyl-hexanoate and more hexanoic acid as compared to BY719. However, similar to BY719, strain BY580 also grew poorly and did not completely consume the fermentable sugar present in the brewing wort media. These results demonstrated that combining the FAS2 G1250S mutations with expression of the MaWES mutant enzyme (MaWES_{A360I,A144F}) was successful in increasing ethyl-hexanoate production but additional development was necessary to reduce concomitant production of hexanoic acid and to alleviate strain growth defects.

### Altering Expression of MaWES and FAS2 G1250S Genes to Improve Growth and Ethyl-Hexanoate Production

It was hypothesized that the growth defects observed for strain BY580 may be due to a reduction in essential C16 and C18 fatty acids resulting from the FAS2 G1250S mutation, which, in concert with the anaerobic and high ethanol brewing environment, may inhibit yeast growth. Alternatively or in addition, it was hypothesized that the increased C6-C10 fatty acids produced by the strains inhibited growth by disrupting transmembrane proton gradients, as has previously been reported (see, e.g., Viegas et al. Appl. Environ. Microbiol. (1989). 55:21-28). Altering the expression levels of FAS2 G1250S and the MaWES mutant enzyme (MaWES_{A360I,A144F}) was evaluated to determine effects on the levels of ethyl-hexanol and hexanoic acid produced during fermentation, while also potentially alleviating thee metabolic defects.

Over 30 strains were constructed, each bearing a different combination of yeast-derived promoters driving expression of the MaWES mutant enzyme (MaWES_{A360I,A144F}) and FAS2-G1250S. The native FAS2 locus was unmodified in these strains, such that each strain expressed wild-type FAS2 under the control of the wild-type, native FAS2 promoter, the MaWES mutant enzyme (MaWES_{A360I,A144F}) under the control of a first yeast-derived promoter, and FAS2-G1250S under the control of a second yeast-derived promoter. Each of these strains was grown in small-scale brewing wort fermentations, after which ethyl-hexanoate and hexanoic acid levels were determined. It was found that the promoters driving expression of the MaWES mutant enzyme (MaWES_{A360I,A144F}) and FAS2-G1250S genes had a marked effect on the concentration of ethyl-hexanoate and hexanoic produced, strain growth, and sugar consumption by the strain. One strain, BY845, was found to grow identically to the non-engineered, wild-type control strain, while producing over 3-fold more ethyl-hexanoate and 9-fold as much hexanoic acid as the control strain. Compared to strain BY580, BY845 had improved growth, produced slightly less ethyl-hexanoate, and much less hexanoic acid.

BY845 was used in 5-gallon beer fermentations to assess the growth and ethyl-hexanoate/hexanoic acid production of the strain in a scaled-up brewing environment. Throughout the ten-day fermentation, the sugar consumption profile of BY845 was identical to the control strain. Beer produced by BY845 was characterized as having strong, distinctive pineapple tasting notes, and slight off-flavor notes described as “goaty.” GC/MS analysis of the beer revealed that ethyl-hexanoate and hexanoic acid concentrations were 5.7-fold and 6.8-fold higher in this beer than in the control strain. Specific combinations of promoter sequences driving the expression of the MaWES mutant enzyme (MaWES_{A360I,A144F}) and FAS2 G1250S genes were sufficient to alter the levels and ratios of ethyl-hexanoate and hexanoic acid produced during fermentation and alleviate the growth defects observed in BY719 and BY580. In addition, while the concentration of ethyl-hexanoate produced by BY845 was only 5.7-fold higher than the control strain, this was sufficient to impart strong pineapple flavors in beer. Finally, the hexanoic acid concentrations produced by BY845 were similar to those produced by previous strains, and beer produced by BY845 was perceived as having a goaty off-flavor during beer sensory analysis. These results indicated that yet further development was necessary to decrease hexanoic acid production.

### Expression of Hexanoyl-CoA Synthetase and Deletion of Endogenous AATs to Reduce Hexanoic Acid Production

Two complementary approaches were evaluated to reduce the amount of hexanoic acid produced during fermentation: expression of a hexanoyl-CoA synthetase and deletion of endogenous yeast AAT enzymes.

As described herein, hexanoyl-CoA synthetase (HCS) enzymes catalyze the formation of hexanoyl-CoA from the substrates hexanoic acid and free CoA. Given that this reaction eliminates hexanoic acid while producing hexanoyl-CoA, a precursor for ethyl-hexanoate biosynthesis, expression of an HCS may reduce the levels of hexanoic acid produced by strains like BY845. To test this, strains expressing the MaWES mutant enzyme (MaWES_{A360I,A144F}) and FAS2-G1250S were further engineered to express an HCS enzyme from Cannabis sativa (HCS23) driven by the methylsterol monooxygenase promoter (pERG25), which is considered a moderate strength promoter. These strains were assessed by small-scale wort fermentations followed by GC/MS analysis, which revealed that HCS expression reduced the levels of hexanoic acid in the fermentation media but also led to strain growth defects and incomplete fermentations.

Additional strains were engineered to expression HCS under the control of multiple different yeast-derived promoters to identify an HCS expression regime that did not impede cell growth. Results of these experiments indicated that strain BY888, expressing MaWES, FAS2-G1250S, and HCS with a pHEM13 promoter, which induces strong expression during late stages of fermentation, grew comparably to non-engineered controls strains and produced less hexanoic acid than BY845.

A second approach was explored to reduce hexanoic acid production in strains expressing FAS2-G1250S and the MaWES mutant enzyme (MaWES_{A360I,A144F}), namely deletion of endogenous yeast AAT enzymes, which are thought to produce hexanoic acid through the hydrolysis of ethyl-hexanoate and hexanoyl-CoA. The yeast genome is predicted to encode at least seven AAT enzymes and are thought to have redundant ester and acyl-CoA hydrolysis activities. It was found that single deletion of the endogenous AAT enzyme EEB1 resulted in a modest but significant reduction in hexanoic acid levels in strains expressing FAS2 G1250S and the MaWES mutant enzyme (MaWES_{A360I,A144F}). Interestingly, deletions of several other AATs resulted in growth defects related to sugar consumption during fermentation.

### Example 2

### Generation of Genetically Modified Strains Capable of Producing Increased Levels of Ethyl Hexanoate and Decreased Levels of Hexanoic Acid

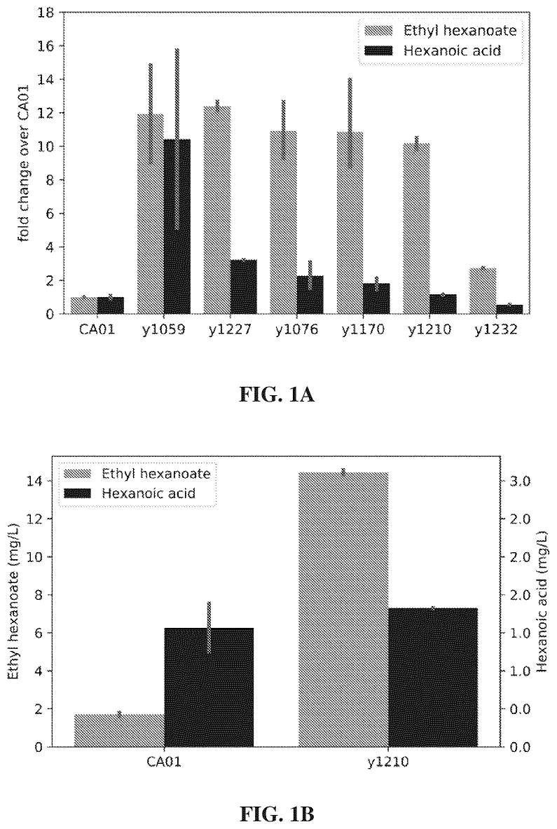

To generate genetically modified strains for beer brewing that produce increased levels of ethyl hexanoate and decreased levels of hexanoic acid, wild-type Saccharomyces cerevisiae strain WLP001 (CA01) were transformed with the constructs shown in Table 1. Transformed strains were grown semi-anaerobically in malt extract fermentations for five days after which ethyl hexanoate and hexanoic acid concentrations were then measured by GC-MS (FIGS. 1A and 1B).

As shown in FIG. 1A, overexpression of FAS2-G1250S and MpAAT1 AA169GF resulted in to an 11.9-fold increase in ethyl hexanoate production and a 10.4-fold increase in production of the off-flavor molecule, hexanoic acid (strain y1059 compared to CA01). Deletion of the endogenous AAT, EHT1, in strain y1059 reduced hexanoic acid production by more than half, while maintaining high levels of ethyl hexanoate production (strain y1227 compared to strain y1059). Deletion of a second endogenous AAT, EEB1, in strain y1227 further reduced hexanoic acid production and modestly decreased ethyl hexanoate production as compared to a strain in which one endogenous AAT was deleted (strain y1076 compared to strain y1227). In addition, deletion of a third endogenous AAT, MGL2, in strain y1170 resulted in modestly reduced hexanoic acid production but did not affect ethyl hexanoate production as compared to a strain in which two endogenous AATs were deleted (strain y1170 compared to strain y1076).

Expression of a hexanoyl-CoA-synthetase (HCS) in strain y1170 further reduced hexanoic acid production without significantly affecting ethyl hexanoate production, as compared to a corresponding strain that did not express the HCS (compare strain y1210 to strain y1170). Strain y1210 was found to produce 14.44 mg/L ethyl hexanoate, a 8.49-fold increase as compared to the level of ethyl hexanoate produced by wild-type CA01, and 1.5 mg/L hexanoic acid, a 1.15-fold increase as compared to the level of hexanoic acid produced by wild-type CA01 (FIG. 1B), and over-expression of a wild-type FAS2 gene and MpAAT1_AA169GF in a strain lacking the endogenous AATs EEB1 and EHT1 results in a 2.7-fold increase in ethyl hexanoate production and a nearly 2-fold reduction in hexanoic acid production.

To generate genetically modified yeast strains for producing wine having increased levels of ethyl hexanoate and decreased levels of hexanoic acid, S. cerevisiae strains EC1118 and Elegance were transformed with the constructs shown in Table 1.

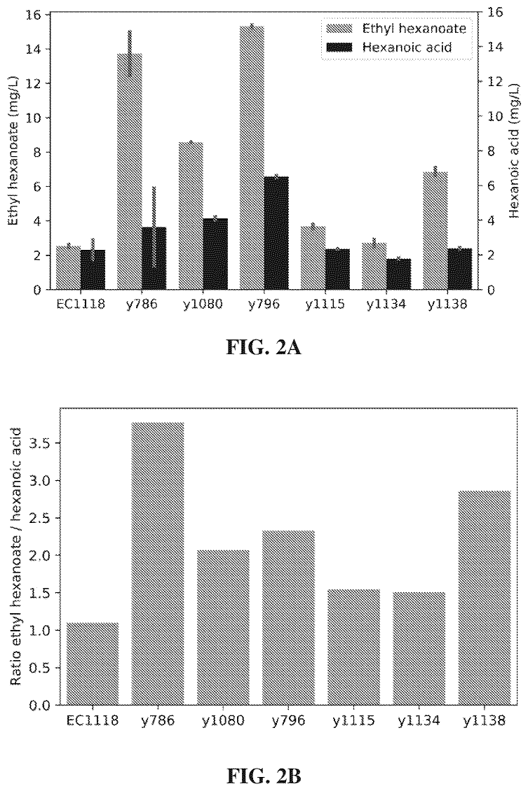

Strains were grown for 14 days in grape juice media, after which ethyl hexanoate and hexanoic acid concentrations in the fermentation media were determined by GC-MS (FIGS. 2A and 2B).

As shown in FIGS. 2A and 2B, genetically modified strains that express FAS2-G1250S as well as a heterologous AAT (either MaWES or MpAAT1) were able to produce increased levels of ethyl hexanoate as compared to the wild-type S. cerevisiae strain EC1118 (strains y786, y796, and y1134 compared to wild-type strain EC1118). With the exception of y1134, the strains tested also produced increased levels of the off-flavor molecule, hexanoic acid. However, for some strains, deletion of the endogenous AATs, EEB1 and EHT1, were found to improve the production ratio of ethyl hexanoate to hexanoic acid as compared to the ratio in strains that contain the endogenous AATs (strain y1134 compared to strain y1138) (see, FIG. 2B).

TABLE 1
Yeast strains assayed in Example 2

| | | Back- | |
|---|---|---|---|
| | Strain | ground | |
| Strain | Type | Strain | Genotype |
| CA01 | Beer | — | Wild-Type |
| y1232 | Beer | CA01 | EHT1::Δ; EEB1:Δ; |
| | | | FIG. 2::pPRB1-FAS2, |
| | | | pPGK1-MpAAT1_AA169GF |
| y1059 | Beer | CA01 | FIG. 2::pPRB1-FAS2_G1250S, |
| | | | pPGK1-MpAAT1_AA169GF |
| y1227 | Beer | CA01 | EHT1::Δ; FIG. 2::pPRB1- |
| | | | FAS2_G1250S, |
| | | | pPGK1-MpAAT1_AA169GF; |
| y1076 | Beer | CA01 | EHT1::Δ; EEB1::Δ; |
| | | | FIG. 2::pPRB1-FAS2_G1250S, |
| | | | pPGK1-MpAAT1_AA169GF |
| y1170 | Beer | CA01 | EHT1::Δ; EEB1::Δ; MGL2::Δ, |
| | | | FIG. 2::pPRB1-FAS2_G1250S, |
| | | | pPGK1-MpAAT1_AA169GF |
| y1210 | Beer | CA01 | EHT1::Δ; EEB1::Δ; MGL2::Δ, |
| | | | FIG. 2::pPRB1-FAS2_G1250S, |
| | | | pPGK1-MpAAT1_AA169GF; |
| | | | pPDC6::pERG25-HCS |
| EC1118 | Wine | — | Wild-Type |
| y796 | Wine | EC1118 | pPDC6::pPRB1-FAS2_G1250S, |
| | | | pQCR10-MaWES1 |
| y1115 | Wine | EC1118 | EHT1::Δ; EEB1::Δ; |
| | | | pPDC6::pPRB1-FAS2_G1250S, |
| | | | pQCR10-MaWES1 |
| y1134 | Wine | EC1118 | pPDC6::pPRB1-FAS2_G1250S, |
| | | | pPGK1-MpAAT1_AA169GF |
| y1138 | Wine | EC1118 | EHT1::Δ; EEB1::Δ; |
| | | | pPDC6::pPRB1-FAS2_G1250S, |
| | | | pPGK1-MpAAT1_AA169GF |
| y786 | Wine | Elegance | pPDC6::pPRB1-FAS2_G1250S, |
| | | | pHEM13-MaWES1 |
| y1080 | Wine | Elegance | EHT1::Δ; EEB1::Δ; |
| | | | pPDC6::pPRB1-FAS2_G1250S, |
| | | | pHEM13-MaWES1 |

What is claimed is:

1. A genetically modified yeast cell (modified cell) comprising:
(i) a first gene operably linked to a first promoter, wherein the first gene is a heterologous gene encoding an enzyme having alcohol-O-acyltransferase (AAT) activity; and
(ii) a second gene operably linked to a second promoter, wherein the second gene encodes an enzyme having fatty acid synthase (FAS2) activity.

2. The modified cell of claim 1, wherein the enzyme having AAT activity is derived from Marinobacter hydrocarbonoclasticus, Fragraia x ananassa, Saccharomyces cerevisiae, Neurospora sitophila, Actinidia deliciosa, Actinidia chinensis, Marinobacter aquaeolei, Saccharornycopsis fibuligera, Malus x domestica, Solanum pennellii, or Solanum lycopersicum.

3. The modified cell of claim 1 or 2, wherein the enzyme having AAT activity comprises a sequence having at least 90% sequence identity to the amino acid sequence set forth in SEQ ID NO: 2-4 or 12-22.

4. The modified cell of any one of claims 1-3, wherein the enzyme having AAT activity does not comprise the sequence of SEQ ID NO: 1.

5. The modified cell of any one of claims 1-4, wherein the enzyme having AAT activity comprises the sequence of SEQ ID NO: 20.

6. The modified cell of any one of claims 1-5, wherein the enzyme having AAT activity comprises at least one substitution mutation at a position corresponding to position A144 and/or A360 of SEQ ID NO: 1.

7. The modified cell of claim 6, wherein the substitution mutation at the position corresponding to position 144 of SEQ ID NO: 1 is a phenylalanine.

8. The modified cell of claim 6 or 7, wherein the substitution mutation at the position corresponding to position 360 of SEQ ID NO: 1 is an isoleucine.

9. The modified cell of any one of claims 1-8, wherein the enzyme having AAT activity comprises at least one substitution mutation at a position corresponding to position A169 and/or A170 of SEQ ID NO: 19.

10. The modified cell of claim 9, wherein the substitution mutation at the position corresponding to position 169 of SEQ ID NO: 19 is a glycine.

11. The modified cell of claim 9 or 10, wherein the substitution mutation at the position corresponding to position 170 of SEQ ID NO: 19 is a phenylalanine.

12. The modified cell of any one of claim 1-3.1, wherein the first enzyme having AAT activity comprises a substitution mutation at a position corresponding to position G150 of a wild-type MhWES2 amino acid sequence.

13. The modified cell of claim 12, wherein the substitution mutation at the position corresponding to position G150 of a wild-type MhWES2 amino acid sequence is a phenylalanine.

14. The modified cell of any one of claims 1-13, wherein the enzyme having FAS2 activity is derived from Saccharomyces cerevisiae.

15. The modified cell of any one of claims 1-14, wherein the enzyme having FAS2 activity comprises a sequence having at least 90% sequence identity to the sequence of SEQ ID NO: 6.

16. The modified cell of claim 15, wherein the enzyme having FAS2 activity does not comprise the sequence of SEQ ID NO: 5.

17. The modified cell of claim 15 or 16, wherein the enzyme having FAS2 activity comprises a substitution mutation at a position corresponding to position 1250 of SEQ ID NO: 5.

18. The modified cell of claim 17, wherein the substitution mutation at the position corresponding to position 1250 of SEQ ID NO: 5 is a serine.

19. The modified cell of any one of claims 1-18, further comprising a third heterologous gene operably linked to a third promoter, wherein the third heterologous gene encodes an enzyme having hexanoyl-CoA synthetase (HCS) activity.

20. The modified cell of claim 19, wherein the enzyme having HCS activity is derived from Cannabis sativa.

21. The modified cell of claim 19 or 20, wherein the enzyme having HCS activity comprises a sequence having at least 90% sequence identity to the sequence of SEQ ID NO: 7.

22. The modified cell of any one of claims 1-21, wherein the first promoter and/or the second promoter is selected from the group consisting of pHEM13, pSPG1, pPRB1, pQCR10, pPGK1, pOLE1, pERG25, and pHHF2.

23. The modified cell of claim 22, wherein:
i) the first promoter is pHEM13, and the second promoter is pSPG1;
ii) the first promoter is pHEM13, and the second promoter is pPRB1;
iii) the first promoter is pQCR10, and the second promoter is pPRB1; or
iv) the first promoter is pPGK, and the second promoter is pPRB1.

24. The modified cell of any one of claims 19-23, wherein the third promoter is selected from the group consisting of pHEM13, pSPG1, pPRB1, pQCR10, pPGK1, pOLE1, pERG25, and pHHF2.

25. The modified cell of claim 24, wherein:
i) the first promoter is pHEM13, the second promoter is pPRB1, and the third promoter is pHEM13;
ii) the first promoter is pQCR10, the second promoter is pPRB1, and the third promoter is pHEM13; or
iii) the first promoter is pPGK1, the second promoter is pPRB1, and the third promoter is pERG25.

26. The modified cell of any one of claims 1-25, wherein the cell has been genetically modified to reduce expression of one or more endogenous AAT enzymes.

27. The modified cell of claim 26, wherein the modified cell does not express endogenous EEB1, EHT1, and/or MGL2.

28. The modified cell of any one of claims 1-27, wherein the yeast cell is of the genus Saccharomyces.

29. The modified cell of claim 28, wherein the yeast cell is of the species Saccharomyces cerevisiae (S. cerevisiae).

30. The modified cell of claim 29, wherein the yeast cell is S. cerevisiae California Ale Yeast strain WLP001, EC-1118, Elegance, Red Star Cote des Blancs, or Epernay II.

31. The modified cell of claim 28, wherein the yeast cell is of the species Saccharomyces pastorianus (S. pastorianus).

32. The modified cell of any one of claims 1-31, wherein growth rate of the modified cell is not substantially impaired relative to a wild-type yeast cell that does not comprise the first heterologous gene and second exogenous gene.

33. The modified cell of claim 32, wherein within one month of the start of fermentation, the modified cell ferments a comparable amount of fermentable sugar to the amount fermented by wild-type yeast cell that does not comprise the first heterologous gene and second exogenous gene.

34. The modified cell of claim 33, wherein within one month of the start of fermentation, the modified cell reduces the amount of fermentable sugars in a medium by at least 95%.

35. The modified cell of any one of claims 1-34, wherein the cell comprises an endogenous gene encoding an enzyme having FAS2 activity.

36. A method of producing a fermented product comprising,
contacting the modified cell of any one of claims 1-35 with a medium comprising at least one fermentable sugar,
wherein the contacting is performed during at least a first fermentation process, to produce a fermented product.

37. The method of claim 36, wherein at least one fermentable sugar is provided in at least one sugar source.

38. The method of claim 36 or 37, wherein the fermentable sugar is glucose, fructose, sucrose, maltose, and/or maltotriose.

39. The method of any one of claims 36-38, wherein the fermented product comprises an increased level of at least one desired product as compared to a fermented product produced by a counterpart cell that does not express the first, second, and/or third heterologous genes, or a counterpart cell that expresses a wild-type enzyme having AAT activity.

40. The method of claim 39, wherein the desired product is ethyl-hexanoate.

41. The method of any one of claims 36-40, wherein the fermented product comprises a reduced level of at least one undesired product as compared to a fermented product produced by a counterpart cell that does not express the first heterologous gene, second exogenous gene, and/or third heterologous genes, or a counterpart cell that expresses a wild-type enzyme having AAT activity.

42. The method of claim 41, wherein the at least one undesired product is hexanoic acid.

43. The method of any one of claims 36-42, wherein the fermented product is a fermented beverage.

44. The method of claim 43, wherein the fermented beverage is beer, wine, sparkling wine (champagne), wine cooler, wine spritzer, hard seltzer, sake, mead, kombucha, or cider.

45. The method of any one of claims 36-44, wherein the sugar source comprises wort, must, fruit juice, honey, rice starch, or a combination thereof.

46. The method of claim 45, wherein the fruit juice is a juice obtained from at least one fruit selected from the group consisting of grapes, apples, blueberries, blackberries, raspberries, currants, strawberries, cherries, pears, peaches, nectarines, oranges, pineapples, mangoes, and passionfruit.

47. The method of claim 45, wherein the sugar source is wort and the method further comprises producing the medium, wherein producing the medium comprises:
(a) contacting a plurality of grains with water; and
(b) boiling or steeping the water and grains to produce wort.

48. The method of claim 47, further comprising adding at least one hop variety to the wort to produce a hopped wort.

49. The method of any one of claims 36-48, further comprising adding at least one hop variety to the medium.

50. The method of claim 45, wherein the sugar source is must and the method further comprises producing the medium, wherein producing the medium comprises crushing a plurality of fruits to produce the must.

51. The method of claim 50, further comprising removing solid fruit material from the must to produce a fruit juice.

52. The method of any one of claims 36-51, further comprising at least one additional fermentation process.

53. The method of any one of claims 36-52, further comprising carbonating the fermented product.

54. A fermented product produced, obtained, or obtainable by the method of any one of claims 36-53.

55. The fermented product of claim 54, wherein the fermented product comprises at least 200 μg/L ethyl-hexanoate.

56. The fermented product of claim 54 or 55, wherein the fermented product comprises less than 10 mg/L hexanoic acid.

57. A method of producing a composition comprising ethanol, the method comprising
contacting the modified cell of any one of claims 1-35 with a medium comprising at least one fermentable sugar,
wherein such contacting is performed during at least a first fermentation process, to produce the composition comprising ethanol.

58. The method of claim 57, wherein at least one fermentable sugar is provided in at least one sugar source.

59. The method of claim 57 or 58, wherein the fermentable sugar is glucose, fructose, sucrose, maltose, and/or maltotriose.

60. The method of any one of claims 57-59, wherein the composition comprising ethanol comprises an increased level of at least one desired product as compared to a composition comprising ethanol produced by a counterpart cell that does not express the first, second, and/or third heterologous genes, or a counterpart cell that expresses a wild-type enzyme having AAT activity.

61. The method of claim 57-60 wherein the desired product is ethyl-hexanoate.

62. The method of any one of claims 57-61, wherein the composition comprising ethanol comprises a reduced level of at least one undesired product as compared to a composition comprising ethanol produced by a counterpart cell that does not express the first heterologous gene, second exogenous gene, and/or third heterologous genes, or a counterpart cell that expresses a wild-type enzyme having AAT activity.

63. The method of claim 62, wherein the at least one undesired product is hexanoic acid.

64. The method of any one of claims 57-63, wherein the composition comprising ethanol is a fermented beverage.

65. The method of claim 64, wherein the fermented beverage is beer, wine, sparkling wine (champagne), wine cooler, wine spritzer, hard seltzer, sake, mead, kombucha, or cider.

66. The method of any one of claims 57-65, wherein the sugar source comprises wort, must, fruit juice, honey, rice starch, or a combination thereof.

67. The method of claim 66, wherein the fruit juice is a juice obtained from at least one fruit selected from the group consisting of grapes, apples, blueberries, blackberries, raspberries, currants, strawberries, cherries, pears, peaches, nectarines, oranges, pineapples, mangoes, and passionfruit.

68. The method of claim 66, wherein the sugar source is wort and the method further comprises producing the medium, wherein producing the medium comprises:
(a) contacting a plurality of grains with water; and
(b) boiling or steeping the water and grains to produce wort.

69. The method of claim 68, further comprising adding at least one hop variety to the wort to produce a hopped wort.

70. The method of any one of claims 57-69, further comprising adding at least one hop variety to the medium.

71. The method of claim 66, wherein the sugar source is must and the method further comprises producing the medium, wherein producing the medium comprises crushing a plurality of fruits to produce the must.

72. The method of claim 71, further comprising removing solid fruit material from the must to produce a fruit juice.

73. The method of any one of claims 57-72, further comprising at least one additional fermentation process.

74. The method of any one of claims 57-73, further comprising carbonating the composition comprising ethanol.

75. A composition comprising ethanol, the composition being produced, obtained, or obtainable by the method of any one of claims 57-74.

76. The composition of claim 75, wherein the composition comprises at least 200 μg/L ethyl-hexanoate.

77. The composition of claim 75 or 76, wherein the composition comprises less than 10 mg/L hexanoic acid.


SEQUENCE LISTING (23 sequences)

SEQ ID NO: 1 - protein, 455 aa, Marinobacter hydrocarbonoclasticus
MTPLNPTDQLFLWLEKRQQPMHVGGLQLFSFPEGAPDDYVAQLADQLRQKTEVTAPFNQRLSYRLGQPVWVEDEHLDLEHHFRFEALPTPGRIRELLSFVSAEHSHLMDRERPMWEVHLIEGLKDRQFALYTKVHHSLVDGVSAMRMATRMLSENPDEHGMPPIWDLPCLSRDRGESDGHSLWRSVTHLLGLSGRQLGTIPTVAKELLKTINQARKDPAYDSIFHAPRCMLNQKITGSRRFAAQSWCLKRIRAVCEAYGTTVNDVVTAMCAAALRTYLMNQDALPEKPLVAFVPVSLRRDDSSGGNQVGVILASLHTDVQEAGERLLKIHHGMEEAKQRYRHMSPEEIVNYTALTLAPAAFHLLTGLAPKWQTFNVVISNVPGPSRPLYWNGAKLEGMYPVSIDMDRLALNMTLTSYNDQVEFGLIGCRRTLPSLQRMLDYLEQGLAELELNAGL

SEQ ID NO: 2 - protein, 453 aa, Fragraia x ananassa
MGEKIEVSINSKHTIKPSTSSTPLQPYKLTLLDQLTPPAYVPIVFFYPITDHDFNLPQTAADLRQALSETLTLYYPLSGRVKNNLYIDDFEEGVPYLEARVNCDMTDFLRLRKIECLNEFVPIKPFSMEAISDERYPLLGVQVNVFDSGIAIGVSVSHKLIDGGTADCFLKSWGAVFRGCRENIIHPSLSEAALLFPPRDDLPEKYVDQMEALWFAGKKVATRRFVFGVKAISSIQDEAKSESVPKPSRVHAVTGFLWKHLIAASRALTSGTTSTRLSIAAQAVNLRTRMNMETVLDNATGNLFWWAQAILELSHTTPEISDLKLCDLVNLLNGSVKQCNGDYFETFKGKEGYGRMCEYLDFQRTMSSMEPAPDIYLFSSWTNFFNPLDFGWGRTSWIGVAGKIESASCKFIILVPTQCGSGIEAWVNLEEEKMAMLEQDPHFLALASPKTLI

SEQ ID NO: 3 - protein, 442 aa, Solanum pennellii
MANTLPISINYHKPKLVVPSSVTPHETKRLSEIDDQGFIRFQIPILMFYKYNSSMKGKDPARIIEDGLSKTLVFYHPLAGRLIEGPNKKLMVNCNGEGVLFIEGDANIELEKLGESIKPPCPYLDLLLHNVPGSDGIIGSPLLLIQVTRFTCGGFAVGFRVSHTMMDGYGFKMFLNALSELIQGASTPSILPVWQRHLLSARSSPCITCSHHEFDEEIESKIAWESMEDKLIQESFFFGNEEMEVIKNQIPPNYGCTKFELLMAFLWKCRTIALDLHPEEIVRLTYVINIRGKKSLNIELPIGYYGNAFVTPVVVSKAGLLCSNPVTYAVELIKKVKDHINEEYIKSVIDLTVIKGRPELTKSWNFLVSDNRYIGFDEFDFGWGNPIFGGISKATSFISFGVSVKNDKGEKGVLIAISLPPLAMKKLQDIYNMTFRVIIPRI

SEQ ID NO: 4 - protein, 455 aa, Artificial Sequence
  Synthetic
MTPLNPTDQLFLWLEKRQQPMHVGGLQLFSFPEGAPDDYVAQLADQLRQKTEVTAPFNQRLSYRLGQPVWVEDEHLDLEHHFRFEALPTPGRIRELLSFVSAEHSHLMDRERPMWEVHLIEGLKDRQFALYTKVHHSLVDGVSFMRMATRMLSENPDEHGMPPIWDLPCLSRDRGESDGHSLWRSVTHLLGLSGRQLGTIPTVAKELLKTINQARKDPAYDSIFHAPRCMLNQKITGSRRFAAQSWCLKRIRAVCEAYGTTVNDVVTAMCAAALRTYLMNQDALPEKPLVAFVPVSLRRDDSSGGNQVGVILASLHTDVQEAGERLLKIHHGMEEAKQRYRHMSPEEIVNYTALTLAPAIFHLLTGLAPKWQTFNVVISNVPGPSRPLYWNGAKLEGMYPVSIDMDRLALNMTLTSYNDQVEFGLIGCRRTLPSLQRMLDYLEQGLAELELNAGL

SEQ ID NO: 5 - protein, 1889 aa, Saccharomyces cerevisiae
MKPEVEQELAHILLTELLAYQFASPVRWIETQDVFLKDFNTERVVEIGPSPTLAGMAQRTLKNKYESYDAALSLHREILCYSKDAKEIYYTPDPSELAAKEEPAKEEAPAPTPAASAPAPAAAAPAPVAAAAPAAAAAEIADEPVKASLLLHVLVAHKLKKSLDSIPMSKTIKDLVGGKSTVQNEILGDLGKEFGTTPEKSEETPLEELAETFQDTFSGALGKQSSSLLSRLISSKMPGGFTITVARKYLQTRWGLPSGRQDGVLLVALSNEPAARLGSEADAKAFLGSMAQKYASIVGVDLSSAASASGAAGAGAAAGAAMIDAGALEEITKDHKVLARQQLQVLARYLKMDLDNGERKFLKEKDTVAELQAQLDYLNAELGEFFVNGVATSFSRKKARTFDSSWNWAKQSLLSLYFEIIHGVLKNVDREVVSEAINIMNRSNDALIKFMEYHISNTDETKGENYQLVKTLGEQLIENCKQVLDVDPVYKDVAKPTGPKTAIDKNGNITYSEEPREKVRKLSQYVQEMALGGPITKESQPTIEEDLTRVYKAISAQADKQDISNSTRVEFEKLYSDLMKFLESSKEIDPSQTTQLAGMDVEDALDKDSTKEVASLPNKSTISKTVSSTIPRETIPFLHLRKKTPAGDWKYDRQLSSLFLDGLEKAAFNGVTFKDKYVLITGAGKGSIGAEVLQGLLQGGAKVVVTTSRFSKQVTDYYQSIYAKYGAKGSTLIVVPFNQGSKQDVEALIEFIYDTEKNGGLGWDLDAIIPFAAIPEQGIELEHIDSKSEFAHRIMLTNILRMMGCVKKQKSARGIETRPAQVILPMSPNHGTFGGDGMYSESKLSLETLFNRWHSESWANQLTVCGAIIGWTRGTGLMSANNIIAEGIEKMGVRTFSQKEMAFNLLGLLTPEVVELCQKSPVMADLNGGLQFVPELKEFTAKLRKELVETSEVRKAVSIETALEHKVVNGNSADAAYAQVEIQPRANIQLDFPELKPYKQVKQIAPAELEGLLDLERVIVVTGFAEVGPWGSARTRWEMEAFGEFSLEGCVEMAWIMGFISYHNGNLKGRPYTGWVDSKTKEPVDDKDVKAKYETSILEHSGIRLIEPELFNGYNPEKKEMIQEVIVEEDLEPFEASKETAEQFKHQHGDKVDIFEIPETGEYSVKLLKGATLYIPKALRFDRLVAGQIPTGWNAKTYGISDDIISQVDPITLFVLVSVVEAFIASGITDPYEMYKYVHVSEVGNCSGSGMGGVSALRGMFKDRFKDEPVQNDILQESFINTMSAWVNMLLISSSGPIKTPVGACATSVESVDIGVETILSGKARICIVGGYDDFQEEGSFEFGNMKATSNTLEEFEHGRTPAEMSRPATTTRNGFMEAQGAGIQIIMQADLALKMGVPIYGIVAMAATATDKIGRSVPAPGKGILTTAREHHSSVKYASPNLNMKYRKRQLVTREAQIKDWVENELEALKLEAEEIPSEDQNEFLLERTREIHNEAESQLRAAQQQWGNDFYKRDPRIAPLRGALATYGLTIDDLGVASFHGTSTKANDKNESATINEMMKHLGRSEGNPVIGVFQKFLTGHPKGAAGAWMMNGALQILNSGIIPGNRNADNVDKILEQFEYVLYPSKTLKTDGVRAVSITSFGFGQKGGQAIVVHPDYLYGAITEDRYNEYVAKVSAREKSAYKFFHNGMIYNKLFVSKEHAPYTDELEEDVYLDPLARVSKDKKSGSLTFNSKNIQSKDSYINANTIETAKMIENMTKEKVSNGGVGVDVELITSINVENDTFIERNFTPQEIEYCSAQPSVQSSFAGTWSAKEAVFKSLGVKSLGGGAALKDIEIVRVNKNAPAVELHGNAKKAAEEAGVTDVKVSISHDDLQAVAVAVSTKKGS

SEQ ID NO: 6 - protein, 1889 aa, Artificial Sequence
  Synthetic
MKPEVEQELAHILLTELLAYQFASPVRWIETQDVFLKDFNTERVVEIGPSPTLAGMAQRTLKNKYESYDAALSLHREILCYSKDAKEIYYTPDPSELAAKEEPAKEEAPAPTPAASAPAPAAAAPAPVAAAAPAAAAAEIADEPVKASLLLHVLVAHKLKKSLDSIPMSKTIKDLVGGKSTVQNEILGDLGKEFGTTPEKSEETPLEELAETFQDTFSGALGKQSSSLLSRLISSKMPGGFTITVARKYLQTRWGLPSGRQDGVLLVALSNEPAARLGSEADAKAFLGSMAQKYASIVGVDLSSAASASGAAGAGAAAGAAMIDAGALEEITKDHKVLARQQLQVLARYLKMDLDNGERKFLKEKDTVAELQAQLDYLNAELGEFFVNGVATSFSRKKARTFDSSWNWAKQSLLSLYFEIIHGVLKNVDREVVSEAINIMNRSNDALIKFMEYHISNTDETKGENYQLVKTLGEQLIENCKQVLDVDPVYKDVAKPTGPKTAIDKNGNITYSEEPREKVRKLSQYVQEMALGGPITKESQPTIEEDLTRVYKAISAQADKQDISNSTRVEFEKLYSDLMKFLESSKEIDPSQTTQLAGMDVEDALDKDSTKEVASLPNKSTISKTVSSTIPRETIPFLHLRKKTPAGDWKYDRQLSSLFLDGLEKAAFNGVTFKDKYVLITGAGKGSIGAEVLQGLLQGGAKVVVTTSRFSKQVTDYYQSIYAKYGAKGSTLIVVPFNQGSKQDVEALIEFIYDTEKNGGLGWDLDAIIPFAAIPEQGIELEHIDSKSEFAHRIMLTNILRMMGCVKKQKSARGIETRPAQVILPMSPNHGTFGGDGMYSESKLSLETLFNRWHSESWANQLTVCGAIIGWTRGTGLMSANNIIAEGIEKMGVRTFSQKEMAFNLLGLLTPEVVELCQKSPVMADLNGGLQFVPELKEFTAKLRKELVETSEVRKAVSIETALEHKVVNGNSADAAYAQVEIQPRANIQLDFPELKPYKQVKQIAPAELEGLLDLERVIVVTGFAEVGPWGSARTRWEMEAFGEFSLEGCVEMAWIMGFISYHNGNLKGRPYTGWVDSKTKEPVDDKDVKAKYETSILEHSGIRLIEPELFNGYNPEKKEMIQEVIVEEDLEPFEASKETAEQFKHQHGDKVDIFEIPETGEYSVKLLKGATLYIPKALRFDRLVAGQIPTGWNAKTYGISDDIISQVDPITLFVLVSVVEAFIASGITDPYEMYKYVHVSEVGNCSGSSMGGVSALRGMFKDRFKDEPVQNDILQESFINTMSAWVNMLLISSSGPIKTPVGACATSVESVDIGVETILSGKARICIVGGYDDFQEEGSFEFGNMKATSNTLEEFEHGRTPAEMSRPATTTRNGFMEAQGAGIQIIMQADLALKMGVPIYGIVAMAATATDKIGRSVPAPGKGILTTAREHHSSVKYASPNLNMKYRKRQLVTREAQIKDWVENELEALKLEAEEIPSEDQNEFLLERTREIHNEAESQLRAAQQQWGNDFYKRDPRIAPLRGALATYGLTIDDLGVASFHGTSTKANDKNESATINEMMKHLGRSEGNPVIGVFQKFLTGHPKGAAGAWMMNGALQILNSGIIPGNRNADNVDKILEQFEYVLYPSKTLKTDGVRAVSITSFGFGQKGGQAIVVHPDYLYGAITEDRYNEYVAKVSAREKSAYKFFHNGMIYNKLFVSKEHAPYTDELEEDVYLDPLARVSKDKKSGSLTFNSKNIQSKDSYINANTIETAKMIENMTKEKVSNGGVGVDVELITSINVENDTFIERNFTPQEIEYCSAQPSVQSSFAGTWSAKEAVFKSLGVKSLGGGAALKDIEIVRVNKNAPAVELHGNAKKAAEEAGVTDVKVSISHDDLQAVAVAVSTKKGS

SEQ ID NO: 7 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 8 - DNA, 801 bp, Saccharomyces cerevisiae
taatgtagaaggttgagaacaaccggatcttgcggtcatttttcttttcgaggaaagtgcaagtctgccactttccagaaggcatagccttgcccttttgttgatatttctccccaccgtaattgttgcattcgcgatcttttcaacaatacattttatcatcaagcccgcaaatcctctggagtttgtcctctcgttcactgttgggaaaaacaatacgcctaattcgtgattaagattcttcaaaccatttcctgcggagtttttactgtgtgttgaacggttcacagcgtaaaaaaaagttactataggcacggtattttaatttcaattgtttagaaagtgccttcacaccattagcccctgggattaccgtcataggcactttctgctgagctcctgcgagatttctgcgctgaaagagtaaaagaaatctttcacagcggctccgcgggcccttctacttttaaacgagtcgcaggaacagaagccaaatttcaaagaacgctacgctttcgccttttctggttctcccaccaataacgctccagcttgaacaaagcataagactgcaaccaaagcgctgacggacgatccgaagataaagcttgctttgcccattgttctcgtttcgaaaggctatataaggacacggattttccttttttttttccacctattgtctttctttgttaagcttttattctccgggttttttttttttgagcatatcaaaagctttcttttcgcaaatcaaacatagcaaaccgaactcttcgaacacaattaaatacacataaa

SEQ ID NO: 9 - DNA, 750 bp, Saccharomyces cerevisiae
atgaagttcacttcacatccaatgagaaaaacaaaatccgcagggctatcacccagaacatcctccacttcatcttcttcaggacagagaaaagcgcatcaccaccaccatcaccacaaccacgtttcaaggacgaaaactaccgaaagcaccaaatcaggcaacagcaaaaaggacagttcctcatcctcaacaaacgaccatcaatttaaaaggtctgaaaagaagaaaaaaagtaaatttggctcgatcttcaaaaaagttttcggatgaaccggattaatacaagtaaaatcagcaaagatatagaagacaaaataagcgtgaaaacaatcataaaccactcacaacgggggttttcagctgttactcctccatacatacattttgataaagatataatgttatatttcttttcgtaattttgttttacttcggtttgctctatagatttcatcagccgcaccgaaaagggagatcaataaggtaccctttaaaagggataagaagcctaacatcaccccaataaatggagtaatggccagcattggatgaagagaagaattacgggatactgggataacactgttaaaaatgcttcgcgacgtgagggtcttatataaattgaactgccaaatctctttcacattatccaggatagtttggaatgtgtgttactgaaagatcagaatcaataaatacaatcaatacaaatatttagcgcataaaattcaaacaaagtttactgaa

SEQ ID NO: 10 - DNA, 750 bp, Saccharomyces cerevisiae
cgagaaacagggggggagaaaaggggaaaagagaaggaaagaaagactcatctatcgcagataagacaatcaaccctcatggcgcctccaaccaccatccgcactagggaccaagcgctcgcaccgttagcaacgcttgactcacaaaccaactgccggctgaaagagcttgtgcaatgggagtgccaattcaaaggagccgaatacgtctgttcgccttttaagaggctttttgaacactgcattgcacccgacaaatcagccactaactacgaggtcacggatacatataccaatagttaaaaaattacatatactctatatagcacagtagtgtgataaataaaaaattttgccaagacttttttaaactgcacccgacagatcaggtctgtgcctactatgcacttatgcccggggtcccgggaggagaaaaaacgagggctgggaaatgtccgtggacttaaaacgctccgggttagcagagtagcagggctttcggctttggaaatttaggtgacttgttgaaaaagcaaaatttgggctcagtaatgccacagcagtggcttatcacgccaggactgcgggagtggcgggggcaaacacacccgcgataaagagcgcgatgaatataaaagggggccaatgttacgtcccgttatattggagttcttcccatacaaacttaagagtccaattagcttcatcgccaataaaaaaacaaactaaacctaattctaacaagcaaag

SEQ ID NO: 11 - protein, 1887 aa, Artificial Sequence
  Synthetic
MKPEVEQELAHILLTELLAYQFASPVRWIETQDVFLKDFNTERVVEIGPSPTLAGMAQRTLKNKYESYDAALSLHREILCYSKDAKEIYYTPDPSELAAKEEPAKEEAPAPTPAASAPAPAAAAPAPVAAAAPAAAAAEIADEPVKASLLLHVLVAHKLKKSLDSIPMSKTIKDLVGGKSTVQNEILGDLGKEFGTTPEKPEETPLEELAETFQDTFSGALGKQSSSLLSRLISSKMPGGFTITVARKYLQTRWGLPSGRQDGVLLVALSNEPAARLGSEADAKAFLDSMAQKYASIVGVDLSSAASASGAAGAGAAAGAAMIDAGALEEITKDHKVLARQQLQVLARYLKMDLDNGERKFLKEKDTVAELQAQLDYLNAELGEFFVNGVATSFSRKKARTFDSSWNWAKQSLLSLYFEIIHGVLKNVDREVVSEAINIMNRSNDALIKFMEYHISNTDETKGENYQLVKTLGEQLIENCKQVLDVDPVYKDVAKPTGPKTAIDKNGNITYSEEPREKVRKLSQYVQEMALGGPITKESQPTIEEDLTRVYKAISAQADKQDISSSTRVEFEKLYSDLMKFLESSKEIDPSQTTQLAGMDVEDALDKDSTKEVASLPNKSTISKTVSSTIPRETIPFLHLRKKTPAGDWKYDRQLSSLFLDGLEKAAFNGVTFKDKYVLITGAGKGSIGAEVLQGLLQGGAKVVVTTSRFSKQVTDYYQSIYAKYGAKGSTLIVVPFNQGSKQDVEALIEFIYDTEKNGGLGWDLDAIIPFAAIPEQGIELEHIDSKSEFAHRIMLTNILRMMGCVKKQKSARGIETRPAQVILPMSPNHGTFGGDGMYSESKLSLETLFNRWHSESWANQLTVCGAIIGWTRGTGLMSANNIIAEGIEKMGVRTFSQKEMAFNLLGLLTPEVVELCQKSPVMADLNGGLQFVPELKEFTAKLRKELVETSEVRKAVSIETALEHKVVNGNSADAAYAQVEIQPRANIQLDFPELKPYKQVKQIAPAELEGLLDLERVIVVTGFAEVGPWGSARTRWEMEAFGEFSLEGCVEMAWIMGFISYHNGNLKGRPYTGWVDSKTKEPVDDKDVKAKYETSILEHSGIRLIEPELFNGYNPEKKEMIQEVIVEEDLEPFEASKETAEQFKHQHGDKVDIFEIPETGEYSVKLLKGATLYIPKALRFDRLVAGQIPTGWNAKTYGISDDIISQVDPITLFVLVSVVEAFIASGITDPYEMYKYVHVSEVGNCSGSGMGGVSALRGMFKDRFKDEPVQNDILQESFINTMSAWVNMLLISSSGPIKTPVGACATSVESVDIGVETILSGKARICIVGGYDDFQEEGSFEFGNMKATSNTLEEFEHGRTPAEMSRPATTTRNGFMEAQGAGIQIIMQADLALKMGVPIYGIVAMAATATDKIGRSVPAPGKGILTTAREHHSSVKYASPNLNMKYRKRQLVTREAQIKDWVENELEALKLEAEEIPSEDQNEFLLERTREIHNEAESQLRAAQQQWGNDFYKRDPRIAPLRGALATYGLTIDDLGVASFHGTSTKANDKNESATINEMMKHLGRSEGNPVIGVFQKFLTGHPKGAAGAWMMNGALQILNSGIIPGNRNADNVDKILEQFEYVLYPSKTLKTDGVRAVSITSFGFGQKGGQAIVVHPDYLYGAITEDRYNEYVAKVSAREKSAYKFFHNGMIYNKLFVSKEHAPYTDELEEDVYLDPLARVSKDKKSGSLTFNSKNIQSKDSYINANTIETAKMIENMTKEKVSNGGVGVDVELITSINVENDTFIERNFTPQEIEYCSAQPSVQSSFAGTWSAKEAVFKSLGVKSLGGGAALKDIEIVRVNKNAPAVELHGNAKKAAEEAGVTDVKVSISHDDLQAVAVAVSTKK

SEQ ID NO: 12 - protein, 525 aa, Saccharomyces cerevisiae
MNEIDEKNQAPVQQECLKEMIQNGHARRMGSVEDLYVALNRQNLYRNFCTYGELSDYCTRDQLTLALREICLKNPTLLHIVLPTRWPNHENYYRSSEYYSRPHPVHDYISVLQELKLSGVVLNEQPEYSAVMKQILEEFKNSKGSYTAKIFKLTTTLTIPYFGPTGPSWRLICLPEEHTEKWKKFIFVSNHCMSDGRSSIHFFHDLRDELNNIKTPPKKLDYIFKYEEDYQLLRKLPEPIEKVIDFRPPYLFIPKSLLSGFIYNHLRFSSKGVCMRMDDVEKTDDVVTEIINISPTEFQAIKANIKSNIQGKCTITPFLHVCWFVSLHKWGKFFKPLNFEWLTDIFIPADCRSQLPDDDEMRQMYRYGANVGFIDFTPWISEFDMNDNKENFWPLIEHYHEVISEALRNKKHLHGLGFNIQGFVQKYVNIDKVMCDRAIGKRRGGTLLSNVGLFNQLEEPDAKYSICDLAFGQFQGSWHQAFSLGVCSTNVKGMNIVVASTKNVVGSQESLEELCSIYKALLLGP

SEQ ID NO: 13 - protein, 501 aa, Neurospora sitophila
MGTSIPQPIRPLGPCEAYSSSRHALGFYRCLANTCRYAVPWSVLQGKSVPDVLEAAIANLVLRLPRLSVAITGDEASRPYFASVSSLDLSYHLECVELRAELDFHARDSHLLHMLEAQHNQLWPDVGFRPPWKVLAVYDPRPSQLEDRLILDIVLAIHHSLADGRSTAIFQTSLLDELNKPPVRPSCLEDHVLRMPSKPHGHILPPQEELVKFTTSWRFLAGTLWNEFVSGWLYKPATDLPWAGAPIRPDPYQTRLRLVTIPAKAVSQLLTNCRANETTLTPLLHVLILTSLARRLTAEAATSFQSCTPVDLRPFIQSGSHVADPAEVFGVLVTSASHSFNSSRVSGLREQASGEKIWSLAQTLRQELKDRLEAIPQDDMVSMLRWIANWRGFWLNKVNKPREHTLEVSNIGSLHGSPEKTANADLETGSKWQIVRSVMSQCAIVAGPALCASVSGVVGGPISIALSWQEGIIESELVEGVAHDLQLWMNQGGPVHGQRLP

SEQ ID NO: 14 - protein, 453 aa, Fragaria x ananassa
MGEKIEVSINSKHTIKPSTSSTPLQPYKLTLLDQLTPPAYVPIVFFYPITDHDFNLPQTLADLRQALSETLTLYYPLSGRVKNNLYIDDFEEGVPYLEARVNCDMTDFLRLRKIECLNEFVPIKPFSMEAISDERYPLLGVQVNVFDSGIAIGVSVSHKLIDGGTADCFLKSWGAVFRGCRENIIHPSLSEAALLFPPRDDLPEKYVDQMEALWFAGKKVATRRFVFGVKAISSIQDEAKSESVPKPSRVHAVTGFLWKHLIAASRALTSGTTSTRLSIAAQAVNLRTRMNMETVLDNATGNLFWWAQAILELSHTTPEISDLKLCDLVNLLNGSVKQCNGDYFETFKGKEGYGRMCEYLDFQRTMSSMEPAPDIYLFSSWTNFFNPLDFGWGRTSWIGVAGKIESASCKFIILVPTQCGSGIEAWVNLEEEKMAMLEQDPHFLALASPKTLI

SEQ ID NO: 15 - protein, 432 aa, Actinidia deliciosa
MASSVRLVKKPVLVAPVDPTPSTVLSLSSLDSQLFLRFPIEYLLVYASPHGVDRAVTAARVKAALARSLVPYYPLAGRVKTRPDSTGLDVVCQAQGAGLLEAVSDYTASDFQRAPRSVTEWRKLLLVEVFKVVPPLVVQLTWLSDGCVALGVGFSHCVIDGIGSSEFLNLFAELATGRARLSEFQPKPVWDRHLLNSAGRTNLGTHPEFGRVPDLSGFVTRFTQERLSPTSITFDKTWLKELKNIAMSTSQPGEFPYTSFEVLSGHIWRSWARSLNLPAKQVLKLLFSINIRNRVKPSLPAGYYGNAFVLGCAQTSVKDLTEKGLGYCADLVRGAKERVGDEYAREVVESVSWPRRASPDSVGVLIISQWSRLGLDRVDFGLGRPVQVGPICCDRYCLFLPVRDRTESVKVMVAVPTSAVDRYEYFIRSPYS

SEQ ID NO: 16 - protein, 456 aa, Actinidia chinensis
MASFPPSLVFTVRRNEPTLVLPSKSTPRELKQLSDIDDQEGLRFQVPVIMFYKRKLSMEGEDPVKVIREALAEALVFYYPFAGRLIEGPNRKLMVDCTGEGVLFIEADADIEVNQLIGDTIDPGFSYLDELLHDVPGSEGILGCPLLLIQVTRFRCGGWAFAIRLNHTMSDAPGLVQLLTTIAEFARGAEGAPSVPPVWQREFLAARQPPSITFQHHEYEQVINTTTDDNKSMTHKSFFFGPKEIRAIRSHFPPHYRSVSSTFDVLTACLWRCRTCALGLDPPKTVRISCAANGRGKHDLHVPRGYYGNVFAFPAVVSRAGMISTSSLEYTVEEVKKAKARMTGEYLRSVADLMVTKGRPLYTVAGNYIVSDTTRVGFDAIDFGWGKPVYGGPARAFPLISFYARFKNNRGEDGTVVLICLPEAAMKRFQDELKKMTEEHVDGPFEYKLIKVMSKL

SEQ ID NO: 17 - protein, 554 aa, Saccharomycopsis fibuligera
MTSETLQTSSSSFPASEASQKDSTPAQTTQTAQKQGPVKSKDDLTYKAPFLERNFYFSSKHELFNCFGVSIVVNKPISREQFYVALRKIILKYPKSITSVYDEFDREHHLRFIPKTKIIFDDNAVEFNEKFDQYPYQNKELSALLTSYRFDADPNNGKPSWKIVYFPKIKMLSWLFDHPISDGASGAAFCKELVESLNYITQKELDEAKDLFESSAANKKAVELFNLEKDISKFENPITPDSFKIAGYKPSLAEKIGTPILRFFLDKFPKLFPLVIEGEMHKQQFVDTKPIKFDNKKFFVREQDVISKDSPLCGQALTYIRIDPETTAKILQQCRNNNTKFQTTFMMVFLSTIHEIAPEAYTNKYLKIVTAANFRHIFPNYKYGHSKFLSKPDSYTKETGQFKDGFHDHAVVFYVEPFKKFNWNLVQKYHNFLHKLIRSKQWFSGYYLASEAVSAKTFFDQKIGTHDDTYFALTNLGFVDLIDHGEEASNKYQIEDLIFTASPGPMTGTHSAVLTSTKNGINICVADQDPAINSEEFRARLTENLRKLAESGNV

SEQ ID NO: 18 - protein, 522 aa, Saccharomycopsis fibuligera
MGNFQFSRNDFYTDPTFTEKCFYYYDQYGLISNFSVTIKTTASITRELLYAALKKVILKYPNLVSSIHDKFDYDTHNEKTLTKSPKKIIYFDDNIVQFISQDEETRNYADINQIQLLLNATKFDSNFTNGKPMWKIFVFPNKNLTSWVFDYSIFDGGSAIVYQKELVEALNQILESEQQKAREILDNASKRTTPILFDFEKDWPLFQRAPSQGIFKEINYVPSIFKKVSSQVIKLLSNAVPDKTIDELNDEANKSAFLERIIFEKEKLYLSKNVIGLESGAAKPLSKIININHIILSKILDKCHTKGCNFQAIFIIIFLATVHQVIPLQYSKKYLKTVTSASFRNIFTKQFVSHNEYLAEQELGIQKLLQGQQQFIDGIFVHSAIIYIEPFDEFSWELCHKYDSFLHTLLHSKGWFANYYVANRGIQAKAFVDNKLGSQDDVFVSFDNLGLVRVKESGKFQIEDIIFTKAPDPIAGDNLIAMVSTKKGGLNIQINIAEEHIQARFDEFCLRLSENLIALGNF

SEQ ID NO: 19 - protein, 455 aa, Malus x domestica
MMSFSVLQVKRLQPELITPAKSTPQETKFLSDIDDQESLRVQIPIIMCYKDNPSLNKNRNPVKAIREALSRALVYYYPLAGRLREGPNRKLVVDCNGEGILFVEASADVTLEQLGDKILPPCPLLEEFLYNFPGSDGIIDCPLLLIQVTCLTCGGFILALRLNHTMCDAAGLLLFLTAIAEMARGAHAPSILPVWERELLFARDPPRITCAHHEYEDVIGHSDGSYASSNQSNMVQRSFYFGAKEMRVLRKQIPPHLISTCSTFDLITACLWKCRTLALNINPKEAVRVSCIVNARGKHNNVRLPLGYYGNAFAFPAAISKAEPLCKNPLGYALELVKKAKATMNEEYLRSVADLLVLRGRPQYSSTGSYLIVSDNTRVGFGDVNFGWGQPVFAGPVKALDLISFYVQHKNNTEDGILVPMCLPSSAMERFQQELERITQEPKEDICNNLRSTSQ

SEQ ID NO: 20 - protein, 455 aa, Artificial Sequence
  Synthetic
MMSFSVLQVKRLQPELITPAKSTPQETKFLSDIDDQESLRVQIPIIMCYKDNPSLNKNRNPVKAIREALSRALVYYYPLAGRLREGPNRKLVVDCNGEGILFVEASADVTLEQLGDKILPPCPLLEEFLYNFPGSDGIIDCPLLLIQVTCLTCGGFILALRLNHTMCDGFGLLLFLTAIAEMARGAHAPSILPVWERELLFARDPPRITCAHHEYEDVIGHSDGSYASSNQSNMVQRSFYFGAKEMRVLRKQIPPHLISTCSTFDLITACLWKCRTLALNINPKEAVRVSCIVNARGKHNNVRLPLGYYGNAFAFPAAISKAEPLCKNPLGYALELVKKAKATMNEEYLRSVADLLVLRGRPQYSSTGSYLIVSDNTRVGFGDVNFGWGQPVFAGPVKALDLISFYVQHKNNTEDGILVPMCLPSSAMERFQQELERITQEPKEDICNNLRSTSQ

SEQ ID NO: 21 - protein, 474 aa, Artificial Sequence
  Synthetic
MGKRLGTLDASWLAVESEDTPMHVGTLQIFSLPEGAPETFLRDMVTRMKEAGDVAPPWGYKLAWSGFLGRVIAPAWKVDKDIDLDYHVRHSALPRPGGERELGILVSRLHSNPLDFSRPLWECHVIEGLENNRFALYTKMHHSMIDGISFVRLMQRVLTTDPERCNMPPPWTVRPHQRRGAKTDKEASVPAAVSQAMDALKLQADMAPRLWQAGNRLVHSVRHPEDGLTAPFTGPVSVLNHRVTAQRRFATQHYQLDRLKNLAHASGGSLNDIVLYLCGTALRRFLAEQNNLPDTPLTAGIPVNIRPADDEGTGTQISFMIASLATDEADPLNRLQQIKTSTRRAKEHLQKLPKSALTQYTMLLMSPYILQLMSGLGGRMRPVFNVTISNVPGPEGTLYYEGARLEAMYPVSLIAHGGALNITCLSYAGSLNFGFTGCRDTLPSMQKLAVYTGEALDELESLILPPKKRARTRK

SEQ ID NO: 22 - DNA, 696 bp, Saccharomyces cerevisiae
gagagctggccaaaaagagggccgaagacggcgttgaatttcattcaaaactatttagaagggcagagccaggtgaggatttagattattatatttacaagcacatccctgaagggaccgacaagcatgaagaacagatcaggagcattttggaaactgccccgattttaccaggacaggcattcactgaaaaattttctattccggcttataaaaagcatggaatccaaaagaattaggcttctcattctattttaattatactagtacgatttctcactctgtaatttaatatcagtgtaatatgcacctagttatgggtagtttttgctaacgttacgagccgcgaaactgtcctcaatcttcaccactacctctaatgactgaagaatgctatgcgatataacgctgccgcactttgaatatatacttatatttacatagttttcaagtgcgtattactattgcaaagtagtattttgtcacgtgattttgatccaattaaaactaaatatggttcaacccgttgtttccgcatcaaaaaaccataccatttatcaaggggacgggatatatcacataacagtttgaatgcataatttgttatagatatcttctggaataatcttcacagcaaaagcgcaagtcgaataatatatcgataaatacaatccataagacttaaaactaacctca

SEQ ID NO: 23 - protein, 5 aa, Artificial Sequence
  Synthetic
  MISC_FEATURE (1)..(1): Xaa may be Asn or Asp
XFGWG